(19) United States
(12) Patent Application Publication
(10) Pub. No.: US 2011/0237566 A1
(43) Pub. Date: Sep. 29, 2011

(54) Therapeutic Macrolide Compounds and Their Use

(75) Inventors: Rustum S. Boyce, Scarsdale, NY (US); Muhammad Sofian Asi Sihombing, Singapore (SG); Hiroki Sone, Singapore (SG)

(73) Assignee: MERLION PHARMACEUTICALS PTE LTD, Singapore (SG)

(21) Appl. No.: 13/130,144

(22) PCT Filed: Nov. 24, 2009
(86) PCT No.: PCT/SG09/00446

§ 371 Date: May 19, 2011

Related U.S. Application Data
(60) Provisional application No. 61/117,591, filed on Nov. 25, 2008.

(30) Foreign Application Priority Data
Nov. 25, 2008 (GB) 0821539.4

(51) Int. Cl. A61K 31/553; C07D 313/00; C07D 413/14; C07D 405/14; C07D 267/10; C07D 243/08; A61K 31/336; A61K 31/5355; A61K 31/497; A61K 31/4523; A61K 31/551; A61P 35/00

(52) U.S. Cl. 514/211.01; 549/346; 544/147; 544/374; 546/207; 540/544; 540/575; 514/450; 514/231.5; 514/254.1; 514/326; 514/218

(57) ABSTRACT

The present invention pertains generally to the field of therapeutic compounds, and more specifically to certain macrolide compounds (for convenience, collectively referred to herein as “MC compounds”), which, inter alia, are useful in treatment of cancer. The present invention also pertains to pharmaceutical compositions comprising such compounds, and the use of such compounds and compositions, both in vitro and in vivo, to treat proliferative conditions such as cancer, and in the treatment of diseases and conditions that are mediated by the regulation (e.g. inhibition) of cell proliferation, optionally in combination with another agent.

### TECHNICAL FIELD

The present invention pertains generally to the field of therapeutic compounds, and more specifically to certain macrolide compounds (for convenience, collectively referred to herein as “MC compounds”), which, inter alia, are useful in the treatment of proliferative conditions such as cancer. The present invention also pertains to pharmaceutical compositions comprising such compounds, and the use of such compounds and compositions, both in vitro and in vivo, to treatment of proliferative conditions such as cancer, etc., optionally in combination with another agent.

### BACKGROUND

A number of patents and publications are cited herein in order to more fully describe and disclose the invention and the state of the art to which the invention pertains. Each of these references is incorporated herein by reference in its entirety into the present disclosure, to the same extent as if each individual reference was specifically and individually indicated to be incorporated by reference.

Throughout this specification, including the claims which follow, unless the context requires otherwise, the word “comprise,” and variations such as “comprises” and “comprising,” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Thus, for example, reference to “a pharmaceutical carrier” includes mixtures of two or more such carriers, and the like.

Ranges are often expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment.

This disclosure includes information that may be useful in understanding the present invention. It is not an admission that any of the information provided herein is prior art or relevant to the presently claimed invention, or that any publication specifically or implicitly referenced is prior art.

Treatment of Cancer

The treatment of cancer remains a significant challenge. While “cancers” share many characteristics in common, each particular cancer has its own specific characteristics. Genetics and environmental factors have a complex interplay in severity and prognosis of treatment. Thus, treatment must be carefully tailored. Certain pharmaceutical treatments have proven useful for one form of cancer, but not others. Other treatments such as radiation, while partially useful for a range of cancers, do not typically result in a complete cure. Indeed, given the severity of many cancers and the mortality rate, a drug can be deemed successful if it improves quality of life, e.g., by delaying growth of tumours, or prolongs life, without actually curing the condition. Thus, in many circumstances, an individual is treated with a compound or combination of treatments that can eliminate 90-95% of the malignant cells, but the remaining cells can regrow and metastasize, ultimately resulting in death.

Certain macrolide compounds have been suggested for use in the treatment of cancer (WO07110704, WO07110705 and EP1380579A). However, there remains a need to provide active compounds, particularly those with good pharmacokinetic properties such as solubility and stability (especially stability in solution) that can be used in the treatment of proliferative conditions such as cancers, in particular solid cancers, and other related disorders.

### SUMMARY OF THE INVENTION

One aspect of the invention pertains to certain macrolide compounds (for convenience, collectively referred to herein as “MC compounds”), as described herein.

Another aspect of the invention pertains to a composition (e.g., a pharmaceutical composition) comprising a MC compound, as described herein, and a pharmaceutically acceptable carrier or diluent.

Another aspect of the invention pertains to method of preparing a composition (e.g., a pharmaceutical composition) comprising the step of admixing a MC compound, as described herein, and a pharmaceutically acceptable carrier or diluent.

Another aspect of the present invention pertains to a method of treatment comprising administering to a subject in need of treatment a therapeutically-effective amount of a MC compound, as described herein, preferably in the form of a pharmaceutical composition.

In one embodiment, the method further comprises administering to the subject one or more other agents selected from: (a) a DNA topoisomerase I or II inhibitor; (b) a DNA damaging agent; (c) an antimetabolite or TS inhibitor; (d) a microtubule targeted agent; and (e) ionising radiation.

Another aspect of the present invention pertains to a MC compound as described herein for use in a method of treatment of the human or animal body by therapy.

In one embodiment, the method of treatment comprises treatment with both (i) a MC compound and (ii) one or more other agents selected from: (a) a DNA topoisomerase I or II inhibitor; (b) a DNA damaging agent; (c) an antimetabolite or TS inhibitor; (d) a microtubule targeted agent; and (e) ionising radiation.

In one embodiment, the treatment is treatment of a proliferative condition.

In one embodiment, the treatment is treatment of cancer.

In one embodiment, the treatment is treatment of a solid cancer.

Another aspect of the present invention pertains to use of a MC compound, as described herein, in the manufacture of a medicament for use in treatment.

In one embodiment, the treatment comprises treatment with both (i) a medicament comprising a MC compound and (ii) one or more other agents selected from: (a) a DNA topoisomerase I or II inhibitor; (b) a DNA damaging agent; (c) an antimetabolite or TS inhibitor; (d) a microtubule targeted agent; and (e) ionising radiation.

Another aspect of the present invention pertains to a method of treating a cell, in vitro or in vivo, comprising contacting the cell with an effective amount of a MC compound, as described herein.

In one embodiment, the method further comprises contacting the cell with one or more other agents selected from: (a) a DNA topoisomerase I or II inhibitor; (b) a DNA damaging agent; (c) an antimetabolite or TS inhibitor; (d) a microtubule targeted agent; and (e) ionising radiation.

Another aspect of the present invention pertains to a method of regulating (e.g., inhibiting) cell proliferation (e.g., proliferation of a cell) and/or promoting apoptosis, in vitro or in vivo, comprising contacting a cell with an effective amount of a MC compound, as described herein.

In one embodiment, the method further comprises contacting the cell with one or more other agents selected from: (a) a DNA topoisomerase I or II inhibitor; (b) a DNA damaging agent; (c) an antimetabolite or TS inhibitor; (d) a microtubule targeted agent; and (e) ionising radiation.

Another aspect of the present invention pertains to a method of treating a disease or condition that is ameliorated by regulation (e.g., inhibition) of cell proliferation (e.g., proliferation of a cell) and/or promotion of apoptosis, comprising administering to a subject in need of treatment a therapeutically-effective amount of a MC compound, as described herein, preferably in the form of a pharmaceutical composition.

Another aspect of the present invention pertains to a kit comprising (a) a MC compound, as described herein, preferably provided as a pharmaceutical composition and in a suitable container and/or with suitable packaging; and (b) instructions for use, for example, written instructions on how to administer the compound.

In one embodiment, the kit further comprises one or more other agents selected from: (a) a DNA topoisomerase I or II inhibitor; (b) a DNA damaging agent; (c) an antimetabolite or TS inhibitor; and (d) a microtubule targeted agent.

Another aspect of the present invention pertains to a MC compound obtainable by a method of synthesis as described herein, or a method comprising a method of synthesis as described herein.

Another aspect of the present invention pertains to a MC compound obtained by a method of synthesis as described herein, or a method comprising a method of synthesis as described herein.

Another aspect of the present invention pertains to novel intermediates, as described herein, which are suitable for use in the methods of synthesis described herein.

Another aspect of the present invention pertains to the use of such novel intermediates, as described herein, in the methods of synthesis described herein.

As will be appreciated by one of skill in the art, features and preferred embodiments of one aspect of the invention will also pertain to other aspect of the invention.

### DETAILED DESCRIPTION OF THE INVENTION

Compounds

One aspect of the present invention relates to certain macrolide compounds having a twelve-membered lactone macrocycle and a side chain adjacent the lactone functionality (for convenience, collectively referred to herein as “MC compounds”).

In one embodiment, the compounds are selected from compounds of the following formula, and pharmaceutically acceptable salts, hydrates, and solvates thereof:

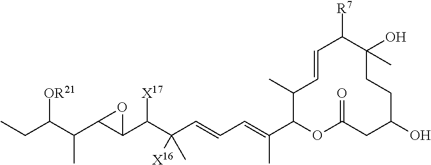

wherein:
—R⁷ is independently —OH, —OR^{7A}, or —O—C(O)R^{7A}
   wherein:
   —R^{7A} is independently —R^{7A1}, —R^{7A2} or —R^{7A3}
      wherein:
      —R^{7A1} is independently saturated or unsaturated aliphatic C₁₋₆alkyl, and is optionally substituted
      and wherein:
      —R^{7A2} is independently saturated or unsaturated alicyclic or aromatic C₃₋₂₀-carbocyclyl, and is optionally substituted
      and wherein:
      —R^{7A3} is independently —NH₂, —NHR^{7NA}, —N(R^{7NA})₂, or —NR^{7NB}R^{7NC}
         wherein:
         each R^{7NA} is independently —R^{7NA1} or —R^{7NA2}
         wherein:
         —R^{7NA1} is independently saturated or unsaturated aliphatic C₁₋₆alkyl, and is optionally substituted
         and wherein:
         —R^{7NA2} is independently saturated or unsaturated alicyclic or aromatic, carbo or hetero, C₃₋₂₀cyclyl, and is optionally substituted
      and wherein:
      —NR^{7NB}R^{7NC} is independently saturated or unsaturated alicyclic or aromatic C₃₋₂₀heterocyclyl, and is optionally substituted
         and wherein:
—X¹⁶ is independently —OR^{16A} or —NR^{16A}R^{16B}
   wherein:
   R^{16A} is independently —H, —Z^{16A}, —Y^{16A}Z^{16A} or together with —R^{17A} is -L-
      wherein:
      —Y^{16A}— is independently saturated or unsaturated aliphatic C₁₋₄ alkylene, and is optionally substituted
      and wherein:
      -L- is independently saturated or unsaturated aliphatic C₁₋₄ alkylene, —S(O)(O)— or —C(O)—, and is optionally substituted
      and wherein:
      —Z^{16A} is independently —Z^{16A1} or —Z^{16A2}
         wherein:
         —Z^{16A1} is independently saturated or unsaturated aliphatic C₁₋₆alkyl, and is optionally substituted
         and wherein:
         —Z^{16A2} is independently saturated or unsaturated alicyclic or aromatic C₃₋₂₀cyclyl, and is optionally substituted
   and wherein:
   R^{16B} is independently —H, —Z^{16B} or —Y^{16B}Z^{16B}
      wherein:
      —Y^{16B} is independently saturated or unsaturated aliphatic C₁₋₄ alkylene, and is optionally substituted
      and wherein:
      —Z^{16B} is independently —Z^{16B1} or —Z^{16B2}
         wherein:
         —Z^{16B1} is independently saturated or unsaturated aliphatic C₁₋₆alkyl, and is optionally substituted
         and wherein:
         —Z^{16B2} is independently saturated or unsaturated alicyclic or aromatic C₃₋₂₀cyclyl, and is optionally substituted
            and wherein:
—X¹⁷ is independently —NR^{17A}R^{17B}
wherein:
   —R^{17A} is independently —H, —Z^{17A}, —Y^{17A}Z^{17A} or together with —R^{16A} is -L-
      wherein:
      —Y^{17A}— is independently saturated or unsaturated aliphatic C₁₋₄ alkylene, —C(O)—, or —S(O)(O)—, and is optionally substituted
      and wherein:
      -L- is independently as defined above
      and wherein:
      —Z^{17A} is independently —Z^{17A1} or —Z^{17A2}
         wherein:
         —Z^{17A1} is independently saturated or unsaturated aliphatic C₁₋₆alkyl, and is optionally substituted
         and wherein:
         —Z^{17A2} is independently —Z^{17AH} or —Z^{17AC}
         wherein:
         —Z^{17AH} is saturated or unsaturated alicyclic or aromatic C₃₋₂₀heterocyclyl, and is optionally substituted
         and wherein:
         —Z^{17AC} is saturated or unsaturated alicyclic or aromatic C₃₋₂₀carbocyclyl, and is optionally substituted
and wherein:
   —R^{17B} is independently —H, —Z^{17B} or —Y^{17B}Z^{17B},
      wherein:
      —Y^{17B}— is independently saturated or unsaturated aliphatic C₁₋₄ alkylene, —C(O)—, or —S(O)(O)—, and is optionally substituted
      and wherein:
      —Z^{17B} is independently —Z^{17B1}, —Z^{17B2} or —Z^{17B3}.
         wherein:
         —Z^{17B1} is independently saturated or unsaturated aliphatic C₁₋₆alkyl, and is optionally substituted
         and wherein:
         —Z^{17B2} is independently saturated or unsaturated alicyclic or aromatic C₃₋₂₀carbocyclyl, and is optionally substituted
         and wherein:
         —Z^{17B3} is independently saturated or unsaturated alicyclic or aromatic C₃₋₂₀heterocyclyl, and is optionally substituted
            and wherein:
—R²¹ is independently —H or -Me.

For the avoidance of doubt, the indices such as “C₅₋₆” and “C₄₋₇” in terms such as “C₃₋₂₀cyclyl” refer to the number of ring atoms, whether carbon atoms or heteroatoms. For example, cyclohexyl, piperidyl, pyridinyl and piperazyl are example of a C₆cyclyl group.

For the avoidance of doubt, it is not intended that the groups —R⁶ and —R⁷ are linked, other than via the macrocycle to which they are attached.

The Group —R⁷

Optionally, —R⁷ is —OH, —OR^{7A}, or —O—C(O)R^{7A}.

Optionally, —R⁷ is —O—C(O)R^{7A} or —OH.

Optionally, —R⁷ is —OH.

Optionally, —R⁷ is —OR^{7A}.

Optionally, —R⁷ is —O—C(O)R^{7A}.

The Group —R^{7A}

Optionally, —R^{7A}, if present, is independently —R^{7A1}, —R^{7A2} or —R^{7A3}.

Optionally, —R^{7A}, if present, is independently —R^{7A1}.

Optionally, —R^{7A}, if present, is independently —R^{7A2}.

Optionally, —R^{7A}, if present, is independently —R^{7A3}.

The Group —R^{7A1}

Optionally, —R^{7A1}, if present, is independently saturated or unsaturated aliphatic C₁₋₆alkyl, and is optionally substituted.

Optionally, —R^{7A1}, if present, is independently saturated or unsaturated aliphatic C₁₋₃alkyl, and is optionally substituted.

Optionally, —R^{7A1}, if present, is independently saturated or unsaturated aliphatic C₁₋₂alkyl, and is optionally substituted.

Optionally, —R^{7A1}, if present, is independently -Me.

The Group —R^{7A2}

Optionally, —R^{7A2}, if present, is independently saturated or unsaturated alicyclic or aromatic C₃₋₂₀carbocyclyl, and is optionally substituted.

Optionally, —R^{7A2}, if present, is independently saturated or unsaturated alicyclic or aromatic C₃₋₁₅carbocyclyl, and is optionally substituted.

Optionally, —R^{7A2}, if present, is independently saturated or unsaturated alicyclic or aromatic C₄₋₁₀carbocyclyl, and is optionally substituted.

Optionally, —R^{7A2}, if present, is independently saturated or unsaturated alicyclic or aromatic C₅₋₇carbocyclyl, and is optionally substituted.

Optionally, —R^{7A2}, if present, is independently saturated or unsaturated alicyclic C₅₋₇carbocyclyl or C₅₋₇carboaryl, and is optionally substituted.

The Group —R^{7A3}

Optionally, —R^{7A3}, if present, is independently —NH₂, —NHR^{7NA}, —N(R^{7NA})₂, or —NR^{7NB}R^{7NC}.

Optionally, —R^{7A3}, if present, is independently —NH₂.

Optionally, —R^{7A3}, if present, is independently —NHR^{7NA}.

Optionally, —R^{7A3}, if present, is independently —N(R^{7NA})₂.

Optionally, —R^{7A3}, if present, is independently —NR^{7NB}R^{7NC}.

The Group —R^{7NA}

Optionally, —R^{7NA}, if present, is independently —R^{7N1} or —R^{7N2}.

Optionally, —R^{7NA}, if present, is independently —R^{7N1}.

Optionally, —R^{7NA}, if present, is independently —R^{7NA2}.

The Group —R^{7NA1}

Optionally, —R^{7NA1}, if present, is independently saturated or unsaturated aliphatic C₁₋₆alkyl, and is optionally substituted.

Optionally, —R^{7NA1}, if present, is independently saturated or unsaturated aliphatic C₁₋₃alkyl and is optionally substituted.

Optionally, —R^{7NA1}, if present, is independently saturated or unsaturated aliphatic C₁₋₂alkyl and is optionally substituted.

Optionally, —R^{7NA1}, if present, is independently -Me.

The Group —R^{7NA2}

Optionally, —R^{7NA2}, if present, is independently saturated or unsaturated alicyclic or aromatic, carbo or hetero, C₃₋₂₀cyclyl, and is optionally substituted.

Optionally, —R^{7NA2}, if present, is independently saturated or unsaturated alicyclic or aromatic, carbo or hetero, C₃₋₁₅cyclyl, and is optionally substituted.

Optionally, —R^{7NA2}, if present, is independently saturated or unsaturated alicyclic or aromatic, carbo or hetero, C₄₋₁₀cyclyl, and is optionally substituted.

Optionally, —R^{7NA2}, if present, is independently saturated or unsaturated alicyclic or aromatic, carbo or hetero, C₅₋₇cyclyl, and is optionally substituted.

Optionally, —R^{7NA2}, if present, is independently saturated or unsaturated alicyclic, carbo or hetero, C₅₋₇cyclyl or C₅₋₇aryl, and is optionally substituted.

The Group —NR^{7NB}R^{7NC}

Optionally, —NR^{7NB}R^{7NC}, if present, is independently saturated or unsaturated alicyclic or aromatic C₃₋₂₀heterocyclyl, and is optionally substituted.

Optionally, —NR^{7NB}R^{7NC}, if present, is independently saturated or unsaturated alicyclic or aromatic C₃₋₁₅heterocyclyl, and is optionally substituted.

Optionally, —NR^{7NB}R^{7NC}, if present, is independently saturated or unsaturated alicyclic or aromatic C₄₋₁₀heterocyclyl, and is optionally substituted.

Optionally, —NR^{7NB}R^{7NC}, if present, is independently saturated or unsaturated alicyclic or aromatic C₅₋₇heterocyclyl, and is optionally substituted.

Optionally, —NR^{7NB}R^{7NC}, if present, is independently saturated or unsaturated alicyclic C₅₋₇heterocyclyl or C₅₋₇heteroaryl, and is optionally substituted.

Optionally, —NR^{7NB}R^{7NC}, if present, is independently azetidino, diazetidino, pyrrolo, pyrrolino, pyrrolidino, imidazolo, imidazolidino, pyrazolo, pyrazolidino, triazolo, pyridino, piperidino, morpholino, pyridazino, piperazino, pyrimidino, pyrazino, azepino or diazepino and is optionally substituted.

Optionally, —NR^{7NB}R^{7NC}, if present, is independently piperazino, and is optionally substituted.

Optionally, —NR^{7NB}R^{7NC}, if present, is independently:

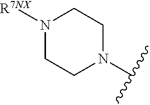

wherein —R^{7NX}, if present, is independently —R^{7NX1} or —R^{7NX2}, wherein:
—R^{7NX1}, if present, is independently saturated or unsaturated aliphatic C₁₋₆alkyl, and is optionally substituted; and
—R^{7NX2}, if present, is independently or saturated or unsaturated alicyclic or aromatic, carbo or hetero, C₃₋₂₀cyclyl and is optionally substituted.

The Group —R^{7NX}

Optionally, —R^{7NX}, if present, is independently —R^{7NX1} or —R^{7NX2}.

Optionally, —R^{7NX}, if present, is independently —R^{7NX1}.

Optionally, —R^{7NX}, if present, is independently —R^{7NX2}.

The Group —R^{7NX1}

Optionally, —R^{7NX1}, if present, is independently saturated or unsaturated aliphatic C₁₋₆alkyl, and is optionally substituted.

Optionally, —R^{7NX1}, if present, is independently saturated or unsaturated aliphatic C₁₋₄alkyl, and is optionally substituted.

Optionally, R^{7NX1}, if present, is independently saturated aliphatic C₁₋₄ alkyl.

The Group —R^{7NX2}

Optionally, —R^{7NX2}, if present, is independently or saturated or unsaturated alicyclic or aromatic, carbo or hetero, C₃₋₂₀cyclyl and is optionally substituted.

Optionally, —R^{7NX2}, if present, is independently saturated or unsaturated alicyclic or aromatic, carbo or hetero, C₃₋₁₅cyclyl and is optionally substituted.

Optionally, —R^{7NX2}, if present, is independently saturated or unsaturated alicyclic or aromatic, carbo or hetero, C₄₋₁₀cyclyl and is optionally substituted.

Optionally, —R^{7NX2}, if present, is independently saturated or unsaturated alicyclic or aromatic, carbo or hetero, C₅₋₇cyclyl and is optionally substituted.

Optionally, —R^{7NX2}, if present, is independently or saturated or unsaturated alicyclic or aromatic C₃₋₂₀carbocyclyl and is optionally substituted.

Optionally, —R^{7NX2}, if present, is independently saturated or unsaturated alicyclic or aromatic C₃₋₁₅carbocyclyl and is optionally substituted.

Optionally, —R^{7NX2}, if present, is independently saturated or unsaturated alicyclic or aromatic C₄₋₁₀carbocyclyl and is optionally substituted.

Optionally, —R^{7NX2}, if present, is independently saturated or unsaturated alicyclic or aromatic C₅₋₇carbocyclyl and is optionally substituted.

Optionally, —R^{7NX2}, if present, is independently cycloheptyl, and is optionally substituted.

The Group —X¹⁶

Optionally, —X¹⁶ is independently —OR^{16A} or —NR^{16A}R^{16B}.

Optionally, —X¹⁶ is independently —OR^{16A}.

Optionally, —X¹⁶ is independently —NR^{16A}R^{16B}.

The Group —R^{16A}

Optionally, R^{16A} is independently —H, —Z¹⁶, —Y^{16A}Z^{16A} or together with —R^{17A} is -L-.

Optionally, R^{16A} is independently —H.

Optionally, R^{16A} is independently —Z^{16A}.

Optionally, R^{16A} is independently —Y^{16A}Z^{16A}.

Optionally, R^{16A} is independently together with —R^{17A} is -L-.

The Group —Y^{16A}

Optionally, —Y^{16A}, if present, is independently saturated or unsaturated aliphatic C₁₋₄alkylene, and is optionally substituted.

Optionally, —Y^{16A}, if present, is independently saturated or unsaturated aliphatic C₁₋₃alkylene, and is optionally substituted.

Optionally, —Y^{16A}, if present, is independently saturated or unsaturated C₁₋₂alkylene, and is optionally substituted.

Optionally, —Y^{16A}—, if present, is independently C₁alkylene, and is optionally substituted.

Optionally, —Y^{16A}—, if present, is independently —CH₂—.

The Group -L-

Optionally, -L-, if present, is independently saturated or unsaturated aliphatic C₁₋₄ alkylene, —S(O)(O)— or —C(O)—, and is optionally substituted.

Optionally, -L-, if present, is independently saturated or unsaturated aliphatic C₁₋₄ alkylene, and is optionally substituted.

Optionally, -L-, if present, is independently —S(O)(O)—.

Optionally, -L-, if present, is independently —C(O)—.

Optionally, -L-, if present, is independently saturated or unsaturated aliphatic C₁₋₃alkylene, and is optionally substituted.

Optionally, -L-, if present, is independently saturated or unsaturated aliphatic C₁₋₂alkylene, and is optionally substituted.

Optionally, -L-, if present, is independently C₂alkylene, and is optionally substituted.

Optionally, -L-, if present, is independently —CH₂—CH₂— or —CH(Ph)—CH(Ph)—.

Optionally, -L-, if present, is independently —CH₂—CH₂—.

Optionally, -L-, if present, is independently —CH(Ph)—CH(Ph)—.

The Group —Z^{16A}

Optionally, —Z^{16A}, if present, is independently —Z^{16A1} or —Z^{16A2}.

Optionally, —Z^{16A}, if present, is independently —Z^{16A1}.

Optionally, —Z^{16A}, if present, is independently —Z^{16A2}.

The Group —Z^{16A1}

Optionally, —Z^{16A1}, if present, is independently saturated or unsaturated aliphatic C₁₋₆alkyl, and is optionally substituted.

Optionally, —Z^{16A1}, if present, is independently saturated or unsaturated aliphatic C₁₋₃alkyl, and is optionally substituted.

Optionally, —Z^{16A1}, if present, is independently saturated or unsaturated C₁₋₂alkyl, and is optionally substituted.

Optionally, —Z^{16A1}, if present, is independently C₁alkyl, and is optionally substituted.

Optionally, —Z^{16A1}, if present, is independently -Me.

The Group —Z^{16A2}

Optionally, —Z^{16A2}, if present, is independently saturated or unsaturated alicyclic or aromatic C₃₋₂₀cyclyl, and is optionally substituted.

Optionally, —Z^{16A2}, if present, is independently saturated or unsaturated alicyclic or aromatic C₃₋₁₅cyclyl, and is optionally substituted.

Optionally, —Z^{16A2}, if present, is independently saturated or unsaturated alicyclic or aromatic C₄₋₁₀cyclyl, and is optionally substituted.

Optionally, —Z^{16A2}, if present, is independently saturated or unsaturated alicyclic or aromatic C₅₋₇cyclyl, and is optionally substituted.

Optionally, —Z^{16A2}, if present, is independently saturated or unsaturated alicyclic or aromatic C₆cyclyl, and is optionally substituted.

Optionally, —Z^{16A2}, if present, is independently saturated or unsaturated alicyclic or aromatic C₃₋₂₀carbocyclyl, and is optionally substituted.

Optionally, —Z^{16A2}, if present, is independently saturated or unsaturated alicyclic or aromatic C₃₋₁₅carbocyclyl, and is optionally substituted.

Optionally, —Z^{16A2}, if present, is independently saturated or unsaturated alicyclic or aromatic C₄₋₁₀carbocyclyl, and is optionally substituted.

Optionally, —Z^{16A2}, if present, is independently saturated or unsaturated alicyclic or aromatic C₅₋₇carbocyclyl, and is optionally substituted.

Optionally, —Z^{16A2}, if present, is independently saturated or unsaturated alicyclic or aromatic C₆carbocyclyl, and is optionally substituted.

Optionally, —Z^{16A2}, if present, is independently saturated or unsaturated alicyclic C₅₋₇carbocyclyl or C₅₋₇carboaryl, and is optionally substituted.

Optionally, —Z^{16A2}, if present, is independently phenyl, and is optionally substituted.

The Group —R^{16B}

Optionally, —R^{16B}, if present, is independently —H, —Z^{16B} or —Y^{16B}Z^{16B}.

Optionally, —R^{16B}, if present, is independently —H.

Optionally, —R^{16B}, if present, is independently —Z^{16B}.

Optionally, —R^{16B}, if present, is independently —Y^{16B}Z^{16B}.

The Group —Y^{16B}

Optionally, —Y^{16B}—, if present, is independently saturated or unsaturated aliphatic C₁₋₄alkylene, and is optionally substituted.

Optionally, —Y^{16B}, if present, is independently saturated or unsaturated aliphatic C₁₋₃alkylene, and is optionally substituted.

Optionally, —Y^{16B}—, if present, is independently saturated or unsaturated C₁₋₂alkylene, and is optionally substituted.

Optionally, —Y^{16B}—, if present, is independently C₁alkylene, and is optionally substituted.

Optionally, —Y^{16B}—, if present, is independently —CH₂—.

The Group —Z^{16B}

Optionally, —Z^{16B}, if present, is independently —Z^{16B1} or —Z^{16B2}.

Optionally, —Z^{16B}, if present, is independently —Z^{16B1}.

Optionally, —Z^{16B}, if present, is independently —Z^{16B2}.

The Group —Z^{16B1}

Optionally, —Z^{16B1}, if present, is independently saturated or unsaturated aliphatic C₁₋₆alkyl, and is optionally substituted.

Optionally, —Z^{16B1}, if present, is independently saturated or unsaturated aliphatic C₁₋₃alkyl, and is optionally substituted.

Optionally, —Z^{16B1}, if present, is independently saturated or unsaturated C₁₋₂alkyl, and is optionally substituted.

Optionally, —Z^{16B1}, if present, is independently C₁alkyl, and is optionally substituted.

Optionally, —Z^{16B1}, if present, is independently -Me.

The Group —Z^{16B2}

Optionally, —Z^{16B2}, if present, is independently saturated or unsaturated alicyclic or aromatic C₃₋₂₀cyclyl, and is optionally substituted.

Optionally, —Z^{16B2}, if present, is independently saturated or unsaturated alicyclic or aromatic C₃₋₁₅cyclyl, and is optionally substituted.

Optionally, —Z^{16B2}, if present, is independently saturated or unsaturated alicyclic or aromatic C₄₋₁₀cyclyl, and is optionally substituted.

Optionally, —Z^{16B2}, if present, is independently saturated or unsaturated alicyclic or aromatic C₅₋₇cyclyl, and is optionally substituted.

Optionally, —Z^{16B2}, if present, is independently saturated or unsaturated alicyclic or aromatic C₆cyclyl, and is optionally substituted.

Optionally, —Z^{16B2}, if present, is independently saturated or unsaturated alicyclic or aromatic C₃₋₂₀carbocyclyl, and is optionally substituted.

Optionally, —Z^{16B2}, if present, is independently saturated or unsaturated alicyclic or aromatic C₃₋₁₅carbocyclyl, and is optionally substituted.

Optionally, —Z^{16B2}, if present, is independently saturated or unsaturated alicyclic or aromatic C₄₋₁₀carbocyclyl, and is optionally substituted.

Optionally, —Z^{16B2}, if present, is independently saturated or unsaturated alicyclic or aromatic C₅₋₇carbocyclyl, and is optionally substituted.

Optionally, —Z^{16B2}, if present, is independently saturated or unsaturated alicyclic or aromatic C₆carbocyclyl, and is optionally substituted.

Optionally, —Z^{16B2}, if present, is independently aturated or unsaturated alicyclic C₅₋₇carbocyclyl or C₅₋₇carboaryl, and is optionally substituted.

Optionally, —Z^{16B2}, if present, is independently phenyl, and is optionally substituted.

The Group —X¹⁷

Optionally, —X¹⁷ is independently —NR^{17A}R^{17B}.

The Group —R^{17A}

Optionally, R^{17A} is independently —H, —Z^{17A}, —Y^{17A}Z^{17A} or together with —R^{16A} is -L-, and is optionally substituted.

Optionally, R^{17A} is independently —H.

Optionally, R^{17A} is independently —Z^{17A}, and is optionally substituted.

Optionally, R^{17A} is independently —Y^{17A}Z^{17A}, and is optionally substituted.

Optionally, R^{17A} is independently together with —R^{16A} is -L-, and is optionally substituted.

The Group —Y^{17A}—

Optionally, —Y^{17A}—, if present, is independently saturated or unsaturated aliphatic C₁₋₄alkylene, —C(O)—, or —S(O)(O)—, and is optionally substituted.

Optionally, —Y^{17A}—, if present, is independently saturated or unsaturated aliphatic C₁₋₄alkylene and is optionally substituted.

Optionally, —Y^{17A}—, if present, is independently —C(O)—.

Optionally, —Y^{17A}—, if present, is independently —S(O)(O)—.

Optionally, —Y^{17A}—, if present, is independently saturated or unsaturated aliphatic C₁₋₄alkylene, and is optionally substituted.

Optionally, —Y^{17A}—, if present, is independently saturated or unsaturated aliphatic C₁₋₃alkylene, and is optionally substituted.

Optionally, —Y^{17A}—, if present, is independently saturated or unsaturated C₁₋₂alkylene, and is optionally substituted.

Optionally, —Y^{17A}—, if present, is independently C₁alkylene, and is optionally substituted.

Optionally, —Y^{17A}, if present, is independently —CH₂—, —C(CH₃)H—, —CH₂—CH₂— or —CH₂—CH₂—CH₂—.

Optionally, —Y^{17A}—, if present, is independently —CH₂—.

Optionally, —Y^{17A}—, if present, is independently —C(CH₃)H—.

Optionally, —Y^{17A}—, if present, is independently —CH₂—CH₂—.

Optionally, —Y^{17A}—, if present, is independently —CH₂—CH₂—CH₂—.

The Group -L-

Optionally, if present, -L- is independently as defined above.

The Group —Z^{17A}

Optionally, if present, is independently —Z^{17A1} or —Z^{17A2}.

Optionally, —Z^{17A}—, if present, is independently —Z^{17A1}.

Optionally, —Z^{17A}—, if present, is independently —Z^{17A2}.

The Group —Z^{17A1}

Optionally, —Z^{17A1}, if present, is independently saturated or unsaturated aliphatic C₁₋₆alkyl, and is optionally substituted.

Optionally, —Z^{17A1}, if present, is independently saturated or unsaturated aliphatic C₁₋₄ alkyl, and is optionally substituted.

Optionally, —Z^{17A1}, if present, is independently saturated or unsaturated aliphatic C₁₋₃alkyl, and is optionally substituted.

Optionally, —Z^{17A1}, if present, is independently saturated or unsaturated C₁₋₃alkyl, and is optionally substituted.

Optionally, —Z^{17A1}, if present, is independently methyl, ethyl or propyl and is optionally substituted.

Optionally, —Z^{17A1}, if present, is independently methyl, and is optionally substituted.

Optionally, —Z^{17A1}, if present, is independently ethyl and is optionally substituted.

Optionally, —Z^{17A1}, if present, is independently propyl and is optionally substituted.

The Group —Z^{17A2}

Optionally, —Z^{17A2}, if present, is independently —Z^{17AH} or —Z^{17AC}.

The Group —Z^{17AH}

Optionally, —Z^{17AH}, if present, is independently saturated or unsaturated alicyclic or aromatic C₃₋₂₀heterocyclyl, and is optionally substituted.

Optionally, —Z^{17AH}, if present, is independently saturated or unsaturated alicyclic or aromatic C₃₋₁₅heterocyclyl, and is optionally substituted.

Optionally, —Z^{17AH}, if present, is independently saturated or unsaturated alicyclic or aromatic C₄₋₁₀heterocyclyl, and is optionally substituted.

Optionally, —Z^{17AH}, if present, is independently saturated or unsaturated alicyclic or aromatic C₅₋₇heterocyclyl, and is optionally substituted.

Optionally, —Z^{17AH}, if present, is independently saturated or unsaturated alicyclic or aromatic C₆heterocyclyl, and is optionally substituted.

Optionally, —Z^{17AH}, if present, is independently saturated or unsaturated alicyclic C₅₋₇heterocyclyl or C₅₋₇heteroaryl, and is optionally substituted.

Optionally, —Z^{17AH}, if present, is independently piperazino or morpholino, and is optionally substituted.

Optionally, —Z^{17AH}, if present, is independently piperazino, and is optionally substituted.

Optionally, —Z^{17AH}, if present, is independently morpholino, and is optionally substituted.

Optionally, —Z^{17AH}, if present, is independently pyridinyl, indolyl, pyrazinyl, pyrrolidinyl, imidazolyl, pyrazolyl, triazolyl, tetrazolyl, furanyl and thiophenyl, and is optionally substituted.

Optionally, —Z^{17AH}, if present, is independently pyridinyl, or indolyl, and is optionally substituted.

Optionally, —Z^{17AH}, if present, is independently pyridinyl, and is optionally substituted.

Optionally, —Z^{17AH}, if present, is independently pyridine-2-yl, pyridine-3-yl or pyridine-4-yl, and is optionally substituted.

Optionally, —Z^{17AH}, if present, is independently indolyl, and is optionally substituted.

Optionally, —Z^{17AH}, if present, is independently indol-5-yl, and is optionally substituted.

The Group —Z^{17AC}

Optionally, —Z^{17AC}, if present, is independently saturated or unsaturated alicyclic or aromatic C₃₋₂₀carbocyclyl, and is optionally substituted.

Optionally, —Z^{17AC}, if present, is independently saturated or unsaturated alicyclic or aromatic C₃₋₁₅carbocyclyl, and is optionally substituted.

Optionally, —Z^{17AC}, if present, is independently saturated or unsaturated alicyclic or aromatic C₄₋₁₀carbocyclyl, and is optionally substituted.

Optionally, —Z^{17AC}, if present, is independently saturated or unsaturated alicyclic or aromatic C₅₋₇carbocyclyl, and is optionally substituted.

Optionally, —Z^{17AC}, if present, is independently saturated or unsaturated alicyclic or aromatic C₆carbocyclyl, and is optionally substituted.

Optionally, —Z^{17AC}, if present, is independently saturated or unsaturated alicyclic C₅₋₇carbocyclyl or C₆₋₁₀carboaryl, and is optionally substituted.

Optionally, —Z^{17AC}, if present, is independently cyclopropyl, cyclobutyl, cyclohexyl or cycloheptyl, and is optionally substituted.

Optionally, —Z^{17AC}, if present, is independently cyclopropyl, and is optionally substituted.

Optionally, —Z^{17AC}, if present, is independently cyclobutyl, and is optionally substituted.

Optionally, —Z^{17AC}, if present, is independently cyclohexyl, and is optionally substituted.

Optionally, —Z^{17AC}, if present, is independently cycloheptyl, and is optionally substituted.

Optionally, —Z^{17AC}, if present, is independently C₆₋₁₀carboaryl, and is optionally substituted.

Optionally, —Z^{17AC}, if present, is independently C₅₋₇carboaryl, and is optionally substituted.

Optionally, —Z^{17AC}, if present, is independently C₆carboaryl, and is optionally substituted.

Optionally, —Z^{17AC}, if present, is independently phenyl or naphthyl, and is optionally substituted.

Optionally, —Z^{17AC}, if present, is independently phenyl, and is optionally substituted.

Optionally, —Z^{17AC}, if present, is independently naphthyl, and is optionally substituted.

The Group —R^{17B}

Optionally, —R^{17B} is independently —H, —Z^{17B} or —Y^{17B}Z^{17B}.

Optionally, —R^{17B} is independently —H.

Optionally, —R^{17B} is independently —Z^{17B}.

Optionally, —R^{17B} is independently —Y^{17B}Z^{17B}.

The Group —Y^{17B}

Optionally, —Y^{17B}—, if present, is independently saturated or unsaturated aliphatic C₁₋₄alkylene, —C(O)—, or —S(O)(O)—, and is optionally substituted.

Optionally, —Y^{17B}—, if present, is independently saturated or unsaturated aliphatic C₁₋₄alkylene and is optionally substituted.

Optionally, —Y^{17B}—, if present, is independently —C(O)—.

Optionally, —Y^{17B}—, if present, is independently —S(O)(O)—.

Optionally, —Y^{17B}—, if present, is independently saturated or unsaturated aliphatic C₁₋₄alkylene, and is optionally substituted.

Optionally, —Y^{17B}—, if present, is independently saturated or unsaturated aliphatic C₁₋₃alkylene, and is optionally substituted.

Optionally, —Y^{17B}—, if present, is independently saturated or unsaturated C₁₋₂alkylene, and is optionally substituted.

Optionally, —Y^{17B}—, if present, is independently C₁alkylene, and is optionally substituted.

Optionally, —Y^{17B}—, if present, is independently —CH₂—, —C(CH₃)H—, —CH₂—CH₂— or —CH₂—CH₂—CH₂—.

Optionally, —Y^{17B}—, if present, is independently —CH₂—.

Optionally, —Y^{17B}—, if present, is independently —C(CH₃)H—.

Optionally, —Y^{17B}—, if present, is independently —CH₂—CH₂—.

Optionally, —Y^{17B}—, if present, is independently —CH₂—CH₂—CH₂—.

The Group —Z^{17B}

Optionally, —Z^{17B}, if present, is independently —Z^{17B1}, —Z^{17B2} or —Z^{17B3}.

Optionally, —Z^{17B}, if present, is independently —Z^{17B1}.

Optionally, —Z^{17B}, if present, is independently —Z^{17B2}.

Optionally, —Z^{17B}, if present, is independently —Z^{17B3}.

The Group —Z^{17B1}

Optionally, —Z^{17B1}, if present, is independently saturated or unsaturated aliphatic C₁₋₆alkyl, and is optionally substituted.

Optionally, —Z^{17B1}, if present, is independently saturated or unsaturated aliphatic C₁₋₄ alkyl, and is optionally substituted.

Optionally, —Z^{17B1}, if present, is independently saturated or unsaturated aliphatic C₁₋₃alkyl, and is optionally substituted.

Optionally, —Z^{17B1}, if present, is independently saturated aliphatic C₁₋₃alkyl, and is optionally substituted.

Optionally, —Z^{17B1}, if present, is independently methyl, ethyl or propyl and is optionally substituted.

Optionally, —Z^{17B1}, if present, is independently methyl, and is optionally substituted.

Optionally, —Z^{17B1}, if present, is independently ethyl and is optionally substituted.

Optionally, —Z^{17B1}, if present, is independently propyl and is optionally substituted.

The Group —Z^{17B2}

Optionally, —Z^{17B2}, if present, is independently saturated or unsaturated alicyclic or aromatic C₃₋₂₀carbocyclyl, and is optionally substituted.

Optionally, —Z^{17B2}, if present, is independently saturated or unsaturated alicyclic or aromatic C₃₋₁₅carbocyclyl, and is optionally substituted.

Optionally, —Z^{17B2}, if present, is independently saturated or unsaturated alicyclic or aromatic C₄₋₁₀carbocyclyl, and is optionally substituted.

Optionally, —Z^{17B2}, if present, is independently saturated or unsaturated alicyclic or aromatic C₅₋₇carbocyclyl, and is optionally substituted.

Optionally, —Z^{17B2}, if present, is independently saturated or unsaturated alicyclic or aromatic C₆carbocyclyl, and is optionally substituted.

Optionally, —Z^{17B2}, if present, is independently saturated or unsaturated alicyclic C₅₋₇carbocyclyl or C₅₋₇carboaryl, and is optionally substituted.

Optionally, —Z^{17B2}, if present, is independently phenyl, and is optionally substituted.

The Group —Z^{17B3}

Optionally, —Z^{17B3}, if present, is independently saturated or unsaturated alicyclic or aromatic C₃₋₂₀heterocyclyl, and is optionally substituted.

Optionally, —Z^{17B3}, if present, is independently saturated or unsaturated alicyclic or aromatic C₃₋₁₅heterocyclyl, and is optionally substituted.

Optionally, —Z^{17B3}, if present, is independently saturated or unsaturated alicyclic or aromatic C₄₋₁₀heterocyclyl, and is optionally substituted.

Optionally, —Z^{17B3}, if present, is independently saturated or unsaturated alicyclic or aromatic C₅₋₇heterocyclyl, and is optionally substituted.

Optionally, —Z^{17B3}, if present, is independently saturated or unsaturated alicyclic or aromatic C₆heterocyclyl, and is optionally substituted.

Optionally, —Z^{17B3}, if present, is independently saturated or unsaturated alicyclic C₅₋₇heterocyclyl or C₅₋₇heteroaryl, and is optionally substituted.

Optionally, —Z^{17B3}, if present, independently contains at least one nitrogen ring atom.

Optionally, —Z^{17B3}, if present, independently contains one nitrogen ring atom.

Optionally, —Z^{17B3}, if present, is independently pyridinyl, and is optionally substituted.

The Group —R²¹

Optionally, —R²¹ is independently —H or -Me.

Optionally, —R²¹ is independently —H.

Optionally, —R²¹ is independently -Me.

Optional Substituents on —R^{7A1}, —R^{7NA1}, —Z^{16A1}, —Z^{16B1}, —Z^{17A1}, and —Z^{17B1}

In one embodiment, —R^{7A1}, if present, is unsubstituted.

In one embodiment, —R^{7A1}, if present, is optionally substituted with one or more substituents, —R^{S1}.

In one embodiment, —R^{7NA1}, if present, is unsubstituted.

In one embodiment, —R^{7NA1}, if present, is optionally substituted with one or more substituents, —R^{S1}.

In one embodiment, —Z^{16A1}, if present, is unsubstituted.

In one embodiment, —Z^{16A1}, if present, is optionally substituted with one or more substituents, —R^{S1}.

In one embodiment, —Z^{16B1}, if present, is unsubstituted.

In one embodiment, —Z^{16B1}, if present, is optionally substituted with one or more substituents, —R^{S1}.

In one embodiment, —Z^{17A1}, if present, is unsubstituted.

In one embodiment, —Z^{17A1}, if present, is optionally substituted with one or more substituents, —R^{S1}.

In one embodiment, —Z^{17B1}, if present, is unsubstituted.

In one embodiment, —Z^{17B1}, if present, is optionally substituted with one or more substituents, —R^{S1}.

In one embodiment, each —R^{S1}, if present, is independently selected from:
—R^{JA1},
—F, —Cl, —Br, —I,
—CF₃, —OCF₃, —SCF₃,
—OH, -L^{JA}-OH, —O-L^{JA}-OH, —NH-L^{JA}-OH, —NR^{JA1}-L^{JA}-OH,
—OR^{JA1}, -L^{JA}-OR^{JA1}, —O-L^{JA}-OR^{JA1}, —NH-L^{JA}-OR^{JA1}, —NR^{JA1}-L^{JA}-OR^{JA1},
—SH, —SR^{JA1},
—CN,
—NO₂,
—NH₂, —NHR^{JA1}—NR^{JA1}₂, —NR^{JA2}R^{JA3},
-L^{JA}-NH₂, -L^{JA}-NHR^{JA1}, -L^{JA}-NR^{JA1}₂, —O-L^{JA}-NR^{JA2}R^{JA3},
—O-L^{JA}-NH₂, —O-L^{JA}-NHR^{JA1}, —O-L^{JA}-NR^{JA1}₂, —O-L^{JA}-NR^{JA2}R^{JA3},
—NH-L^{JA}-NH₂, —NR^{JA1}-L^{JA}-NH₂, —NH-L^{JA}-NHR^{JA1}, —NR^{JA1}-L^{JA}-NHR^{JA1},
—NH-L^{JA}-NR^{JA1}₂, —NR^{JA1}-L^{JA}-NR^{JA1}₂,
—NH-L^{JA}-NR^{JA2}R^{JA3}, —NR^{JA1}-L^{JA}-NR^{JA2}R^{JA3},
—OC(═O)R^{JA1},
—C(═O)OH, —C(═O)OR^{JA1},
—C(═O)R^{JA1},
—C(═O)NH₂, —C(═O)NHR^{JA1}—C(═O)NR^{JA1}₂, —C(═O)NR^{JA2}R^{JA3},
—NHC(═O)R^{JA1}, —NR^{JA1}C(═O)R^{JA1},
—NHC(═O)OR^{JA1}, —NR^{JA1}C(═O)OR^{JA1},
—OC(═O)NH₂, —OC(═O)NHR^{JA1}, —OC(═O)NR^{JA1}₂, —OC(═O)NR^{JA2}R^{JA3},
—NHC(═O)NH₂, —NHC(═O)NHR^{JA1},
—NHC(═O)NR^{JA1}₂, —NHC(═O)NR^{JA2}R^{JA3},
—NR^{JA1}C(═O)NH₂, —NR^{JA1}C(═O)NHR^{JA1},
—NR^{JA1}C(═O)NR^{JA1}₂, —NR^{JA1}C(═O)NR^{JA2}R^{JA3},
—NHS(═O)₂R^{JA1}, —NR^{JA1}S(═O)₂R^{JA1},
—S(═O)₂NH₂, —S(═O)₂NHR^{JA1}, —S(═O)₂NR^{JA1}₂, —S(═O)₂NR^{JA2}R^{JA3},
—S(═O)R^{JA1}, —S(═O)₂R^{JA1}, —OS(═O)₂R^{JA1}, —S(═O)₂OH, —S(═O)₂OR^{JA1}; and
═O;

In one embodiment, each —R^{S1}, if present, is independently selected from:
—R^{JA1},
—F, —Cl, —Br, —I
—CF₃, —OCF₃,
—OH, -L^{JA}-OH, —O-L^{JA}-OH,
—OR^{JA1}, -L^{JA}-OR^{JA1}, —O-L^{JA}-OR^{JA1},
—CN,
—NH₂, —NHR^{JA1}, —NR^{JA1}₂,
-L^{JA}-NH₂, -L^{JA}-NHR^{JA1}, -L^{JA}-NR^{JA1}₂,
—C(═O)OH, —C(═O)OR^{JA1},
—C(═O)R^{JA1},
—C(═O)NH₂, —C(═O)NHR^{JA1}, —C(═O)NR^{JA1}₂, —C(═O)NR^{JA2}R^{JA3},
—NHC(═O)R^{JA1}, —NR^{JA1}C(═O)R^{JA1},
—NHC(═O)OR^{JA1}, —NR^{JA1}C(═O)OR^{JA1},
—S(═O)R^{JA1}, —S(═O)₂R^{JA1}, —OS(═O)₂R^{JA1}, —S(═O)₂OH, —S(═O)₂OR^{JA1}; and
═O.

In one embodiment, each —R^{S1}, if present, is independently selected from:
R^{JA1},
—F, —Cl, —Br, —I
—CF₃,
—OH, -L^{JA}-OH, —O-L^{JA}-OH,
—OR^{JA1}, -L^{JA}-OR^{JA1}, —O-L^{JA}-OR^{JA1},
—CN,
—NH₂, —NHR^{JA1}, —NR^{JA1}₂, and
—NHC(═O)OR^{JA1}, —NR^{JA1}C(═O)OR^{JA1}.

Optional Substituents on —R^{7A2}, —R^{7NA2}, —Z^{16A2} and —Z^{16B2}.

In one embodiment, —R^{7A2}, if present, is unsubstituted.

In one embodiment, —R^{7A2}, if present, is optionally substituted with one or more substituents, —R^{S2}.

In one embodiment, —R^{7NA2}, if present, is unsubstituted.

In one embodiment, —R^{7NA2}, if present, is optionally substituted with one or more substituents, —R^{S2}.

In one embodiment, —Z^{16A2}, if present, is unsubstituted.

In one embodiment, —Z^{16A2}, if present, is optionally substituted with one or more substituents, —R^{S2}.

In one embodiment, —Z^{16B2}, if present, is unsubstituted.

In one embodiment, —Z^{16B2}, if present, is optionally substituted with one or more substituents, —R^{S2}.

In one embodiment, each —R^{S2}, if present, is independently selected from:
—R^{JA1},
—F, —Cl, —Br, —I,
—CF₃, —OCF₃, —SCF₃,
—OH, -L^{JA}-OH, —O-L^{JA}-OH, —NH-L^{JA}-OH, —NR^{JA1}-L^{JA}-OH,
—OR^{JA1}, -L^{JA}-OR^{JA1}, —O-L^{JA}-OR^{JA1}, —NH-L^{JA}-OR^{JA1}, —NR^{JA1}-L^{JA}-OR^{JA1},
—SH, —SR^{JA1},
—CN,
—NO₂,
—NH₂, —NHR^{JA1}, —NR^{JA1}₂, —NR^{JA2}R^{JA3},
-L^{JA}-NH₂, -L^{JA}-NHR^{JA1}, -L^{JA}-NR^{JA1}₂, -L^{JA}-NR^{JA2}R^{JA3},
—O-L^{JA}-NH₂, —O-L^{JA}-NHR^{JA1}, —O-L^{JA}-NR^{JA1}₂, —O-L^{JA}-NR^{JA2}R^{JA3},
—NH-L^{JA}-NH₂, —NR^{JA1}-L^{JA}-NH₂, —NH-L^{JA}-NHR^{JA1}, —NR^{JA1}-L^{JA}-NHR^{JA1},
—NH-L^{JA}-NR^{JA1}₂, —NR^{JA1}-L^{JA}-NR^{JA1}₂,
—NH-L^{JA}-NR^{JA2}R^{JA3}, —NR^{JA1}-L^{JA}-NR^{JA2}R^{JA3},
—OC(═O)R^{JA1},
—C(═O)OH, —C(═O)OR^{JA1},
—C(═O)R^{JA1},
—C(═O)NH₂, —C(═O)NHR^{JA1}, —C(═O)NR^{JA1}₂, —C(═O)NR^{JA2}R^{JA3},
—NHC(═O)R^{JA1}, —NR^{JA1}C(═O)R^{JA1},
—NHC(═O)OR^{JA1}, —NR^{JA1}C(═O)OR^{JA1},
—OC(═O)NH₂, —OC(═O)NHR^{JA1}, —OC(═O)NR^{JA1}₂, —OC(═O)NR^{JA2}R^{JA3},
—NHC(═O)NH₂, —NHC(═O)NHR^{JA1},
—NHC(═O)NR^{JA1}₂, —NHC(═O)NR^{JA2}R^{JA3},
—NR^{JA1}C(═O)NH₂, —NR^{JA1}C(═O)NHR^{JA1},
—NHC(═O)NR^{JA1}₂, —NHC(═O)NR^{JA2}R^{JA3},
—NHS(═O)₂R^{JA1}, —NR^{JA1}S(═O)₂R^{JA1},
—S(═O)₂NH₂, —S(═O)₂NHR^{JA1}, —S(═O)₂NR^{JA1}₂, —S(═O)₂NR^{JA2}R^{JA3},
—S(═O)R^{JA1}, —S(═O)₂R^{JA1}, —OS(═O)₂R^{JA1}, —S(═O)₂OH, —S(═O)₂OR^{JA1},
═O; and
two adjacent groups —R^{S2}, if present, together form —O—CH₂—O— or —O—CH₂CH₂—O—.

In one embodiment, each —R^{S2}, if present, is independently selected from:
—R^{JA1},
—F, —Cl, —Br, —I—
—CF₃, —OCF₃,
—OH, -L^{JA}-OH, —O-L^{JA}-OH,
—OR^{JA1}, -L^{JA}-OR^{JA1}, —O-L^{JA}-OR^{JA1},
—CN,
—NO₂,
—NH₂, —NHR^{JA1}, —NR^{JA1}₂, —NR^{JA2}R^{JA3},
-L^{JA}-NH₂, -L^{JA}-NHR^{JA1}, -L^{JA}-NR^{JA1}₂, -L^{JA}-NR^{JA2}R^{JA3},
—C(═O)OH, —C(═O)OR^{JA1},
—C(═O)R^{JA1},
—C(═O)NH₂, —C(═O)NHR^{JA1}, —C(═O)NR^{JA1}₂, —C(═O)NR^{JA2}R^{JA3},
—NHC(═O)R^{JA1}, —NR^{JA1}C(═O)R^{JA1},
—S(═O)R^{JA1}, —S(═O)₂R^{JA1}, —OS(═O)₂R^{JA1}, —S(═O)₂OH, —S(═O)₂OR^{JA1};
═O; and
two adjacent groups —R^{S2}, if present, together form —O—CH₂—O— or —O—CH₂CH₂—O—.

In one embodiment, each —R^{S2}, if present, is independently selected from:
—R^{JA1},
—F, —Cl, —Br, —I—
CF₃,
—OH, -L^{JA}-OH,
—OR^{JA1}, -L^{JA}-OR^{JA1},
—CN,
—NH₂, —NHR^{JA1}, —NR^{JA1}₂, —NR^{JA2}R^{JA3},
-L^{JA}-NH₂, -L^{JA}-NHR^{JA1}, -L^{JA}-NR^{JA1}₂,
—C(═O)OH,
—C(═O)R^{JA1},
—C(═O)NH₂, —C(═O)NHR^{JA1}, and —C(═O)NR^{JA1}₂; and
two adjacent groups —R^{S2}, if present, together form —O—CH₂—O— or —O—CH₂CH₂—O—.

Optional Substituents on —NR^{7NB}R^{7NC}, —Z^{16B3} and Z^{17AH}

In one embodiment, —NR^{7NB}R^{7NC}, if present, is unsubstituted.

In one embodiment, —NR^{7NB}R^{7NC}, if present, is optionally substituted with one or more substituents, —R^{S3}.

In one embodiment, —Z^{16B3}, if present, is unsubstituted.

In one embodiment, —Z^{16B3}, if present, is optionally substituted with one or more substituents, —R^{S3}.

In one embodiment, —Z^{17AH}, if present, is unsubstituted.

In one embodiment, —Z^{17AH}, if present, is optionally substituted with one or more substituents, —R^{S3}.

In one embodiment, each —R^{S3}, if present, is independently selected from:
—R^{JA1},
—F, —Cl, —Br, —I,
—CF₃, —OCF₃, —SCF₃,
—OH, -L^{JA}-OH, —O-L^{JA}-OH, —NH-L^{JA}-OH, —NR^{JA1}-L^{JA}-OH,
—OR^{JA1}, -L^{JA}-OR^{JA1}, —O-L^{JA}-OR^{JA1}, —NH-L^{JA}-OR^{JA1}, —NR^{JA1}-L^{JA}-OR^{JA1},
—SH, —SR^{JA1},
—CN,
—NO₂,
—NH₂, —NHR^{JA1}, —NR^{JA1}₂, —NR^{JA2}R^{JA3},
-L^{JA}-NH₂, -L^{JA}-NHR^{JA1}, -L^{JA}-NR^{JA1}₂, -L^{JA}-NR^{JA2}R^{JA3},
—O-L^{JA}-NH₂, —O-L^{JA}-NHR^{JA1}, —O-L^{JA}-NR^{JA1}₂, —O-L-NR^{JA2}R^{JA3},
—NH-L^{JA}-NH₂, —NR^{JA1}-L^{JA}-NH₂, —NH-L^{JA}-NHR^{JA1}, —NR^{JA1}-L^{JA}-NHR^{JA1},
—NH-L^{JA}-NR^{JA1}₂, —NR^{JA1}-L^{JA}-NR^{JA1}₂,
—NH-L^{JA}-NR^{JA2}R^{JA3}, —NR^{JA1}-L^{JA}-NR^{JA2}R^{JA3},
—OC(═O)R^{JA1},
—C(═O)OH, —C(═O)OR^{JA1},
—C(═O)R^{JA1},
—C(═O)NH₂, —C(═O)NHR^{JA1}, —C(═O)NR^{JA1}₂, —C(═O)NR^{JA2}R^{JA3},
—NHC(═O)R^{JA1}, —NR^{JA1}C(═O)R^{JA1},
—NHC(═O)OR^{JA1}, —NR^{JA1}C(═O)OR^{JA1},
—OC(═O)NH₂, —OC(═O)NHR^{JA1}, —OC(═O)NR^{JA1}₂, —OC(═O)NR^{JA2}R^{JA3},
—NHC(═O)NH₂, —NHC(═O)NHR^{JA1},
—NHC(═O)NR^{JA1}₂, —NHC(═O)NR^{JA2}R^{JA3},
—NR^{JA1}C(═O)NH₂, —NR^{JA1}C(═O)NHR^{JA1},
—NR^{JA1}C(═O)NR^{JA1}₂, —NR^{JA1}C(═O)NR^{JA2}R^{JA3},
—NHS(═O)₂R^{JA1}, —NR^{JA1}S(═O)₂R^{JA1},
—S(═O)₂NH₂, —S(═O)₂NHR^{JA1}, —S(═O)₂NR^{JA1}₂, —S(═O)₂NR^{JA2}R^{JA3},
—S(═O)R^{JA1}, —S(═O)₂R^{JA1}—OS(═O)₂R^{JA1}, —S(═O)₂OH, —S(═O)₂OR^{JA1},
═O; and
two adjacent groups —R^{S3}, if present, together form —O—CH₂—O— or —O—CH₂CH₂—O—.

In one embodiment, each —R^{S3}, if present, is independently selected from:
—R^{JA1},
—F, —Cl, —Br, —I—
—CF₃, —OCF₃,
—OH, -L^{JA}-OH, —O-L^{JA}-OH,
—OR^{JA1}, -L^{JA}-OR^{JA1}, —O-L^{JA}-OR^{JA1},
—CN,
—NO₂,
—NH₂, —NHR^{JA1}, —NR^{JA1}₂, —NR^{JA2}R^{JA3},
-L^{JA}-NH₂, -L^{JA}-NHR^{JA1}, -L^{JA}-NR^{JA1}₂, -L^{JA}-NR^{JA2}R^{JA3},
—C(═O)OH, —C(═O)OR^{JA1},
—C(═O)R^{JA1},
—C(═O)NH₂, —C(═O)NHR^{JA1}, —C(═O)NR^{JA1}₂, —C(═O)NR^{JA2}R^{JA3},
—NHC(═O)R^{JA1}, —NR^{JA1}C(═O)R^{JA1},
—S(═O)R^{JA1}, —S(═O)₂R^{JA1}, —OS(═O)₂R^{JA1}, —S(═O)₂OH, —S(═O)₂OR^{JA1};
═O; and
two adjacent groups —R^{S3}, if present, together form —O—CH₂—O— or —O—CH₂CH₂—O—.

In one embodiment, each —R^{S3}, if present, is independently selected from:
—R^{JA1},
—R^{JA1},
—F, —Cl, —Br, —I
—CF₃,
—OH, -L^{JA}-OH,
—OR^{JA1}, -L^{JA}-OR^{JA1},
—CN,
—NH₂, —NHR^{JA1}, —NR^{JA1}₂, —NR^{JA2}R^{JA3},
-L^{JA}-NH₂, -L^{JA}-NHR^{JA1}, -L^{JA}-NR^{JA1}₂,
—C(═O)OH,
—C(═O)R^{JA1},
—C(═O)NH₂, —C(═O)NHR^{JA1}, and —C(═O)NR^{JA1}₂; and
two adjacent groups —R^{S3}, if present, together form —O—CH₂—O— or —O—CH₂CH₂—O—.

Optional Substituents on —Z^{17AC}, —Z^{17B2} and —Z^{17B3}

In one embodiment, —Z^{17AC}, if present, is unsubstituted.

In one embodiment, —Z^{17AC}, if present, is optionally substituted with one or more substituents, —R^{S4}.

In one embodiment, —Z^{17B2}, if present, is unsubstituted.

In one embodiment, —Z^{17B2}, if present, is optionally substituted with one or more substituents, —R^{S4}.

In one embodiment, —Z^{17B3}, if present, is unsubstituted.

In one embodiment, —Z^{17B3}, if present, is optionally substituted with one or more substituents, —R^{S4}.

In one embodiment, each —R^{S4}, if present, is independently selected from:
—R^{JA1},
—F, —Cl, —Br, —I,
—CF₃, —OCF₃, —SCF₃,
—OH, -L^{JA}-OH, —O-L^{JA}-OH, —NH-L^{JA}-OH, —NR^{JA1}-L^{JA}-OH,
—OR^{JA1}, -L^{JA}-OR^{JA1}, —O-L^{JA}-OR^{JA1}, —NH-L^{JA}-OR^{JA1}, —NR^{JA1}-L^{JA}-OR^{JA1},
—SH, —SR^{JA1},
—CN,
—NO₂,
—NH₂, —NHR^{JA1}, —NR^{JA1}₂, —NR^{JA2}R^{JA3},
-L^{JA}-NH₂, -L^{JA}-NHR^{JA1}, -L^{JA}-NR^{JA1}₂, -L^{JA}-NR^{JA2}R^{JA3},
—O-L^{JA}-NH₂, —O-L^{JA}-NHR^{JA1}, —O-L^{JA}-NR^{JA1}₂, —O-L^{JA}-NR^{JA2}R^{JA3},
—NH-L^{JA}-NH₂, —NR^{JA1}-L^{JA}-NH₂, —NH-L^{JA}-NHR^{JA1}, —NR^{JA1}-L^{JA}-NHR^{JA1},
—NH-L^{JA}-NR^{JA1}₂, —NR^{JA1}-L^{JA}-NR^{JA1}₂,
—NH-L^{JA}-NR^{JA2}R^{JA3}, —NR^{JA1}-L^{JA}-NR^{JA2}R^{JA3},
—OC(═O)R^{JA1},
—C(═O)OH, —C(═O)OR^{JA1},
—C(═O)R^{JA1},
—C(═O)NH₂, —C(═O)NHR^{JA1}, —C(═O)NR^{JA1}₂, —C(═O)NR^{JA2}R^{JA3},
—NHC(═O)R^{JA1}, —NR^{JA1}C(═O)R^{JA1},
—NHC(═O)OR^{JA1}, —NR^{JA1}C(═O)OR^{JA1},
—OC(═O)NH₂, —OC(═O)NHR^{JA1}, —OC(═O)NR^{JA1}₂, —OC(═O)NR^{JA2}R^{JA3},
—NHC(═O)NH₂, —NHC(═O)NHR^{JA1},
—NHC(═O)NR^{JA1}₂, —NHC(═O)NR^{JA2}R^{JA3},
—NR^{JA1}C(═O)NH₂, —NR^{JA1}C(═O)NHR^{JA1},
—NR^{JA1}C(═O)NR^{JA1}₂, —NR^{JA1}C(═O)NR^{JA2}R^{JA3},
—NHS(═O)₂R^{JA1}, —NR^{JA1}S(═O)₂R^{JA1},
—S(═O)₂NH₂, —S(═O)₂NHR^{JA1}, —S(═O)₂NR^{JA1}₂, —S(═O)₂NR^{JA2}R^{JA3},
—S(═O)R^{JA1}, —S(═O)₂R^{JA1}, —OS(═O)₂R^{JA1}, —S(═O)₂OH, —S(═O)₂OR^{JA1},
═O; and
two adjacent groups —R^{S4}, if present, together form —O—CH₂—O— or —O—CH₂CH₂—O—.

In one embodiment, each —R^{S4}, if present, is independently selected from:
—R^{JA1},
—F, —Cl, —Br, —I
—CF₃, —OCF₃,
—OH, -L^{JA}-OH, —O-L^{JA}-OH,
—OR^{JA1}, -L^{JA}-OR^{JA1}, —O-L^{JA}-OR^{JA1},
—CN,
—NO₂,
—NH₂, —NHR^{JA1}—NR^{JA1}₂, —NR^{JA2}R^{JA3},
-L^{JA}-NH₂, -L^{JA}-NHR^{JA1}, -L^{JA}-NR^{JA1}₂, -L^{JA}-NR^{JA2}R^{JA3},
—C(═O)OH, —C(═O)OR^{JA1},
—C(═O)R^{JA1},
—C(═O)NH₂, —C(═O)NHR^{JA1}, —C(═O)NR^{JA1}₂, —C(═O)NR^{JA2}R^{JA3},
—NHC(═O)R^{JA1}, —NR^{JA1}C(═O)R^{JA1},
—S(═O)R^{JA1}, —S(═O)₂R^{JA1}, —OS(═O)₂R^{JA1}, —S(═O)₂OH, —S(═O)₂OR^{JA1};
═O; and
two adjacent groups —R^{S4}, if present, together form —O—CH₂—O— or —O—CH₂CH₂—O—.

In one embodiment, each —R^{S4}, if present, is independently selected from:
—R^{JA1},
—F, —Cl, —Br, —I
—CF₃,
—OH, -L^{JA}-OH,
—OR^{JA1}, -L^{JA}-OR^{JA1},
—CN,
—NH₂, —NHR^{JA1}, —NR^{JA1}₂, —NR^{JA2}R^{JA3},
-L^{JA}-NH₂, -L^{JA}-NHR^{JA1}, -L^{JA}-NR^{JA1}₂,
—C(═O)OH,
—C(═O)R^{JA1},
—C(═O)NH₂, —C(═O)NHR^{JA1}, and —C(═O)NR^{JA1}₂; and
two adjacent groups —R^{S4}, if present, together form —O—CH₂—O— or —O—CH₂CH₂—O—.

Optional Substituents on —Y^{16A}—, —Y^{16B}—, —Y^{17A}—, —Y^{17B}— and -L-

In one embodiment, —Y^{16A}, if present, is unsubstituted.

In one embodiment, —Y^{16A}—, if present, is optionally substituted with one or more substituents, —R^{S5}.

In one embodiment, —Y^{16B}—, if present, is unsubstituted.

In one embodiment, —Y^{16B}—, if present, is optionally substituted with one or more substituents, —R^{S5}.

In one embodiment, —Y^{17A}—, if present, is unsubstituted.

In one embodiment, —Y^{17A}—, if present, is optionally substituted with one or more substituents, —R^{S5}.

In one embodiment, —Y^{17B}—, if present, is unsubstituted.

In one embodiment, —Y^{17B}—, if present, is optionally substituted with one or more substituents, —R^{S5}.

In one embodiment, -L-, if present, is unsubstituted.

In one embodiment, -L-, if present, is optionally substituted with one or more substituents, —R^{S5}.

In one embodiment, each —R^{S5}, if present, is independently selected from:
—R^{JA1},
—F, —Cl, —Br, —I,
—CF₃, —OCF₃, —SCF₃,
—OH, -L^{JA}-OH, —O-L^{JA}-OH, —NH-L^{JA}-OH, —NR^{JA1}-L^{JA}-OH,
—OR^{JA1}, -L^{JA}-OR^{JA1}, —O-L^{JA}-OR^{JA1}, —NH-L^{JA}-OR^{JA1}, —NR^{JA1}-L^{JA}-OR^{JA1},
—SH, —SR^{JA1},
—CN,
—NO₂,
—NH₂, —NHR^{JA1}, —NR^{JA1}₂, —NR^{JA2}R^{JA3},
-L^{JA}-NH₂, -L^{JA}-NHR^{JA1}, -L^{JA}-NR^{JA1}₂, -L^{JA}-NR^{JA2}R^{JA3},
—O-L^{JA}-NH₂, —O-L^{JA}-NHR^{JA1}, —O-L^{JA}-NR^{JA1}₂, —O-L^{JA}-NR^{JA2}R^{JA3},
—NH-L^{JA}-NH₂, —NR^{JA1}-L^{JA}-NH₂, —NH-L^{JA}-NHR^{JA1}, —NR^{JA1}-L^{JA}-NHR^{JA1},
—NH-L^{JA}-NR^{JA1}₂, —NR^{JA1}-L^{JA}-NR^{JA1}₂,
—NH-L^{JA}-NR^{JA2}R^{JA3}, —NR^{JA1}-L^{JA}-NR^{JA2}R^{JA3},
—OC(═O)R^{JA1},
—C(═O)OH, —C(═O)OR^{JA1},
—C(═O)R^{JA1},
—C(═O)NH₂, —C(═O)NHR^{JA1}, —C(═O)NR^{JA1}₂, —C(═O)NR^{JA2}R^{JA3},
—NHC(═O)R^{JA1}, —NR^{JA1}C(═O)R^{JA1},
—NHC(═O)OR^{JA1}, —NR^{JA1}C(═O)OR^{JA1},
—OC(═O)NH₂, —OC(═O)NHR^{JA1}, —OC(═O)NR^{JA1}₂, —OC(═O)NR^{JA2}R^{JA3},
—NHC(═O)NH₂, —NHC(═O)NHR^{JA1},
—NHC(═O)NR^{JA1}₂, —NHC(═O)NR^{JA2}R^{JA3},
—NR^{JA1}C(═O)NH₂, —NR^{JA1}C(═O)NHR^{JA1},
—NR^{JA1}C(═O)NR^{JA1}₂, —NR^{JA1}C(═O)NR^{JA2}R^{JA3},
—NHS(═O)₂R^{JA1}, —NR^{JA1}S(═O)₂R^{JA1},
—S(═O)₂NH₂, —S(═O)₂NHR^{JA1}, —S(═O)₂NR^{JA1}₂, —S(═O)₂NR^{JA2}R^{JA3},
—S(═O)R^{JA1}, —S(═O)₂R^{JA1}, —OS(═O)₂R^{JA1}, —S(═O)₂OH, and —S(═O)₂OR^{JA1}, and
═O.

In one embodiment, each —R^{S5}, if present, is independently selected from:
—R^{JA1},
—F, —Cl, —Br, —I
—CF₃, —OCF₃,
—OH, -L^{JA}-OH,
—OR^{JA1}, -L^{JA}-OR^{JA1},
—SR^{JA1},
—CN,
—NO₂,
—NH₂, —NHR^{JA1}—NR^{JA1}₂, —NR^{JA2}R^{JA3},
—O-L^{JA}-NH₂, —O-L^{JA}-NHR^{JA1}, —O-L^{JA}-NR^{JA1}₂, —O-L^{JA}NR^{JA2}R^{JA3},
—OC(═O)R^{JA1},
—C(═O)OH, —C(═O)OR^{JA1},
—C(═O)R^{JA1}, and
—S(═O)₂R^{JA1}.

In one embodiment, each —R^{S5}, if present, is independently selected from:
—R^{JA1},
—F, —Cl, —Br, —I
—CF₃, —OCF₃,
—OH, -L^{JA}-OH,
—OR^{JA1},
—CN, and
—NH₂, —NHR^{JA1}, —NR^{JA1}₂,

Elements of the Optional Substituents, —R^{S1}, —R^{S2}, —R^{S3}, —R^{S4} and —R^{S5}

Optionally:
each -L^{JA}-, if present, is independently saturated aliphatic C₁₋₅alkylene;
each n is independently 1 to 4;
each —NR^{JA2}R^{JA3}, if present, is independently C₃₋₁₀heterocyclyl, for example independently piperidino, piperazino, morpholino, oxazepino (e.g. homomorpholino) or diazepino (e.g. homopiperazino), and is optionally substituted, for example, with one or more groups selected from —R^{JJ}, —CF₃, —F, —OH, —OR^{JJ}, —NH₂, —NHR^{JJ}, —NR^{JJ}₂, and ═O; wherein each is independently saturated aliphatic C₁₋₄ alkyl;
each —R^{JA1} is independently:
   —R^{JB1}, —R^{JB2}, —R^{JB3}, —R^{JB4}, —R^{JB5}, —R^{JB6}, —R^{JB7}, —R^{JB8},
   -L^{JB}-R^{JB4}, -L^{JB}-R^{JB5}, -L^{JB}-R^{JB6}, -L^{JB}-R^{JB7}, or -L^{JB}-R^{JB8};
each —R^{JB1} is independently saturated aliphatic C₁₋₆alkyl;
each —R^{JB2} is independently aliphatic C₂₋₆alkenyl;
each —R^{JB3} is independently aliphatic C₂₋₆alkynyl;
each —R^{JB4} is independently saturated C₃₋₆cycloalkyl;
each —R^{JB5} is independently C₃₋₆cycloalkenyl;
each —R^{JB6} is independently alicyclic C₄₋₇heterocyclyl;
each —R^{JB7} is independently C₆₋₁₀carboaryl;
each —R^{JB8} is independently C₅₋₁₀heteroaryl;
each -L^{JB}- is independently saturated aliphatic C₁₋₃alkylene;
   wherein:
each —R^{JB4}, —R^{JB5}, —R^{JB6}, —R^{JB7}, and —R^{JB8} is optionally substituted, for example, with one or more substituents —R^{JC1} and/or one or more substituents —R^{JC2},
each —R^{JB1}, —R^{JB2}, —R^{JB3}, and -L^{JB}- is optionally substituted, for example, with one or more substituents —R^{JC2}, and
   wherein:
each —R^{JC1} is independently saturated aliphatic C₁₋₄ alkyl, phenyl, or benzyl;
each —R^{JC2} is independently:
   —F, —Cl, —Br, —I,
   —CF₃, —OCF₃, —SCF₃,
   —OH, -L^{JD}-OH, —O-L^{JD}-OH,
   —OR^{JD1}, -L^{JD}-OR^{JD1}, —O-L^{JD}-OR^{JD1},
   —SH, —SR^{JD1},
   —CN,
   —NO₂,
   —NH₂, —NHR^{JD1}, —NR^{JD1}₂,
   -L^{JD}-NH₂, -L^{JD}-NHR^{JD1}, -L^{JD}-NR^{JD1}₂,
   —C(═O)OH, —C(═O)OR^{JD1},
   —C(═O)NH₂, —C(═O)NHR^{JD1}, or —C(═O)NR^{JD1}₂;
      wherein:
each —R^{JD1} is independently saturated aliphatic C₁₋₄ alkyl, phenyl, or benzyl; and
each -L^{JD}- is independently saturated aliphatic C₁₋₆alkylene;

Optionally, each -L^{JA}-, if present, is independently —(CH₂)ₙ₂—, wherein n2 is independently 1 to 4.

Optionally, each -L^{JA}-, if present, is independently —CH₂— or —CH₂CH₂—.

Optionally, each —NR^{JA2}R^{JA3}, if present, is independently piperidino, piperazino, morpholino, oxazepino (e.g. homomorpholino) or diazepino (e.g. homopiperazino), and is optionally substituted, for example, with one or more groups selected from —R^{JJ}, —CF₃, —F, —OH, —OR^{JJ}, —NH₂, —NHR^{JJ}, —NR^{JJ}₂, and ═O; wherein each —R^{JJ} is independently saturated aliphatic C₁₋₄ alkyl.

Optionally, each —NR^{JA2}R^{JA3}, if present, is independently piperidino, piperazino, morpholino, oxazepino (e.g. homomorpholino) or diazepino (e.g. homopiperazino), and is optionally substituted, for example, with one or more groups selected from —R^{JJ}; wherein each —R^{JJ} is independently saturated aliphatic C₁₋₄ alkyl.

Optionally, each —R^{JA1}, if present, is independently:
—R^{JB1}, —R^{JB4}, —R^{JB6}, —R^{JB7}, —R^{JB8},
-L^{JB}-R^{JB4}, -L^{JB}-R^{JB6}, -L^{JB}R^{JB7}, or -L^{JB}R^{JB8}.

Optionally, each —R^{JA1}, if present, is independently:
—R^{JB1}, —R^{JB7}, —R^{JB8},
-L^{JB}-R^{JB7}, or -L^{JB}-R^{JB8}.

Optionally, each —R^{JA1}, if present, is independently:
—R^{JB1}, —R^{JB7}, or -L^{JB}-R^{JB7}.

Optionally, each —R^{JB7}, if present, is independently phenyl, and is optionally substituted.

Optionally, each —R^{JB8}, if present, is independently C₅₋₆heteroaryl, and is optionally substituted.

Optionally, each —R^{JB8}, if present, is independently C₉₋₁₀heteroaryl, and is optionally substituted.

Optionally, each -L^{JB}-, if present, is independently —CH₂— or —CH₂CH₂—.

Optionally, each -L^{JB}-, if present, is independently —CH₂—.

Optionally, each —R^{JC1}, if present, is independently saturated aliphatic C₁₋₄ alkyl.

Optionally, each —R^{JC2} is independently:
—F, —Cl, —Br, —I,
—OH,
—OR^{JD1},
—CN,
—NO₂,
—NH₂, —NHR^{JD1}, or —NR^{JD1}₂.

Optionally, each —R^{JD1}, if present, is independently saturated aliphatic C₁₋₄ alkyl.

Optionally, each -L^{JD}-, if present, is independently —(CH₂)ₘ₂—, wherein m2 is independently 1 to 4.

Optionally, each -L^{JD}-, if present, is independently —CH₂— or —CH₂CH₂—

### EMBODIMENTS

In embodiments, —R^{17A} is —Y^{17A}Z^{17A}, —Y^{17A} is —CH₂, —CH(CH₃)— or —C(O)— and Z^{17A} is independently phenyl, and is optionally substituted.

In embodiments, —R^{17A} is —Y^{17A}Z^{17A}, —Y^{17A} is —CH₂— and Z^{17A} is independently phenyl, and is optionally substituted.

In embodiments, —Z^{17A} is independently phenyl, and is optionally substituted.

In embodiments, —Z^{17A} is independently 4-substituted phenyl, and is optionally further substituted.

In embodiments, —Z^{17A} is independently 4-substituted phenyl wherein the 4-substituent is 4-chloro, 4-fluoro, 4-bromo, 4-methyl, 4-^{t}butyl, 4-methoxy, 4-CF₃, 4-amino, 4-piperzino, 4-morpholino, 4-piperidino, 4-diazepino (preferably homopiperazino) or 4-oxazepino (preferably homomorpholino).

In embodiments, —Z^{17A} is independently 4-substituted phenyl wherein the 4-substituent is 4-chloro, 4-fluoro, 4-bromo, 4-methoxy, or 4-amino.

In embodiments, —Z^{17A} is independently 4-substituted phenyl wherein the 4-substituent is 4-chloro, 4-fluoro, 4-bromo or 4-methoxy.

In embodiments, —Z^{17A} is independently 4-substituted phenyl wherein the 4-substituent is 4-chloro, 4-fluoro or 4-bromo.

In embodiments, —Z^{17A} is independently 4-substituted phenyl wherein the 4-substituent is 4-chloro or 4-fluoro.

In embodiments, —Z^{17A} is independently 4-chloro substituted phenyl.

In embodiments, —Z^{17A} is independently 2,4-substituted or 3,4-substituted phenyl.

In embodiments, —Z^{17A} is independently 2,4-substituted phenyl.

In embodiments, —R^{17A} is independently selected from the group consisting of:

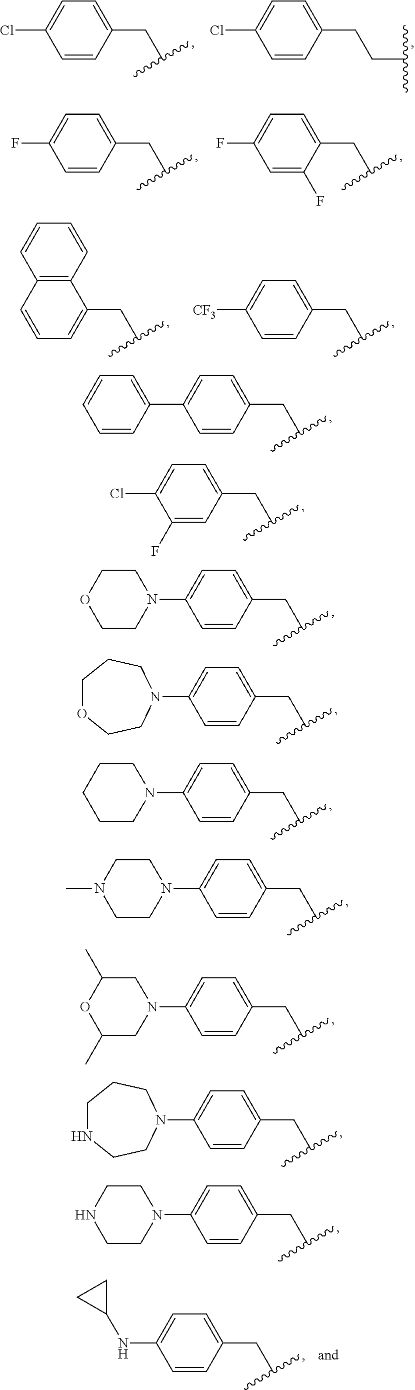

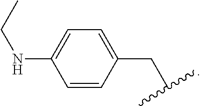

In embodiments, —X¹⁶ is independently —OH.

### Further Embodiments

Further embodiments of the present invention are as follows:
1. A compound selected from compounds of the following formula, and pharmaceutically acceptable salts, hydrates, and solvates thereof:

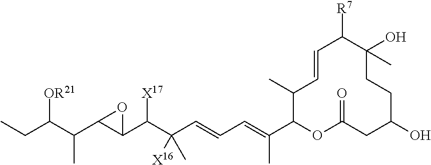

wherein:
—R⁷ is independently —OH, —OR^{7A}, or —O—C(O)R^{7A}
   wherein:
   —R^{7A} is independently —R^{7A1}, —R^{7A2} or —R^{7A3}
      wherein:
      —R^{7A1} is independently saturated or unsaturated aliphatic C₁₋₆alkyl, and is optionally substituted
      and wherein:
      —R^{7A2} is independently saturated or unsaturated alicyclic or aromatic C₃₋₂₀carbocyclyl, and is optionally substituted
      and wherein:
      —R^{7A3} is independently —NH₂, —NHR^{7NA}, —N(R^{7NA})₂, or —NR^{7NB}R^{7NC}
         wherein:
         each —R^{7NA} is independently —R^{7NA1} or —R^{7NA2}
         wherein:
         —R^{7NA1} is independently saturated or unsaturated aliphatic C₁₋₆alkyl, and is optionally substituted
         and wherein:
         —R^{7NA2} is independently saturated or unsaturated alicyclic or aromatic, carbo or hetero, C₃₋₂₀cyclyl, and is optionally substituted
      and wherein:
      —NR^{7NB}R^{7NC} is independently saturated or unsaturated alicyclic or aromatic C₃₋₂₀heterocyclyl, and is optionally substituted
         and wherein:
—X¹⁶ is independently —OR^{16A} or —NR^{16A}R^{16B}
   wherein:
   R^{16A} is independently —H, —Z^{16A}, —Y^{16A}Z^{16A} or together with —R^{17A} is —-L-
      wherein:
      —Y^{16A}— is independently saturated or unsaturated aliphatic C₁₋₄ alkylene, and is optionally substituted
      and wherein:
      -L- is independently saturated or unsaturated aliphatic C₁₋₄ alkylene, —S(O)(O)— or —C(O)—, and is optionally substituted
      and wherein:
      —Z^{16A} is independently —Z^{16A1} or —Z^{16A2}
         wherein:
         —Z^{16A1} is independently saturated or unsaturated aliphatic C₁₋₆alkyl, and is optionally substituted
         and wherein:
         —Z^{16A2} is independently saturated or unsaturated alicyclic or aromatic C₃₋₂₀cyclyl, and is optionally substituted
   and wherein:
   R^{16B} is independently —H, —Z^{16B} or —Y^{16B}Z^{16B}
      wherein:
      —Y^{16B}— is independently saturated or unsaturated aliphatic C₁₋₄ alkylene, and is optionally substituted
      and wherein:
      —Z^{16B} is independently —Z^{16B1} or —Z^{16B2}
         wherein:
         —Z^{16B1} is independently saturated or unsaturated aliphatic C₁₋₆alkyl, and is optionally substituted
         and wherein:
         —Z^{16B2} is independently saturated or unsaturated alicyclic or aromatic C₃₋₂₀cyclyl, and is optionally substituted
            and wherein:
—X¹⁷ is independently —NR^{17A}R^{17B}
wherein:
   —R^{17A} is independently —H, —Y^{17A}Z^{17A} or together with —R^{16A} is -L-
      wherein:
      —Y^{17A}— is independently saturated or unsaturated aliphatic C₁₋₄ alkylene, —C(O)—, or —S(O)(O)—, and is optionally substituted
      and wherein:
      -L- is independently as defined above
      and wherein:
      —Z^{17A} is independently —Z^{17A1} or —Z^{17A2}
         wherein:
         —Z^{17A1} is independently saturated or unsaturated aliphatic C₁₋₆alkyl, and is optionally substituted
         and wherein:
         —Z^{17A2}, if present, is independently —Z^{17AH} or —Z^{17AC}
         wherein:
         —Z^{17AH} is saturated or unsaturated alicyclic or aromatic C₃₋₂₀heterocyclyl, and is optionally substituted
         and wherein:
         —Z^{17AC} is saturated or unsaturated alicyclic or aromatic C₃₋₂₀carbocyclyl, and is optionally substituted
and wherein:
   —R^{17B} is independently —H, —Z^{17B} or —Y^{17B}Z^{17B},
      wherein:
      —Y^{17B}— is independently saturated or unsaturated aliphatic C₁₋₄alkylene, —C(O)—, or —S(O)(O)—, and is optionally substituted
      and wherein:
      —Z^{17B} is independently —Z^{17B1}, —Z^{17B2} or —Z^{17B3}.
         wherein:
         —Z^{17B1} is independently saturated or unsaturated aliphatic C₁₋₆alkyl, and is optionally substituted
         and wherein:
         —Z^{17B2} is independently saturated or unsaturated alicyclic or aromatic C₃₋₂₀carbocyclyl, and is optionally substituted
         and wherein:
         —Z^{17B3} is independently saturated or unsaturated alicyclic or aromatic C₃₋₂₀heterocyclyl, and is optionally substituted
            and wherein:
—R²¹ is independently —H or -Me.
2. A compound according to paragraph 1, wherein —R⁷ is —O—C(O)R^{7A} or —OH.
3. A compound according to paragraph 1, wherein —R⁷ is —OH.
4. A compound according to paragraph 1, wherein —R⁷ is —OR^{7A}.
5. A compound according to paragraph 1, wherein —R⁷ is —O—C(O)R^{7A}.
6. A compound according to any one of paragraphs 1 to 5, wherein —R^{7A}, if present, is independently —R^{7A1}.
7. A compound according to any one of paragraphs 1 to 5, wherein —R^{7A}, if present, is independently —R^{7A2}.
8. A compound according to any one of paragraphs 1 to 5, wherein —R^{7A}, if present, is independently —R^{7A3}.
9. A compound according to any one of paragraphs 1 to 8, wherein —R^{7A1}, if present, is independently saturated or unsaturated aliphatic C₁₋₃alkyl, and is optionally substituted.
10. A compound according to any one of paragraphs 1 to 8, wherein —R^{7A1}, if present, is independently saturated or unsaturated aliphatic C₁₋₂alkyl, and is optionally substituted.
11. A compound according to any one of paragraphs 1 to 8, wherein —R^{7A1}, if present, is independently -Me.
12. A compound according to any one of paragraphs 1 to 11, wherein —R^{7A2}, if present, is independently saturated or unsaturated alicyclic or aromatic C₃₋₁₅carbocyclyl, and is optionally substituted.
13. A compound according to any one of paragraphs 1 to 11, wherein —R^{7A2}, if present, is independently saturated or unsaturated alicyclic or aromatic C₄₋₁₀carbocyclyl, and is optionally substituted.
14. A compound according to any one of paragraphs 1 to 11, wherein —R^{7A2}, if present, is independently saturated or unsaturated alicyclic or aromatic C₅₋₇carbocyclyl, and is optionally substituted.
15. A compound according to any one of paragraphs 1 to 11, wherein —R^{7A2}, if present, is independently saturated or unsaturated alicyclic C₅₋₇carbocyclyl or C₅₋₇carboaryl, and is optionally substituted.
16. A compound according to any one of paragraphs 1 to 15, wherein —R^{7A3}, if present, is independently —NH₂.
17. A compound according to any one of paragraphs 1 to 15, wherein —R^{7A3}, if present, is independently —NHR^{7NA}.
18. A compound according to any one of paragraphs 1 to 15, wherein —R^{7A3}, if present, is independently —N(R^{7NA})₂.
19. A compound according to any one of paragraphs 1 to 15, wherein —R^{7A3}, if present, is independently —NR^{7NB}R^{7NC}.
20. A compound according to any one of paragraphs 1 to 19, wherein —R^{7NA}, if present, is independently —R^{7NA1}.
21. A compound according to any one of paragraphs 1 to 19, wherein —R^{7NA}, if present, is independently —R^{7NA2}.
22. A compound according to any one of paragraphs 1 to 21, wherein —R^{7NA1}, if present, is independently saturated or unsaturated aliphatic C₁₋₃alkyl and is optionally substituted.
23. A compound according to any one of paragraphs 1 to 21, wherein —R^{7NA1}, if present, is independently saturated or unsaturated aliphatic C₁₋₂alkyl and is optionally substituted.
24. A compound according to any one of paragraphs 1 to 21, wherein —R^{7NA1}, if present, is independently -Me.
25. A compound according to any one of paragraphs 1 to 24, wherein —R^{7NA2}, if present, is independently saturated or unsaturated alicyclic or aromatic, carbo or hetero, C₃₋₁₅cyclyl, and is optionally substituted.
26. A compound according to any one of paragraphs 1 to 24, wherein —R^{7NA2}, if present, is independently saturated or unsaturated alicyclic or aromatic, carbo or hetero, C₄₋₁₀cyclyl, and is optionally substituted.
27. A compound according to any one of paragraphs 1 to 24, wherein —R^{7NA2}, if present, is independently saturated or unsaturated alicyclic or aromatic, carbo or hetero, C₅₋₇cyclyl, and is optionally substituted.
28. A compound according to any one of paragraphs 1 to 24, wherein —R^{7NA2}, if present, is independently saturated or unsaturated alicyclic, carbo or hetero, C₅₋₇cyclyl or C₅₋₇aryl, and is optionally substituted.
29. A compound according to any one of paragraphs 1 to 28, wherein —NR^{7NB}R^{7NC}, if present, is independently saturated or unsaturated alicyclic or aromatic C₃₋₁₅heterocyclyl, and is optionally substituted.
30. A compound according to any one of paragraphs 1 to 28, wherein —NR^{7NB}R^{7NC}, if present, is independently saturated or unsaturated alicyclic or aromatic C₄₋₁₀heterocyclyl, and is optionally substituted.
31. A compound according to any one of paragraphs 1 to 28, wherein —NR^{7NB}R^{7NC}, if present, is independently saturated or unsaturated alicyclic or aromatic C₅₋₇heterocyclyl, and is optionally substituted.
32. A compound according to any one of paragraphs 1 to 28, wherein —NR^{7NB}R^{7NC}, if present, is independently saturated or unsaturated alicyclic C₅₋₇heterocyclyl or C₅₋₇ heteroaryl, and is optionally substituted.
33. A compound according to any one of paragraphs 1 to 28, wherein —NR^{7NB}R^{7NC}, if present, is independently azetidino, diazetidino, pyrrolo, pyrrolino, pyrrolidino, imidazolo, imidazolidino, pyrazolo, pyrazolidino, triazolo, pyridino, piperidino, morpholino, pyridazino, piperazino, pyrimidino, pyrazino, azepino or diazepino and is optionally substituted.
34. A compound according to any one of paragraphs 1 to 28, wherein —NR^{7NB}R^{7NC}, if present, is independently piperazino, and is optionally substituted.
35. A compound according to any one of paragraphs 1 to 28, wherein —NR^{7NB}R^{7NC}, if present, is independently:

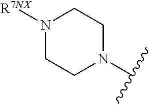

wherein —R^{7NX}, if present, is independently —R^{7NX1} or —R^{7NX2}, wherein:
—R^{7NX1} is independently saturated or unsaturated aliphatic C₁₋₆alkyl, and is optionally substituted; and
—R^{7NX2} is independently saturated or unsaturated alicyclic or aromatic, carbo or hetero, C₃₋₂₀cyclyl and is optionally substituted.
36. A compound according to any one of paragraphs 1 to 35, wherein —R^{7NX}, if present, is independently —R^{7NX1}.
37. A compound according to any one of paragraphs 1 to 35, wherein —R^{7NX}, if present, is independently —R^{7NX2}.
38. A compound according to any one of paragraphs 1 to 37, wherein —R^{7NX1}, if present, is independently saturated or unsaturated aliphatic C₁₋₄ alkyl, and is optionally substituted.
39. A compound according to any one of paragraphs 1 to 37, wherein R^{7NX1}, if present, is independently saturated aliphatic C₁₋₄ alkyl.
40. A compound according to any one of paragraphs 1 to 39, wherein —R^{7NX2}, if present, is independently saturated or unsaturated alicyclic or aromatic, carbo or hetero, C₃₋₁₅cyclyl and is optionally substituted.
41. A compound according to any one of paragraphs 1 to 39, wherein —R^{7NX2}, if present, is independently saturated or unsaturated alicyclic or aromatic, carbo or hetero, C₄₋₁₀cyclyl and is optionally substituted.
42. A compound according to any one of paragraphs 1 to 39, wherein —R^{7NX2}, if present, is independently saturated or unsaturated alicyclic or aromatic, carbo or hetero, C₅₋₇cyclyl and is optionally substituted.
43. A compound according to any one of paragraphs 1 to 39, wherein —R^{7NX2}, if present, is independently or saturated or unsaturated alicyclic or aromatic C₃₋₂₀carbocyclyl and is optionally substituted.
44. A compound according to any one of paragraphs 1 to 39, wherein —R^{7NX2}, if present, is independently saturated or unsaturated alicyclic or aromatic C₃₋₁₅carbocyclyl and is optionally substituted.
45. A compound according to any one of paragraphs 1 to 39, wherein —R^{7NX2}, if present, is independently saturated or unsaturated alicyclic or aromatic C₄₋₁₀carbocyclyl and is optionally substituted.
46. A compound according to any one of paragraphs 1 to 39, wherein —R^{7NX2}, if present, is independently saturated or unsaturated alicyclic or aromatic C₅₋₇carbocyclyl and is optionally substituted.
47. A compound according to any one of paragraphs 1 to 39, wherein —R^{7NX2}, if present, is independently cycloheptyl, and is optionally substituted.
48. A compound according to any one of paragraphs 1 to 47, wherein —X¹⁶ is independently —OR^{16A}.
49. A compound according to any one of paragraphs 1 to 47, wherein —X¹⁶ is independently —NR^{16A}R^{16B}.
50. A compound according to any one of paragraphs 1 to 49, wherein R^{16A} is independently —H.
51. A compound according to any one of paragraphs 1 to 49, wherein R^{16A} is independently —Z^{16A}.
52. A compound according to any one of paragraphs 1 to 49, wherein R^{16A} is independently —Y^{16A}Z^{16A}.
53. A compound according to any one of paragraphs 1 to 49, wherein R^{16A} independently together with —R^{17A} is -L-.
54. A compound according to any one of paragraphs 1 to 53, wherein —Y^{16A}—, if present, is independently saturated or unsaturated aliphatic C₁₋₃alkylene, and is optionally substituted.
55. A compound according to any one of paragraphs 1 to 53, wherein —Y^{16A}—, if present, is independently saturated or unsaturated C₁₋₂alkylene, and is optionally substituted.
56. A compound according to any one of paragraphs 1 to 53, wherein —Y^{16A}—, if present, is independently C₁alkylene, and is optionally substituted.
57. A compound according to any one of paragraphs 1 to 53, wherein —Y^{16A}—, if present, is independently —CH₂—.
58. A compound according to any one of paragraphs 1 to 57, wherein -L-, if present, is independently saturated or unsaturated aliphatic C₁₋₄ alkylene, and is optionally substituted.
59. A compound according to any one of paragraphs 1 to 57, wherein -L-, if present, is independently —S(O)(O)—.
60. A compound according to any one of paragraphs 1 to 57, wherein -L-, if present, is independently —C(O)—.
61. A compound according to any one of paragraphs 1 to 57, wherein -L-, if present, is independently saturated or unsaturated aliphatic C₁₋₃alkylene, and is optionally substituted.
62. A compound according to any one of paragraphs 1 to 57, wherein -L-, if present, is independently saturated or unsaturated aliphatic C₁₋₂alkylene, and is optionally substituted.
63. A compound according to any one of paragraphs 1 to 57, wherein -L-, if present, is independently C₂alkylene, and is optionally substituted.
64. A compound according to any one of paragraphs 1 to 57, wherein -L-, if present, is independently —CH₂—CH₂— or —CH(Ph)—CH(Ph)—.
65. A compound according to any one of paragraphs 1 to 57, wherein -L-, if present, is independently —CH₂—CH₂—.
66. A compound according to any one of paragraphs 1 to 57, wherein -L-, if present, is independently —CH(Ph)—CH(Ph)—.
67. A compound according to any one of paragraphs 1 to 66, wherein —Z^{16A}, if present, is independently —Z^{16A1}.
68. A compound according to any one of paragraphs 1 to 66, wherein —Z^{16A}, if present, is independently —Z^{16A2}.
69. A compound according to any one of paragraphs 1 to 68, wherein —Z^{16A1}, if present, is independently saturated or unsaturated aliphatic C₁₋₃alkyl, and is optionally substituted.
70. A compound according to any one of paragraphs 1 to 68, wherein —Z^{16A1}, if present, is independently saturated or unsaturated C₁₋₂alkyl, and is optionally substituted.
71. A compound according to any one of paragraphs 1 to 68, wherein —Z^{16A1}, if present, is independently C₁ alkyl, and is optionally substituted.
72. A compound according to any one of paragraphs 1 to 68, wherein —Z^{16A1}, if present, is independently -Me.
73. A compound according to any one of paragraphs 1 to 72, wherein —Z^{16A2}, if present, is independently saturated or unsaturated alicyclic or aromatic C₃₋₁₅cyclyl, and is optionally substituted.
74. A compound according to any one of paragraphs 1 to 72, wherein —Z^{16A2}, if present, is independently saturated or unsaturated alicyclic or aromatic C₄₋₁₀cyclyl, and is optionally substituted.
75. A compound according to any one of paragraphs 1 to 72, wherein —Z^{16A2}, if present, is independently saturated or unsaturated alicyclic or aromatic C₅₋₇cyclyl, and is optionally substituted.
76. A compound according to any one of paragraphs 1 to 72, wherein —Z^{16A2}, if present, is independently saturated or unsaturated alicyclic or aromatic C₆cyclyl, and is optionally substituted.
77. A compound according to any one of paragraphs 1 to 72, wherein —Z^{16A2}, if present, is independently saturated or unsaturated alicyclic or aromatic C₃₋₂₀carbocyclyl, and is optionally substituted.
78. A compound according to any one of paragraphs 1 to 72, wherein —Z^{16A2}, if present, is independently saturated or unsaturated alicyclic or aromatic C₃₋₁₅carbocyclyl, and is optionally substituted.
79. A compound according to any one of paragraphs 1 to 72, wherein —Z^{16A2}, if present, is independently saturated or unsaturated alicyclic or aromatic C₄₋₁₀carbocyclyl, and is optionally substituted.
80. A compound according to any one of paragraphs 1 to 72, wherein —Z^{16A2}, if present, is independently saturated or unsaturated alicyclic or aromatic C₅₋₇carbocyclyl, and is optionally substituted.
81. A compound according to any one of paragraphs 1 to 72, wherein —Z^{16A2}, if present, is independently saturated or unsaturated alicyclic or aromatic C₆carbocyclyl, and is optionally substituted.
82. A compound according to any one of paragraphs 1 to 72, wherein —Z^{16A2}, if present, is independently saturated or unsaturated alicyclic C₅₋₇carbocyclyl or C₅₋₇carboaryl, and is optionally substituted.
83. A compound according to any one of paragraphs 1 to 72, wherein —Z^{16A2}, if present, is independently phenyl, and is optionally substituted.
84. A compound according to any one of paragraphs 1 to 83, wherein —R^{16B}, if present, is independently —H.
85. A compound according to any one of paragraphs 1 to 83, wherein —R^{16B}, if present, is independently —Z^{16B}.
86. A compound according to any one of paragraphs 1 to 83, wherein —R^{16B}, if present, is independently —Y^{16B}Z^{16B}.
87. A compound according to any one of paragraphs 1 to 86, wherein —Y^{16B}—, if present, is independently saturated or unsaturated aliphatic C₁₋₃alkylene, and is optionally substituted.
88. A compound according to any one of paragraphs 1 to 86, wherein —Y^{16B}—, if present, is independently saturated or unsaturated C₁₋₂alkylene, and is optionally substituted.
89. A compound according to any one of paragraphs 1 to 86, wherein —Y^{16B}—, if present, is independently C₁alkylene, and is optionally substituted.
90. A compound according to any one of paragraphs 1 to 86, wherein —Y^{16B}—, if present, is independently —CH₂—.
91. A compound according to any one of paragraphs 1 to 90, wherein —Z^{16B}, if present, is independently —Z^{16B1}.
92. A compound according to any one of paragraphs 1 to 90, wherein —Z^{16B}, if present, is independently —Z¹⁶³².
93. A compound according to any one of paragraphs 1 to 92, wherein —Z^{16B1}, if present, is independently saturated or unsaturated aliphatic C₁₋₃alkyl, and is optionally substituted.
94. A compound according to any one of paragraphs 1 to 92, wherein —Z^{16B1}, if present, is independently saturated or unsaturated C₁₋₂alkyl, and is optionally substituted.
95. A compound according to any one of paragraphs 1 to 92, wherein —Z^{16B1}, if present, is independently C₁alkyl, and is optionally substituted.
96. A compound according to any one of paragraphs 1 to 92, wherein —Z^{16B1}, if present, is independently -Me.
97. A compound according to any one of paragraphs 1 to 96, wherein —Z^{16B2}, if present, is independently saturated or unsaturated alicyclic or aromatic C₃₋₁₅cyclyl, and is optionally substituted.
98. A compound according to any one of paragraphs 1 to 96, wherein —Z^{16B2}, if present, is independently saturated or unsaturated alicyclic or aromatic C₄₋₁₀cyclyl, and is optionally substituted.
99. A compound according to any one of paragraphs 1 to 96, wherein —Z^{16B2}, if present, is independently saturated or unsaturated alicyclic or aromatic C₅₋₇cyclyl, and is optionally substituted.
100. A compound according to any one of paragraphs 1 to 96, wherein —Z^{16B2}, if present, is independently saturated or unsaturated alicyclic or aromatic C₆cyclyl, and is optionally substituted.
101. A compound according to any one of paragraphs 1 to 96, wherein —Z^{16B2}, if present, is independently saturated or unsaturated alicyclic or aromatic C₃₋₂₀carbocyclyl, and is optionally substituted.
102. A compound according to any one of paragraphs 1 to 96, wherein —Z^{16B2}, if present, is independently saturated or unsaturated alicyclic or aromatic C₃₋₁₅carbocyclyl, and is optionally substituted.
103. A compound according to any one of paragraphs 1 to 96, wherein —Z^{16B2}, if present, is independently saturated or unsaturated alicyclic or aromatic C₄₋₁₀carbocyclyl, and is optionally substituted.
104. A compound according to any one of paragraphs 1 to 96, wherein —Z^{16B2}, if present, is independently saturated or unsaturated alicyclic or aromatic C₅₋₇carbocyclyl, and is optionally substituted.
105. A compound according to any one of paragraphs 1 to 96, wherein —Z^{16B2}, if present, is independently saturated or unsaturated alicyclic or aromatic C₆carbocyclyl, and is optionally substituted.
106. A compound according to any one of paragraphs 1 to 96, wherein —Z^{16B2}, if present, is independently saturated or unsaturated alicyclic C₆carbocyclyl or C₅₋₇carboaryl, and is optionally substituted.
107. A compound according to any one of paragraphs 1 to 96, wherein —Z^{16B2}, if present, is independently phenyl, and is optionally substituted.
108. A compound according to any one of paragraphs 1 to 107, wherein R^{17A} is independently —H.
109. A compound according to any one of paragraphs 1 to 107, wherein R^{17A} is independently —Z^{17A}, and is optionally substituted.
110. A compound according to any one of paragraphs 1 to 107, wherein R^{17A} is independently —Y^{17A}Z^{17A}, and is optionally substituted.
111. A compound according to any one of paragraphs 1 to 107, wherein R^{17A} independently together with —R^{16A} is -L-, and is optionally substituted.
112. A compound according to any one of paragraphs 1 to 111, wherein —Y^{17A}—, if present, is independently saturated or unsaturated aliphatic C₁₋₄ alkylene and is optionally substituted.
113. A compound according to any one of paragraphs 1 to 111, wherein —Y^{17A}—, if present, is independently —C(O)—.
114. A compound according to any one of paragraphs 1 to 111, wherein —Y^{17A}—, if present, is independently —S(O)(O)—.
115. A compound according to any one of paragraphs 1 to 111, wherein —Y^{17A}—, if present, is independently saturated or unsaturated aliphatic C₁₋₄alkylene, and is optionally substituted.
116. A compound according to any one of paragraphs 1 to 111, wherein —Y^{17A}—, if present, is independently saturated or unsaturated aliphatic C₁₋₃alkylene, and is optionally substituted.
117. A compound according to any one of paragraphs 1 to 111, wherein —Y^{17A}—, if present, is independently saturated or unsaturated C₁₋₂alkylene, and is optionally substituted.
118. A compound according to any one of paragraphs 1 to 111, wherein —Y^{7A}—, if present, is independently C₁alkylene, and is optionally substituted.
119. A compound according to any one of paragraphs 1 to 111, wherein —Y^{17A}—, if present, is independently —CH₂—, —C(CH₃)H—, —CH₂—CH₂— or —CH₂—CH₂—CH₂—.
120. A compound according to any one of paragraphs 1 to 111, wherein —Y^{17A}—, if present, is independently —CH₂—.
121. A compound according to any one of paragraphs 1 to 111, wherein —Y^{17A}—, if present, is independently —C(CH₃)H—.
122. A compound according to any one of paragraphs 1 to 111, wherein —Y^{17A}—, if present, is independently —CH₂—CH₂—.
123. A compound according to any one of paragraphs 1 to 111, wherein —Y^{17A}—, if present, is independently —CH₂—CH₂—CH₂—.
124. A compound according to any one of paragraphs 1 to 123, wherein —Z^{17A}, if present, is independently —Z^{17A1}.
125. A compound according to any one of paragraphs 1 to 123, wherein —Z^{17A}, if present, is independently —Z^{17A2}.
126. A compound according to any one of paragraphs 1 to 125, wherein —Z^{17A1}, if present, is independently saturated or unsaturated aliphatic C₁₋₄alkyl, and is optionally substituted.
127. A compound according to any one of paragraphs 1 to 125, wherein —Z^{17A1}, if present, is independently saturated or unsaturated aliphatic C₁₋₃alkyl, and is optionally substituted.
128. A compound according to any one of paragraphs 1 to 125, wherein —Z^{17A1}, if present, is independently saturated or unsaturated C₁₋₃alkyl, and is optionally substituted.
129. A compound according to any one of paragraphs 1 to 125, wherein —Z^{17A1}, if present, is independently methyl, ethyl or propyl and is optionally substituted.
130. A compound according to any one of paragraphs 1 to 125, wherein —Z^{17A1}, if present, is independently methyl, and is optionally substituted.
131. A compound according to any one of paragraphs 1 to 125, wherein —Z^{17A1}, if present, is independently ethyl and is optionally substituted.
132. A compound according to any one of paragraphs 1 to 125, wherein —Z^{17A1}, if present, is independently propyl and is optionally substituted.
133. A compound according to any one of paragraphs 1 to 132, wherein —Z^{17AH}, if present, is independently saturated or unsaturated alicyclic or aromatic C₃₋₁₅heterocyclyl, and is optionally substituted.
134. A compound according to any one of paragraphs 1 to 132, wherein —Z^{17AH}, if present, is independently saturated or unsaturated alicyclic or aromatic C₄₋₁₀heterocyclyl, and is optionally substituted.
135. A compound according to any one of paragraphs 1 to 132, wherein —Z^{17AH}, if present, is independently saturated or unsaturated alicyclic or aromatic C₅₋₇heterocyclyl, and is optionally substituted.
136. A compound according to any one of paragraphs 1 to 132, wherein —Z^{17AH}, if present, is independently saturated or unsaturated alicyclic or aromatic C₆heterocyclyl, and is optionally substituted.
137. A compound according to any one of paragraphs 1 to 132, wherein —Z^{17AH}, if present, is independently saturated or unsaturated alicyclic C₅₋₇heterocyclyl or C₅₋₇heteroaryl, and is optionally substituted.
138. A compound according to any one of paragraphs 1 to 132, wherein —Z^{17AH}, if present, is independently piperazino or morpholino, and is optionally substituted.
139. A compound according to any one of paragraphs 1 to 132, wherein —Z^{17AH}, if present, is independently piperazino, and is optionally substituted.
140. A compound according to any one of paragraphs 1 to 132, wherein —Z^{17AH}, if present, is independently morpholino, and is optionally substituted.
141. A compound according to any one of paragraphs 1 to 132, wherein —Z^{17AH}, if present, is independently pyridinyl, indolyl, pyrazinyl, pyrrolidinyl, imidazolyl, pyrazolyl, triazolyl, tetrazolyl, furanyl and thiophenyl, and is optionally substituted.
142. A compound according to any one of paragraphs 1 to 132, wherein —Z^{17AH}, if present, is independently pyridinyl, or indolyl, and is optionally substituted.
143. A compound according to any one of paragraphs 1 to 132, wherein —Z^{17AH}, if present, is independently pyridinyl, and is optionally substituted.
144. A compound according to any one of paragraphs 1 to 132, wherein —Z^{17AH}, if present, is independently pyridine-2-yl, pyridine-3-yl or pyridine-4-yl, and is optionally substituted.
145. A compound according to any one of paragraphs 1 to 132, wherein —Z^{17AH}, if present, is independently indolyl, and is optionally substituted.
146. A compound according to any one of paragraphs 1 to 132, wherein —Z^{17AH}, if present, is independently indol-5-yl, and is optionally substituted.
147. A compound according to any one of paragraphs 1 to 146, wherein —Z^{17AC}, if present, is independently saturated or unsaturated alicyclic or aromatic C₃₋₁₅carbocyclyl, and is optionally substituted.
148. A compound according to any one of paragraphs 1 to 146, wherein —Z^{17AC}, if present, is independently saturated or unsaturated alicyclic or aromatic C₄₋₁₀carbocyclyl, and is optionally substituted.
149. A compound according to any one of paragraphs 1 to 146, wherein —Z^{17AC}, if present, is independently saturated or unsaturated alicyclic or aromatic C₅₋₇carbocyclyl, and is optionally substituted.
150. A compound according to any one of paragraphs 1 to 146, wherein —Z^{17AC}, if present, is independently saturated or unsaturated alicyclic or aromatic C₆carbocyclyl, and is optionally substituted.
151. A compound according to any one of paragraphs 1 to 146, wherein —Z^{17AC}, if present, is independently C₆₋₁₀carboaryl, and is optionally substituted.
152. A compound according to any one of paragraphs 1 to 146, wherein —Z^{17AC}, if present, is independently C₅₋₇carboaryl, and is optionally substituted.
153. A compound according to any one of paragraphs 1 to 146, wherein —Z^{17AC}, if present, is independently C₆carboaryl, and is optionally substituted.
154. A compound according to any one of paragraphs 1 to 146, wherein —Z^{17AC}, if present, is independently saturated or unsaturated alicyclic C₅₋₇carbocyclyl or C₅₋₇carboaryl, and is optionally substituted.
155. A compound according to any one of paragraphs 1 to 146, wherein —Z^{17AC}, if present, is independently cyclopropyl, cyclobutyl, cyclohexyl or cycloheptyl, and is optionally substituted.
156. A compound according to any one of paragraphs 1 to 146, wherein —Z^{17AC}, if present, is independently cyclopropyl, and is optionally substituted.
157. A compound according to any one of paragraphs 1 to 146, wherein —Z^{17AC}, if present, is independently cyclobutyl, and is optionally substituted.
158. A compound according to any one of paragraphs 1 to 146, wherein —Z^{17AC}, if present, is independently cyclohexyl, and is optionally substituted.
159. A compound according to any one of paragraphs 1 to 146, wherein —Z^{17AC}, if present, is independently cycloheptyl, and is optionally substituted.
160. A compound according to any one of paragraphs 1 to 146, wherein —Z^{17AC}, if present, is independently phenyl or naphthyl, and is optionally substituted.
161. A compound according to any one of paragraphs 1 to 146, wherein —Z^{17AC}, if present, is independently phenyl, and is optionally substituted.
162. A compound according to any one of paragraphs 1 to 146, wherein —Z^{17AC}, if present, is independently naphthyl, and is optionally substituted.
163. A compound according to any one of paragraphs 1 to 162, wherein —R^{17B} is independently —H.
164. A compound according to any one of paragraphs 1 to 162, wherein —R^{17B} is independently —Z^{17B}.
165. A compound according to any one of paragraphs 1 to 162, wherein —R^{17B} is independently —Y^{17B}Z^{17B}.
166. A compound according to any one of paragraphs 1 to 165, wherein —Y^{17B}—, if present, is independently saturated or unsaturated aliphatic C₁₋₄ alkylene and is optionally substituted.
167. A compound according to any one of paragraphs 1 to 165, wherein, —Y^{17B}—, if present, is independently —C(O)—.
168. A compound according to any one of paragraphs 1 to 165, wherein —Y^{17B}—, if present, is independently —S(O)(O)—.
169. A compound according to any one of paragraphs 1 to 165, wherein —Y^{17B}—, if present, is independently saturated or unsaturated aliphatic C₁₋₃alkylene, and is optionally substituted.
170. A compound according to any one of paragraphs 1 to 165, wherein —Y^{17B}—, if present, is independently saturated or unsaturated C₁₋₂alkylene, and is optionally substituted.
171. A compound according to any one of paragraphs 1 to 165, wherein —Y^{17B}—, if present, is independently C₁alkylene, and is optionally substituted.
172. A compound according to any one of paragraphs 1 to 165, wherein —Y^{17B}—, if present, is independently —CH₂—, —C(CH₃)H—, —CH₂—CH₂— or —CH₂—CH₂—CH₂—.
173. A compound according to any one of paragraphs 1 to 165, wherein —Y^{17B}—, if present, is independently —CH₂—.
174. A compound according to any one of paragraphs 1 to 165, wherein —Y^{17B}—, if present, is independently —C(CH₃)H—.
175. A compound according to any one of paragraphs 1 to 165, wherein —Y^{17B}—, if present, is independently —CH₂—CH₂—.
176. A compound according to any one of paragraphs 1 to 165, wherein —Y^{17B}—, if present, is independently —CH₂—CH₂—CH₂—.
177. A compound according to any one of paragraphs 1 to 176, wherein —Z^{17B}, if present, is independently —Z^{17B1}.
178. A compound according to any one of paragraphs 1 to 176, wherein —Z^{17B}, if present, is independently —Z^{17B2}.
179. A compound according to any one of paragraphs 1 to 176, wherein —Z^{17B}, if present, is independently —Z^{17B3}.
180. A compound according to any one of paragraphs 1 to 179, wherein —Z^{17B1}, if present, is independently saturated or unsaturated aliphatic C₁₋₄ alkyl, and is optionally substituted.
181. A compound according to any one of paragraphs 1 to 179, wherein —Z^{17B1}, if present, is independently saturated or unsaturated aliphatic C₁₋₃alkyl, and is optionally substituted.
182. A compound according to any one of paragraphs 1 to 179, wherein —Z^{17B1}, if present, is independently saturated aliphatic C₁₋₃alkyl, and is optionally substituted.
183. A compound according to any one of paragraphs 1 to 179, wherein —Z^{17B1}, if present, is independently methyl, ethyl or propyl and is optionally substituted.
184. A compound according to any one of paragraphs 1 to 179, wherein —Z^{17B1}, if present, is independently methyl, and is optionally substituted.
185. A compound according to any one of paragraphs 1 to 179, wherein —Z^{17B1}, if present, is independently ethyl and is optionally substituted.
186. A compound according to any one of paragraphs 1 to 179, wherein —Z^{17B1}, if present, is independently propyl and is optionally substituted.
187. A compound according to any one of paragraphs 1 to 186, wherein —Z^{17B2}, if present, is independently saturated or unsaturated alicyclic or aromatic C₃₋₁₅carbocyclyl, and is optionally substituted.
188. A compound according to any one of paragraphs 1 to 186, wherein —Z¹⁷⁶², if present, is independently saturated or unsaturated alicyclic or aromatic C₄₋₁₀carbocyclyl, and is optionally substituted.
189. A compound according to any one of paragraphs 1 to 186, wherein —Z^{17B2}, if present, is independently saturated or unsaturated alicyclic or aromatic C₅₋₇carbocyclyl, and is optionally substituted.
190. A compound according to any one of paragraphs 1 to 186, wherein —Z^{17B2}, if present, is independently saturated or unsaturated alicyclic or aromatic C₆carbocyclyl, and is optionally substituted.
191. A compound according to any one of paragraphs 1 to 186, wherein —Z^{17B2}, if present, is independently saturated or unsaturated alicyclic C₅₋₇carbocyclyl or C₅₋₇carboaryl, and is optionally substituted.
192. A compound according to any one of paragraphs 1 to 186, wherein —Z^{17B2}, if present, is independently phenyl, and is optionally substituted.
193. A compound according to any one of paragraphs 1 to 192, wherein —Z^{17B3}, if present, is independently saturated or unsaturated alicyclic or aromatic C₃₋₁₅heterocyclyl, and is optionally substituted.
194. A compound according to any one of paragraphs 1 to 192, wherein —Z^{17B3}, if present, is independently saturated or unsaturated alicyclic or aromatic C₄₋₁₀heterocyclyl, and is optionally substituted.
195. A compound according to any one of paragraphs 1 to 192, wherein —Z^{17B3}, if present, is independently saturated or unsaturated alicyclic or aromatic C₅₋₇heterocyclyl, and is optionally substituted.
196. A compound according to any one of paragraphs 1 to 192, wherein —Z^{17B3}, if present, is independently saturated or unsaturated alicyclic or aromatic C₆heterocyclyl, and is optionally substituted.
197. A compound according to any one of paragraphs 1 to 192, wherein —Z¹⁷⁶³, if present, is independently saturated or unsaturated alicyclic C₅₋₇heterocyclyl or C₅₋₇heteroaryl, and is optionally substituted.
198. A compound according to any one of paragraphs 1 to 192, wherein —Z^{17B3}, if present, independently contains at least one nitrogen ring atom.
199. A compound according to any one of paragraphs 1 to 192, wherein —Z^{17B3}, if present, independently contains one nitrogen ring atom.
200. A compound according to any one of paragraphs 1 to 192, wherein —Z^{17B3}, if present, is independently pyridinyl, and is optionally substituted.
201. A compound according to any one of paragraphs 1 to 200, wherein —R²¹ is independently —H or -Me.
202. A compound according to any one of paragraphs 1 to 200, wherein —R²¹ is independently —H.
203. A compound according to any one of paragraphs 1 to 200, wherein —R²¹ is independently -Me.
204. A compound according to any one of paragraphs 1 to 203, wherein:

each of —R^{7A1}, if present, —R^{7NA1}, if present, —Z^{16A1}, if present, —Z^{16B1}, if present, —Z^{17A1}, if present, and —Z^{17B1}, if present, is optionally substituted with one or more substituents, —R^{S1}, wherein each —R^{S1} is independently selected from:
—R^{JA1},
—F, —Cl, —Br, —I,
—CF₃, —OCF₃, —SCF₃,
—OH, -L^{JA}-OH, —O-L^{JA}-OH, —NH-L^{JA}-OH, —NR^{JA1}-L^{JA}-OH,
—OR^{JA1}, -L^{JA}OR^{JA1}, —OR^{JA1}, —NH-L^{JA}-OR^{JA1}, —NR^{JA1}-L^{JA}-OR^{JA1},
—SH, —SR^{JA1},
—CN,
—NO₂,
—NH₂, —NHR^{JA1}, —NR^{JA1}₂, —NR^{JA2}R^{JA3},
-L^{JA}-NH₂, -L^{JA}-NHR^{JA1}, -L^{JA}-NR^{JA1}₂, -L^{JA}-NR^{JA2}R^{JA3},
—O-L^{JA}-NH₂, —O-L^{JA}-NHR^{JA1}, —O-L^{JA}-NR^{JA1}₂, —O-L^{JA}-NR^{JA2}R^{JA3},
—NH-L^{JA}-NH₂, —NR^{JA1}-L^{JA}-NH₂, —NH-L^{JA}-NHR^{JA1}, —NR^{JA1}-L^{JA}-NHR^{JA1},
—NH-L^{JA}-NR^{JA1}₂, —NR^{JA1}-L^{JA}-NR^{JA1}₂,
—NH-L^{JA}-NR^{JA2}R^{JA3}, —NR^{JA1}-L^{JA}-NR^{JA2}R^{JA3},
—OC(═O)R^{JA1},
—C(═O)OH, —C(═O)OR^{JA1},
—C(═O)R^{JA1},
—C(═O)NH₂, —C(═O)NHR^{JA1}, —C(═O)NR^{JA1}₂, —C(═O)NR^{JA2}R^{JA3},
—NHC(═O)R^{JA1}, —NR^{JA1}C(═O)R^{JA1},
—NHC(═O)OR^{JA1}, —NR^{JA1}C(═O)OR^{JA1},
—OC(═O)NH₂, —OC(═O)NHR^{JA1}, —OC(═O)NR^{JA1}₂, —OC(═O)NR^{JA2}R^{JA3},
—NHC(═O)NH₂, —NHC(═O)NHR^{JA1},
—NHC(═O)NR^{JA1}₂, —NHC(═O)NR^{JA2}R^{JA3},
—NR^{JA1}C(═O)NH₂, —NR^{JA1}C(═O)NHR^{JA1},
—NR^{JA1}C(═O)NR^{JA1}₂, —NR^{JA1}C(═O)NR^{JA2}R^{JA3},
—NHS(═O)₂R^{JA1}, —NR^{JA1}S(═O)₂R^{JA1},
—S(═O)₂NH₂, —S(═O)₂NHR^{JA1}, —S(═O)₂NR^{JA1}₂, —S(═O)₂NR^{JA2}R^{JA3},
—S(═O)R^{JA1}, —S(═O)₂R^{JA1}, —OS(═O)₂R^{JA1}, —S(═O)₂OH, —S(═O)₂OR^{JA1}; and
═O;
   each of —R^{7A2}, if present, —R^{7NA2}, if present, —Z^{16A2}, if present, and —Z^{16B2}, if present, is optionally substituted with one or more substituents, —R^{S2}, wherein each —R^{S2} is independently selected from:
—R^{JA1},
—F, —Cl, —Br, —I,
—CF₃, —OCF₃, —SCF₃,
—OH, -L^{JA}-OH, —O-L^{JA}-OH, —NH-L^{JA}-OH, —NR^{JA1}-L^{JA}-OH,
—OR^{JA1}, -L^{JA}-OR^{JA1}, —O-L^{JA}-OR^{A1}, —NH-L^{JA}-OR^{JA1}, —NR^{JA1}-L^{JA}-OR^{JA1},
—SH, —SR^{JA1},
—CN,
—NO₂,
—NH₂, —NHR^{JA1}, —NR^{JA1}₂, —NR^{JA2}R^{JA3},
-L^{JA}-NH₂, -L^{JA}-NHR^{JA1}, -L^{JA}-NR^{JA1}₂, -L^{JA}-NR^{JA2}R^{JA3},
—O-L^{JA}-NH₂, —O-L^{JA}-NHR^{JA1}, —O-L-NR^{JA1}₂, —O-L^{JA}-NR^{JA2}R^{JA3},
—NH-L^{JA}-NH₂, —NR^{JA1}-L^{JA}-NH₂, —NH-L^{JA}-NHR^{JA1}, —NR^{JA1}-L^{JA}-NHR^{JA1},
—NH-L^{JA}-NR^{JA1}₂, —NR^{JA1}-L^{JA}-NR^{JA1}₂,
—NH-L^{JA}-NR^{JA2}R^{JA3}, —NR^{JA1}-L^{JA}-NR^{JA2}R^{JA3},
—OC(═O)R^{JA1},
—C(═O)OH, —C(═O)OR^{JA1},
—C(═O)R^{JA1},
—C(═O)NH₂, —C(═O)NHR^{JA1}, —C(═O)NR^{JA1}₂, —C(═O)NR^{JA2}R^{JA3},
—NHC(═O)R^{JA1}, —NR^{JA1}C(═O)R^{JA1},
—NHC(═O)OR^{JA1}, —NR^{JA1}C(═O)OR^{JA1},
—OC(═O)NH₂, —OC(═O)NHR^{JA1}, —OC(═O)NR^{JA1}₂, —OC(═O)NR^{JA2}R^{JA3},
—NHC(═O)NH₂, —NHC(═O)NHR^{JA1},
—NHC(═O)NR^{JA1}₂, —NHC(═O)NR^{JA2}R^{JA3},
—NR^{JA1}C(═O)NH₂, —NR^{JA1}C(═O)NHR^{JA1},
—NR^{JA1}C(═O)NR^{JA1}₂, —NR^{JA1}C(═O)NR^{JA2}R^{JA3},
—NHS(═O)₂R^{JA1}, NR^{JA1}S(═O)₂R^{JA1},
—S(═O)₂NH₂, —S(═O)₂NHR^{JA1}, —S(═O)₂NR^{JA1}₂, —S(═O)₂NR^{JA2}R^{JA3},
—S(═O)R^{JA1}, —S(═O)₂R^{JA1}, —OS(═O)₂R^{JA1}, —S(═O)₂OH, —S(═O)₂OR^{JA1},
═O; and
two adjacent groups —R^{S2}, if present, together form —O—CH₂—O— or —O—CH₂CH₂—O—;
   each of —NR^{7NB}R^{7NC}, if present, —Z^{16B3}, if present, and —Z^{17AH}, if present, is optionally substituted with one or more substituents, —R^{S3}, wherein each —R^{S3} is independently selected from:
—R^{JA1},
—F, —Cl, —Br, —I,
—CF₃, —OCF₃, —SCF₃,
—OH, -L^{JA}-OH, —O-L^{JA}-OH, —NH-L^{JA}-OH, —NR^{JA1}-L^{JA}-OH,
—OR^{JA1}, -L^{JA}-OR^{JA1}, —O-L^{JA}-OR^{JA1}, —NH-L^{JA}-OR^{JA1}, —NR^{JA1}-L^{JA}-OR^{JA1},
—SH, —SR^{JA1},
—CN,
—NO₂,
—NH₂, —NHR^{JA1}, —NR^{JA1}₂, —NR^{JA2}R^{JA3},
-L^{JA}-NH₂, -L^{JA}-NHR^{JA1}, -L^{JA}-NR^{JA1}₂, -L^{JA}-NR^{JA2}R^{JA3},
—O-L^{JA}-NH₂, —O-L^{JA}-NHR^{JA1}, —O-L^{JA}-NR^{JA1}₂, —O-L^{JA}NR^{JA2}R^{JA3},
—NH-L^{JA}-NH₂, —NR^{JA1}-L^{JA}-NH₂, —NH-L^{JA}-NHR^{JA1}, —NR^{JA1}-L^{JA}-NHR^{JA1},
—NH-L^{JA}-NR^{JA1}₂, —NR^{JA1}-L^{JA}-NR^{JA1}₂,
—NH-L^{JA}-NR^{JA2}R^{JA3}, —NR^{JA1}-L^{JA}-NR^{JA2}R^{JA3},
—OC(═O)R^{JA1},
—C(═O)OH, —C(═O)OR^{JA1},
—C(═O)R^{JA1},
—C(═O)NH₂, —C(═O)NHR^{JA1}, —C(═O)NR^{JA1}₂, —C(═O)NR^{JA2}R^{JA3},
—NHC(═O)R^{JA1}, —NR^{JA1}C(═O)R^{JA1},
—NHC(═O)OR^{JA1}, —NR^{JA1}C(═O)OR^{JA1},
—OC(═O)NH₂, —OC(═O)NHR^{JA1}, —OC(═O)NR^{JA1}₂, —OC(═O)NR^{JA2}R^{JA3},
—NHC(═O)NH₂, —NHC(═O)NHR^{JA1},
—NHC(═O)NR^{JA1}₂, —NHC(═O)NR^{JA2}R^{JA3},
—NR^{JA1}C(═O)NH₂, —NR^{JA1}C(═O)NHR^{JA1},
—NR^{JA1}C(═O)NR^{JA1}₂, —NR^{JA1}C(═O)NR^{JA2}R^{JA3},
—NHS(═O)₂R^{JA1}, —NR^{JA1}S(═O)₂R^{JA1},
—S(═O)₂NH₂, —S(═O)₂NHR^{JA1}, —S(═O)₂NR^{JA1}₂, —S(═O)₂NR^{JA2}R^{JA3},
—S(═O)R^{JA1}, —S(═O)₂R^{JA1}, —OS(═O)₂R^{JA1}, —S(═O)₂OH, —S(═O)₂OR^{JA1},
═O; and
two adjacent groups —R^{S3}, if present, together form —O—CH₂—O— or —O—CH₂CH₂—O—;
   each of —Z^{17AC}, if present, —Z^{17B2}, if present, and —Z^{17B3}, if present, is optionally substituted with one or more substituents, —R^{S4}, wherein each —R^{S4} is independently selected from:
—R^{JA1},
—F, —Cl, —Br, —I,
—CF₃, —OCF₃, —SCF₃,
—OH, -L^{JA}-OH, —O-L^{JA}-OH, —NH-L^{JA}-OH, —NR^{JA1}-L^{JA}-OH,
—OR^{JA1}, -L^{JA}-OR^{JA1}, —O-L^{JA}-OR^{JA1}, —NH-L^{JA}-OR^{JA1}, —NR^{JA1}-L^{JA}-OR^{JA1},
—SH, —SR^{JA1},
—CN,
—NO₂,
—NH₂, —NHR^{JA1}, —NR^{JA1}₂, —NR^{JA2}R^{JA3},
-L^{JA}-NH₂, -L^{JA}-NHR^{JA1}, -L^{JA}-NR^{JA1}₂, -L^{JA}-NR^{JA2}R^{JA3},
—O-L^{JA}-NH₂, —O-L^{JA}-NHR^{JA1}, —O-L^{JA}-NR^{JA1}₂, —O-L^{JA}-NR^{JA2}R^{JA3},
—NH-L^{JA}-NH₂, —NR^{JA1}-L^{JA}-NH₂, —NH-L^{JA}-NHR^{JA1}, —NR^{JA1}-L^{JA}-NHR^{JA1},
—NH-L^{JA}-NR^{JA1}₂, —NR^{JA1}-L^{JA}-NR^{JA1}₂,
—NH-L^{JA}-NR^{JA2}R^{JA3}, —NR^{JA1}-L^{JA}-NR^{JA2}R^{JA3},
—OC(═O)R^{JA1},
—C(═O)OH, —C(═O)OR^{JA1},
—C(═O)R^{JA1},
—C(═O)NH₂, —C(═O)NHR^{JA1}, —C(═O)NR^{JA1}₂, —C(═O)NR^{JA2}R^{JA3},
—NHC(═O)R^{JA1}, —NR^{JA1}C(═O)R^{JA1},
—NHC(═O)OR^{JA1}, —NR^{JA1}C(═O)OR^{JA1},
—OC(═O)NH₂, —OC(═O)NHR^{JA1}, —OC(═O)NR^{JA1}₂, —OC(═O)NR^{JA2}R^{JA3},
—NHC(═O)NH₂, —NHC(═O)NHR^{JA1},
—NHC(═O)NR^{JA1}₂, —NHC(═O)NR^{JA2}R^{JA3},
—NR^{JA1}C(═O)NH₂, —NR^{JA1}C(═O)NHR^{JA1},
—NR^{JA1}C(═O)NR^{JA1}₂, —NR^{JA1}C(═O)NR^{JA2}R^{JA3},
—NHS(═O)₂R^{JA1}, —NR^{JA1}S(═O)₂R^{JA1},
—S(═O)₂NH₂, —S(═O)₂NHR^{JA1}, —S(═O)₂NR^{JA1}₂, —S(═O)₂NR^{JA2}R^{JA3},
—S(═O)R^{JA1}, —S(═O)₂R^{JA1}, —OS(═O)₂R^{JA1}, —S(═O)₂OH, —S(═O)₂OR^{JA1},
═O; and
two adjacent groups —R^{S4}, if present, together form —O—CH₂—O— or —O—CH₂CH₂—O—;
   each of —Y^{16A}—, if present, —Y^{16B}—, if present, —Y^{17A}—, if present, —Y^{17B}—, if present, and -L-, if present, is optionally substituted with one or more substituents, —R^{S5}, wherein each —R^{S5} is independently selected from:
—R^{JA1},
—F, —Cl, —Br, —I,
—CF₃, —OCF₃, —SCF₃,
—OH, -L^{JA}-OH, —O-L^{JA}-OH, —NH-L^{JA}-OH, —NR^{JA1}-L^{JA}-OH,
—OR^{JA1}, -L^{JA}-OR^{JA1}, —O-L^{JA}-OR^{JA1}, —NH-L^{JA}-OR^{JA1}, —NR^{JA1}-L^{JA}-OR^{JA1},
—SH, —SR^{JA1},
—CN,
—NO₂,
—NH₂, —NHR^{JA1}, —NR^{JA1}₂, —NR^{JA2}R^{JA3},
-L^{JA}-NH₂, -L^{JA}-NHR^{JA1}, -L^{JA}-NR^{JA1}₂, -L^{JA}-NR^{JA2}R^{JA3},
—O-L^{JA}-NH₂, —O-L^{JA}-NHR^{JA1}, —O-L-NR^{JA1}₂, —O-L^{JA}-NR^{JA2}R^{JA3},
—NH-L^{JA}-NH₂, —NR^{JA1}-L^{JA}-NH₂, —NH-L^{JA}-NHR^{JA1}, —NR^{JA1}-L^{JA}-NHR^{JA1},
—NH-L^{JA}-NR^{JA1}₂, —NR^{JA1}-L^{JA}-NR^{JA1}₂,
—NH-L^{JA}-NR^{JA2}R^{JA3}, —NR^{JA1}-L^{JA}-NR^{JA2}R^{JA3},
—OC(═O)R^{JA1},
—C(═O)OH, —C(═O)OR^{JA1},
—C(═O)R^{JA1},
—C(═O)NH₂, —C(═O)NHR^{JA1}, —C(═O)NR^{JA1}₂, —C(═O)NR^{JA2}R^{JA3},
—NHC(═O)R^{JA1}, —NR^{JA1}C(═O)R^{JA1},
—NHC(═O)OR^{JA1}, —NR^{JA1}C(═O)OR^{JA1},
—OC(═O)NH₂, —OC(═O)NHR^{JA1}, —OC(═O)NR^{JA1}₂, —OC(═O)NR^{JA2}R^{JA3},
—NHC(═O)NH₂, —NHC(═O)NHR^{JA1},
—NHC(═O)NR^{JA1}₂, —NHC(═O)NR^{JA2}R^{JA3},
—NR^{JA1}C(═O)NH₂, —NR^{JA1}C(═O)NHR^{JA1},
—NR^{JA1}C(═O)NR^{JA1}₂, —NR^{JA1}C(═O)NR^{JA2}R^{JA3},
—NHS(═O)₂R^{JA1}, —NR^{JA1}S(═O)₂R^{JA1},
—S(═O)₂NH₂, —S(═O)₂NHR^{JA1}, —S(═O)₂NR^{JA1}₂, —S(═O)₂NR^{JA2}R^{JA3},
—S(═O)R^{JA1}, —S(═O)₂R^{JA1}, —OS(═O)₂R^{JA1}, —S(═O)₂OH, and —S(═O)₂OR^{JA1}, and
═O;
   wherein:
   each -L^{JA}-, if present, is independently saturated aliphatic C₁₋₅alkylene;
   each n is independently 1 to 4;
   each —NR^{JA2}R^{JA3}, if present, is independently C₃₋₁₀heterocyclyl, for example independently piperidino, piperazino, morpholino, oxazepino (e.g. homomorpholino) or diazepino (e.g. homopiperazino), and is optionally substituted, for example, with one or more groups selected from —R^{JJ}, —CF₃, —F, —OH, —OR^{JJ}, —NH₂, —NHR^{JJ}, —NR^{JJ}₂, and ═O; wherein each —R^{JJ} is independently saturated aliphatic C₁₋₄ alkyl;
   each —R^{JA1} is independently:
—R^{JB1}, —R^{JB2}, —R^{JB3}, —R^{JB4}, —R^{JB5}, —R^{JB6}, —R^{JB7}, —R^{JB8}, -L^{JB}-R^{JB4}, -L^{JB}-R^{JB5}, -L^{JB}-R^{JB6},
-L^{JB}-R^{JB7}, or -L^{JB}-R^{JB8};
   each —R^{JB1} is independently saturated aliphatic C₁₋₆alkyl;
   each —R^{JB2} is independently aliphatic C₂₋₆alkenyl;
   each —R^{JB3} is independently aliphatic C₂₋₆alkynyl;
   each —R^{JB4} is independently saturated C₃₋₆cycloalkyl;
   each —R^{JB5} is independently C₃₋₆cycloalkenyl;
   each —R^{JB6} is independently alicyclic C₄₋₇heterocyclyl;
   each —R^{JB7} is independently C₆₋₁₀carboaryl;
   each —R^{JB8} is independently C₅₋₁₀heteroaryl;
   each -L^{JB}- is independently saturated aliphatic C₁₋₃alkylene;
   wherein:
   each —R^{JB4}, —R^{JB5}, —R^{JB6}, —R^{JB7}, and —R^{JB8} is optionally substituted, for example, with one or more substituents —R^{JC1} and/or one or more substituents —R^{JC2},
   each —R^{JB1}, —R^{JB2}R^{JB3}, and -L^{JB}- is optionally substituted, for example, with one or more substituents —R^{JC2}, and
   wherein:
   each —R^{JC1} is independently saturated aliphatic C₁₋₄alkyl, phenyl, or benzyl;
   each —R^{JC2} is independently:
—F, —Cl, —Br, —I,
—CF₃, —OCF₃, —SCF₃,
—OH, -L^{JD}-OH, —O-L^{JD}-OH,
—OR^{JD1}, -L^{JD}-OR^{JD1}, —O-L^{JD}-OR^{JD1},
—SH, —SR^{JD1},
—CN,
—NO₂,
—NH₂, —NHR^{JD1}, —NR^{JD1}₂,
-L^{JD}-NH₂, -L^{JD}-NHR^{JD1}, -L^{JD}-NR^{JD1}₂,
—C(═O)OH, —C(═O)OR^{JD1},
—C(═O)NH₂, —C(═O)NHR^{JD1}, or —C(═O)NR^{JD1}₂;
   wherein:
   each —R^{JA1} is independently saturated aliphatic C₁₋₄ alkyl, phenyl, or benzyl; and
   each -L^{JD}- is independently saturated aliphatic C₁₋₅alkylene.
205. A compound according to paragraph 204, wherein each —R^{S1}, if present, is independently selected from:
   —R^{JA1},
   —F, —Cl, —Br, —I
   —CF₃, —OCF₃,
   —OH, -L^{JA}-OH, —O-L^{JA}-OH,
   —OR^{JA1}, -L^{JA}-OR^{JA1}, —O-L^{JA}-OR^{JA1},
   —CN,
   —NH₂, —NHR^{JA1}, —NR^{JA1}₂,
   -L^{JA}-NH₂, -L^{JA}-NHR^{JA1}, -L^{JA}-NR^{JA1}₂,
   —C(═O)OH, —C(═O)OR^{JA1},
   —C(═O)R^{JA1},
   —C(═O)NH₂, —C(═O)NHR^{JA1}, —C(═O)NR^{JA1}₂, —C(═O)NR^{JA2}R^{JA3},
   —NHC(═O)R^{JA1}, —NR^{JA1}C(═O)R^{JA1},
   —NHC(═O)OR^{JA1}, —NR^{JA1}C(═O)OR^{JA1},
   —S(═O)R^{JA1}, —S(═O)₂R^{JA1}, —OS(═O)₂R^{JA1}, —S(═O)₂OH, —S(═O)₂OR^{JA1}; and
   ═O.
206. A compound according to paragraph 204 or paragraph 205, wherein each —R^{S1}, if present, is independently selected from:
   —R^{JA1},
   —F, —Cl, —Br, —I
   —CF₃,
   —OH, -L^{JA}-OH, —O-L^{JA}-OH,
   —OR^{JA1}, -L^{JA}-OR^{JA1}, —O-L^{JA}-OR^{JA1},
   —CN,
   —NH₂, —NHR^{JA1}, —NR^{JA1}₂, and
   —NHC(═O)OR^{JA1}, —NR^{JA1}C(═O)OR^{JA1}.
207. A compound according to any one of paragraphs 204 to 206, wherein each —R^{S2}, if present, is independently selected from:
   —R^{JA1},
   —F, —Cl, —Br, —I
   —CF₃, —OCF₃,
   —OH, -L^{JA}-OH, —O-L^{JA}-OH,
   —OR^{JA1}, -L^{JA}-OR^{JA1}, —O-L^{JA}-OR^{JA1},
   —CN,
   —NO₂,
   —NH₂, —NHR^{JA1}, —NR^{JA1}₂, —NR^{JA2}R^{JA3},
   -L^{JA}-NH₂, -L^{JA}-NHR^{JA1}, -L^{JA}-NR^{JA1}₂, -L^{JA}-NR^{JA2}R^{JA3},
   —C(═O)OH, —C(═O)OR^{JA1},
   —C(═O)R^{JA1},
   —C(═O)NH₂, —C(═O)NHR^{JA1}, —C(═O)NR^{JA1}₂, —C(═O)NR^{JA2}R^{JA3},
   —NHC(═O)R^{JA1}, —NR^{JA1}C(═O)R^{JA1},
   —S(═O)R^{JA1}, —S(═O)₂R^{JA1}, —OS(═O)₂R^{JA1}, —S(═O)₂OH, —S(═O)₂OR^{JA1};
   ═O; and two adjacent groups —R^{S2}, if present, together form —O—CH₂—O— or —O—CH₂CH₂—O—.
208. A compound according to any one of paragraphs 204 to 207, wherein each —R^{S2}, if present, is independently selected from:
   —R^{JA1},
   —F, —Cl, —Br, —I
   —CF₃,
   —OH, -L^{JA}-OH,
   —OR^{JA1}, -L^{JA}-OR^{JA1},
   —CN,
   —NH₂, —NHR^{JA1}, —NR^{JA1}₂, —NR^{JA2}R^{JA3},
   -L^{JA}-NH₂, -L^{JA}-NHR^{JA1}, -L^{JA}-NR^{JA1}₂,
   —C(═O)OH,
   —C(═O)R^{JA1},
   —C(═O)NH₂, —C(═O)NHR^{JA1}, and —C(═O)NR^{JA1}₂; and
   two adjacent groups —R^{S2}, if present, together form —O—CH₂—O— or —O—CH₂CH₂—O—.
209. A compound according to any one of paragraphs 204 to 208, wherein each —R^{S3}, if present, is independently selected from:
   —R^{JA1},
   —F, —Cl, —Br, —I
   —CF₃, —OCF₃,
   —OH, -L^{JA}-OH, —O-L^{JA}-OH,
   —OR^{JA1}, -L^{JA}-OR^{JA1}, —O-L^{JA}-OR^{JA1},
   —CN,
   —NO₂,
   —NH₂, —NHR^{JA1}, —NR^{JA1}₂, —NR^{JA2}R^{JA3},
   -L^{JA}-NH₂, -L^{JA}-NHR^{JA1}, -L^{JA}-NR^{JA1}₂, -L^{JA}-NR^{JA2}R^{JA3},
   —C(═O)OH, —C(═O)OR^{JA1},
   —C(═O)R^{JA1},
   —C(═O)NH₂, —C(═O)NHR^{JA1}, —C(═O)NR^{JA1}₂, —C(═O)NR^{JA2}R^{JA3},
   —NHC(═O)R^{JA1}, —NR^{JA1}C(═O)R^{JA1},
   —S(═O)R^{JA1}, —S(═O)₂R^{JA1}, —OS(═O)₂R^{JA1}, —S(═O)₂OH, —S(═O)₂OR^{JA1};
   ═O; and
   two adjacent groups —R^{S3}, if present, together form —O—CH₂—O— or —O—CH₂CH₂—O—.
210. A compound according to any one of paragraphs 204 to 209, wherein each —R^{S3}, if present, is independently selected from:
   —R^{JA1},
   —F, —Cl, —Br, —I
   —CF₃,
   —OH, -L^{JA}-OH,
   —OR^{JA1}, -L^{JA}-OR^{JA1},
   —CN,
   —NH₂, —NHR^{JA1}, —NR^{JA1}₂, —NR^{JA2}R^{JA3},
   -L^{JA}-NH₂, -L^{JA}-NHR^{JA1}, -L^{JA}-NR^{JA1}₂,
   —C(═O)OH,
   —C(═O)R^{JA1},
   —C(═O)NH₂, —C(═O)NHR^{JA1}, and —C(═O)NR^{JA1}₂; and
   two adjacent groups —R^{S3}, if present, together form —O—CH₂—O— or —O—CH₂CH₂—O—.
211. A compound according to any one of paragraphs 204 to 210, wherein each —R^{S4}, if present, is independently selected from:
   —R^{JA1},
   —F, —Cl, —Br, —I
   —CF₃, —OCF₃,
   —OH, -L^{JA}-OH, —O-L^{JA}-OH,
   —OR^{JA1}, -L^{JA}-OR^{JA1}, —O-L^{JA}-OR^{JA1},
   —CN,
   —NO₂,
   —NH₂, —NHR^{JA1}, —NR^{JA1}₂, —NR^{JA2}R^{JA3},
   -L^{JA}-NH₂, -L^{JA}-NHR^{JA1}, -L^{JA}-NR^{JA1}₂, -L^{JA}-NR^{JA2}R^{JA3},
   —C(═O)OH, —C(═O)OR^{JA1},
   —C(═O)R^{JA1},
   —C(═O)NH₂, —C(═O)NHR^{JA1}, —C(═O)NR^{JA1}₂, —C(═O)NR^{JA2}R^{JA3},
   —NHC(═O)R^{JA1}, —NR^{JA1}C(═O)R^{JA1},
   —S(═O)R^{JA1}, —S(═O)₂R^{JA1}, —OS(═O)₂R^{JA1}, —S(═O)₂OH, —S(═O)₂OR^{JA1};
   ═O; and
   two adjacent groups —R^{S4}, if present, together form —O—CH₂—O— or —O—CH₂CH₂—O—.
212. A compound according to any one of paragraphs 204 to 211, wherein each —R^{S4}, if present, is independently selected from:
   —R^{JA1},
   —F, —Cl, —Br, —I
   —CF₃,
   —OH, -L^{JA}-OH,
   —OR^{JA1}, -L^{JA}-OR^{JA1},
   —CN,
   —NH₂, —NHR^{JA1}, —NR^{JA1}₂, —NR^{JA2}R^{JA3},
   -L^{JA}-NH₂, -L^{JA}-NHR^{JA1}, -L^{JA}-NR^{JA1}₂,
   —C(═O)OH,
   —C(═O)R^{JA1},
   —C(═O)NH₂, —C(═O)NHR^{JA1}, and —C(═O)NR^{JA1}₂; and
   two adjacent groups —R^{S4}, if present, together form —O—CH₂—O— or —O—CH₂CH₂—O—.
213. A compound according to any one of paragraphs 204 to 212, wherein each —R⁵⁵, if present, is independently selected from:
   —R^{JA1},
   —F, —Cl, —Br, —I
   —CF₃, —OCF₃,
   —OH, -L^{JA}-OH,
   —OR^{JA1}, -L^{JA}-OR^{JA1},
   —CN,
   —NO₂,
   —NH₂, —NHR^{JA1}, —NR^{JA1}₂, —NR^{JA2}R^{JA3},
   —O-L^{JA}-NH₂, —O-L^{JA}-NHR^{JA1}, —O-L^{JA}-NR^{JA1}₂, —O-L^{JA}-NR^{JA2}R^{JA3},
   —OC(═O)R^{JA1},
   —C(═O)OH, —C(═O)OR^{JA1},
   —C(═O)R^{JA1}, and
   —S(═O)₂R^{JA1}.
214. A compound according to any one of paragraphs 204 to 213, wherein each —R^{S5}, if present, is independently selected from:
   —R^{JA1},
   —F, —Cl, —Br, —I
   —CF₃, —OCF₃,
   —OH, -L^{JA}-OH,
   —OR^{JA1},
   —CN, and
   —NH₂, —NHR^{JA1}, —NR^{JA1}₂.
215. A compound according to any one of paragraphs 204 to 214, wherein each -L^{JA}-, if present, is independently —(CH₂)ₙ₂—, wherein n2 is independently 1 to 4.
216. A compound according to any one of paragraphs 204 to 215, wherein each -L^{JA}-, if present, is independently —CH₂— or —CH₂CH₂—.
217. A compound according to any one of paragraphs 204 to 216, wherein each —NR^{JA2}R^{JA3}, if present, is independently piperidino, piperazino, morpholino, oxazepino (e.g. homomorpholino) or diazepino (e.g. homopiperazino), and is optionally substituted, for example, with one or more groups selected from —R^{JJ}, —CF₃, —F, —OH, —OR^{JJ}, —NH₂, —NHR^{JJ}, —NR^{JJ}₂, and ═O; wherein each —R^{JJ} is independently saturated aliphatic C₁₋₄ alkyl.
218. A compound according to any one of paragraphs 204 to 217, wherein each —NR^{JA2}R^{JA3}, if present, is independently piperidino, piperazino, morpholino, oxazepino (e.g. homomorpholino) or diazepino (e.g. homopiperazino), and is optionally substituted, for example, with one or more groups selected from —R^{JJ}; wherein each —R^{JJ} is independently saturated aliphatic C₁₋₄ alkyl.
219. A compound according to any one of paragraphs 204 to 218, wherein each —R^{JA1}, if present, is independently:
   —R^{JB1}, —R^{JB4}, —R^{JB6}, —R^{JB7}, —R^{JB8}, -L^{JB}-R^{JB4}, -L^{JB}-R^{JB6}, -L^{JB}-R^{JB7}, or -L^{JB}- R^{JB8}.
220. A compound according to any one of paragraphs 204 to 219, wherein each —R^{JA1}, if present, is independently:
   —R^{JB1}, —R^{JB7}, —R^{JB8}, -L^{JB}-R^{JB7}, or -L^{JB}-R^{JB8}.
221. A compound according to any one of paragraphs 204 to 220, wherein each —R^{JA1}, if present, is independently:
   —R^{JB1}, —R^{JB7}, or -L^{JB}-R^{JB7}.
222. A compound according to any one of paragraphs 204 to 221, wherein each —R^{JB7} if present, is independently phenyl, and is optionally substituted.
223. A compound according to any one of paragraphs 204 to 222, wherein each —R^{JB8}, if present, is independently C₅₋₆heteroaryl, and is optionally substituted.
224. A compound according to any one of paragraphs 204 to 223, wherein each —R^{JB8}, if present, is independently C₉₋₁₀heteroaryl, and is optionally substituted.
225. A compound according to any one of paragraphs 204 to 224, wherein each -L^{JB}-, if present, is independently —CH₂— or —CH₂CH₂—.
226. A compound according to any one of paragraphs 204 to 225, wherein each -L^{JB}-, if present, is independently —CH₂—.
227. A compound according to any one of paragraphs 204 to 226, wherein each —R^{JC1}, if present, is independently saturated aliphatic C₁₋₄ alkyl.
228. A compound according to any one of paragraphs 204 to 227, wherein each —R^{JC2} is independently:
   —F, —Cl, —Br, —I,
   —OH,
   —OR^{JD1},
   —CN,
   —NO₂,
   —NH₂, —NHR^{JD1}, or —NR^{JD1}₂.
229. A compound according to any one of paragraphs 204 to 228, wherein each —R^{JD1}, if present, is independently saturated aliphatic C₁₋₄ alkyl.
230. A compound according to any one of paragraphs 204 to 229, wherein each -L^{JD}-, if present, is independently —(CH₂)ₘ₂—, wherein m2 is independently 1 to 4.
231. A compound according to any one of paragraphs 204 to 230, wherein each -L^{JD}-, if present, is independently —CH₂— or —CH₂CH₂—.
232. A compound according to any one of paragraphs 1 to 231, wherein —R^{17A} is —Y^{17A}Z^{17A}, —Y^{17A} is —CH₂—, —CH(CH₃)— or —C(O)— and Z^{17A} is independently phenyl, and is optionally substituted.
233. A compound according to any one of paragraphs 1 to 232, wherein —R^{17A} is —Y^{17A}Z^{17A}, —Y^{17A} is —CH₂— and Z^{17A} is independently phenyl, and is optionally substituted.
234. A compound according to any one of paragraphs 1 to 233, wherein —Z^{17A} is independently phenyl, and is optionally substituted.
235. A compound according to any one of paragraphs 1 to 234, wherein —Z^{17A} is independently 4-substituted phenyl, and is optionally further substituted.
236. A compound according to any one of paragraphs 1 to 235, wherein —Z^{17A} is independently 4-substituted phenyl wherein the 4-substituent is chloro, fluoro, bromo, methyl, ^{t}butyl, methoxy, CF₃, amino, piperzino, morpholino, piperidino, diazepino (preferably homopiperazino) or oxazepino (preferably homomorpholino).
237. A compound according to any one of paragraphs 1 to 236, wherein —Z^{17A} is independently 4-substituted phenyl wherein the 4-substituent is chloro, fluoro, bromo, methoxy, or amino.
238. A compound according to any one of paragraphs 1 to 237, wherein —Z^{17A} is independently 4-substituted phenyl wherein the 4-substituent is chloro, fluoro, bromo or methoxy.
239. A compound according to any one of paragraphs 1 to 238, wherein —Z^{17A} is independently 4-substituted phenyl wherein the 4-substituent is chloro, fluoro or bromo.
240. A compound according to any one of paragraphs 1 to 239, wherein —Z^{17A} is independently 4-substituted phenyl wherein the 4-substituent is chloro or fluoro.
241. A compound according to any one of paragraphs 1 to 240, wherein —Z^{17A} is independently 4-chloro substituted phenyl.
242. A compound according to any one of paragraphs 1 to 241, wherein —Z^{17A} is independently 2,4-substituted or 3,4-substituted phenyl.
243. A compound according to any one of paragraphs 1 to 242, wherein —Z^{17A} is independently 2,4-substituted phenyl.
244. A compound according to any one of paragraphs 1 to 243, wherein —R^{17A} is independently selected from the group consisting of:

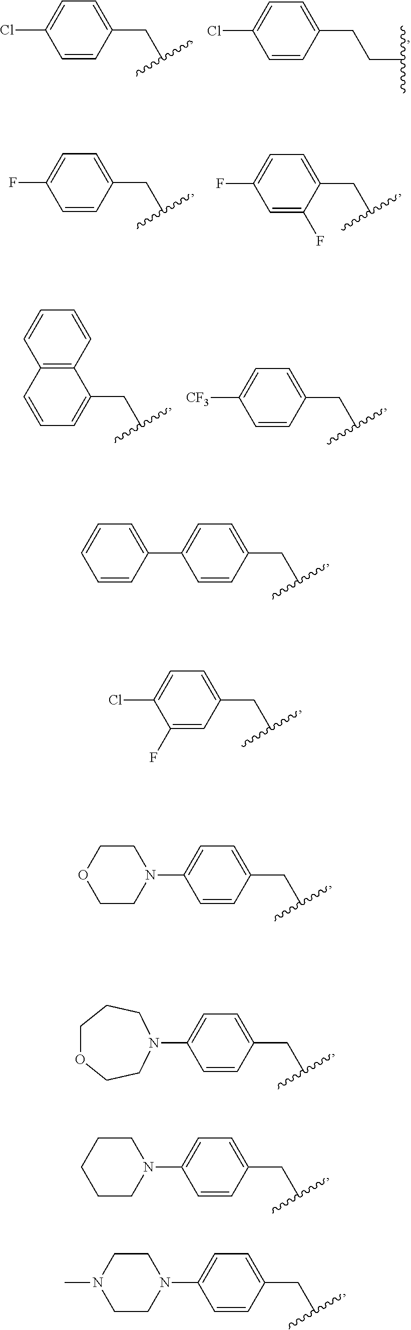

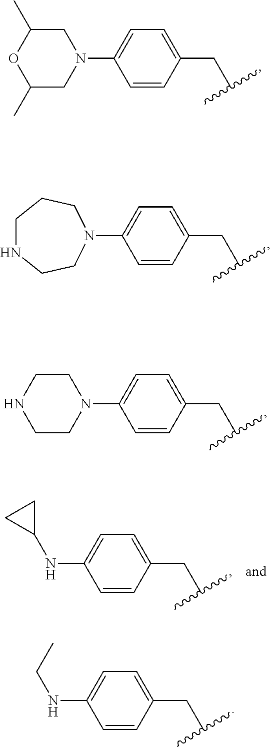

245. A compound according to any one of paragraphs 1 to 244, wherein —X¹⁶ is independently —OH.
246. A compound according to paragraph 1, selected from the following compounds, and pharmaceutically acceptable salts, hydrates, and solvates thereof: Compound Nos. MC-001 through MC-059.
247. A compound according to paragraph 1, selected from the following compounds, and pharmaceutically acceptable salts, hydrates, and solvates thereof: MC-016, MC-016a, MC-016b, MC-004, MC-004a, MC-004b, MC-005, MC-006, MC-006c, MC-006a, MC-059, MC-007, MC-007b, MC-007c, MC-046, MC-046a, MC-046b, MC-014, MC-014a, MC-014b, MC-018, MC-018a, MC-027, MC-027a, MC-027b, MC-028, MC-028a, MC-28b, MC-026, MC-026a, MC-026b, MC-026c, MC-026d, MC-030, MC-030a, MC-030b, MC-029, MC-031, MC-032, MC-056, MC-056a, MC-056b, MC-056c, MC-033, MC-037, MC-037d, MC-055, MC-055a, MC-055b, MC-039, MC-041, MC-041a, MC-042, MC-042a, MC-042b, MC-042c, MC-043, MC-043a, MC-043b, MC-043c, MC-044, MC-044a, MC-044b, MC-012, MC-012a, MC-001, MC-047, MC-047a, MC-047b, MC-052, MC-045, MC-053, MC-051, MC-050, MC-050a, MC-050b, MC-017, MC-017b, MC-017a, MC-034, MC-058 and MC-58b.
248. A pharmaceutical composition comprising a compound according to any one of paragraphs 1 to 247, and a pharmaceutically acceptable carrier, diluent, or excipient.
249. A method of preparing a pharmaceutical composition comprising the step of admixing a compound according to any one of paragraphs 1 to 247, and a pharmaceutically acceptable carrier, diluent, or excipient.
250. A compound according to any one of paragraphs 1 to 247, for use in a method of treatment of the human or animal body by therapy.
251. A compound according to any one of paragraphs 1 to 247, for use in a method of treatment of a disease or condition that is ameliorated by regulating cell proliferation and/or promoting apoptosis.
252. A compound according to any one of paragraphs 1 to 247, for use in a method of treatment of a proliferative condition.
253. A compound according to any one of paragraphs 1 to 247, for use in a method of treatment of cancer.
254. A compound according to any one of paragraphs 1 to 247, for use in a method of treatment of a solid tumour cancer.
255. A compound according to any one of paragraphs 1 to 247, for use in a method of treatment of: lung cancer, breast cancer, ovarian cancer, colorectal cancer, melanoma, or glioma.
256. A compound according to any one of paragraphs 250 to 255, wherein the treatment further comprises treatment with one or more other agents selected from: (a) a DNA topoisomerase I or II inhibitor; (b) a DNA damaging agent; (c) an antimetabolite or TS inhibitor; (d) a microtubule targeted agent; and (e) ionising radiation.
257. A compound according to any one of paragraphs 1 to 247, for use in the manufacture of a medicament for the treatment of a disease or condition that is ameliorated by regulating cell proliferation and/or promoting apoptosis.
258. A compound according to any one of paragraphs 1 to 247, for use in the manufacture of a medicament for the treatment of a proliferative condition.
259. A compound according to any one of paragraphs 1 to 247, for use in the manufacture of a medicament for the treatment of cancer.
260. A compound according to any one of paragraphs 1 to 247, for use in the manufacture of a medicament for the treatment of a solid tumour cancer.
261. A compound according to any one of paragraphs 1 to 247, for use in the manufacture of a medicament for the treatment of: lung cancer, breast cancer, ovarian cancer, colorectal cancer, melanoma, or glioma.
262. A compound according to any one of paragraphs 257 to 261, wherein the treatment further comprises treatment with one or more other agents selected from: (a) a DNA topoisomerase I or II inhibitor; (b) a DNA damaging agent; (c) an antimetabolite or TS inhibitor; (d) a microtubule targeted agent; and (e) ionising radiation.
263. A method of treatment of a disease or condition that is ameliorated by regulating cell proliferation and/or promoting apoptosis comprising administering to a subject in need of treatment a therapeutically effective amount of a compound according to any one of paragraphs 1 to 247.
264. A method of treatment of a proliferative condition comprising administering to a subject in need of treatment a therapeutically effective amount of a compound according to any one of paragraphs 1 to 247.
265. A method of treatment of cancer comprising administering to a subject in need of treatment a therapeutically effective amount of a compound according to any one of paragraphs 1 to 247.
266. A method of treatment of a solid tumour cancer comprising administering to a subject in need of treatment a therapeutically effective amount of a compound according to any one of paragraphs 1 to 247.
267. A method of treatment of lung cancer, breast cancer, ovarian cancer, colorectal cancer, melanoma, or glioma, comprising administering to a subject in need of treatment a therapeutically effective amount of a compound according to any one of paragraphs 1 to 247.
268. A method according to any one of paragraphs 263 to 267, wherein the treatment further comprises administering to the subject one or more other agents selected from: (a) a DNA topoisomerase I or II inhibitor; (b) a DNA damaging agent; (c) an antimetabolite or TS inhibitor; (d) a microtubule targeted agent; and (e) ionising radiation.
269. A method of regulating cell proliferation and/or promoting apoptosis, in vitro or in vivo, comprising contacting a cell with an effective amount of a compound according to any one of paragraphs 1 to 247.

Combinations

Each and every compatible combination of the embodiments described above is explicitly disclosed herein, as if each and every combination was individually and explicitly recited.

### Examples of Specific Embodiments

In one embodiment, the compounds are selected from compounds of the following formulae and pharmaceutically acceptable salts, hydrates, and solvates thereof:

| Compound | Structure |
|---|---|
| MC-001 | 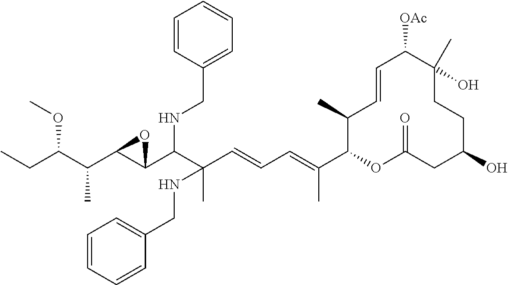 |
| MC-003 | 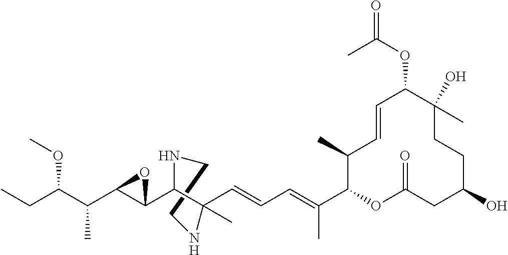 |
| MC-004 | 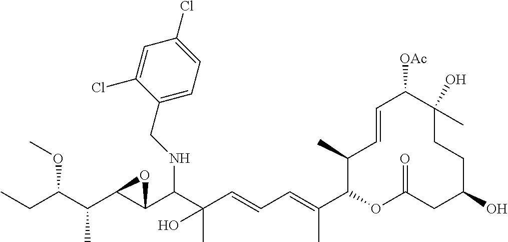 |
| MC-005 | 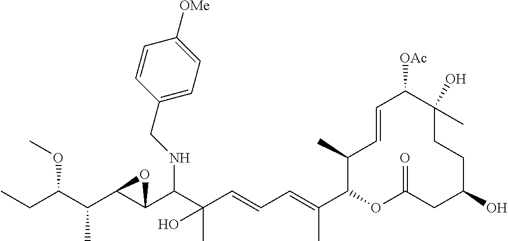 |
| MC-006 | 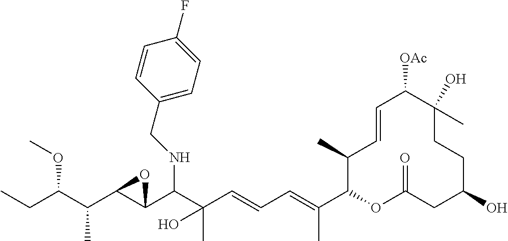 |
| MC-007 | 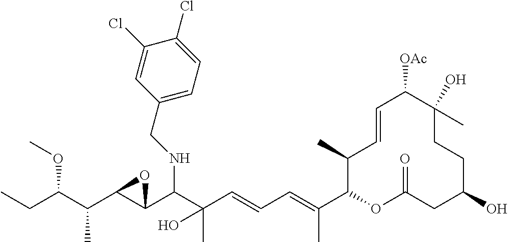 |
| MC-008 | 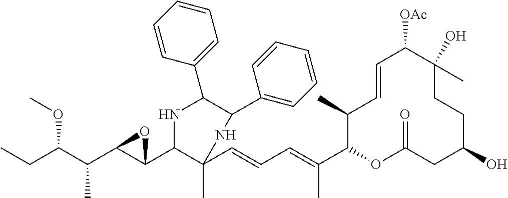 |
| MC-009 | 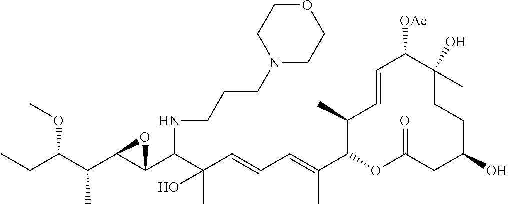 |
| MC-010 | 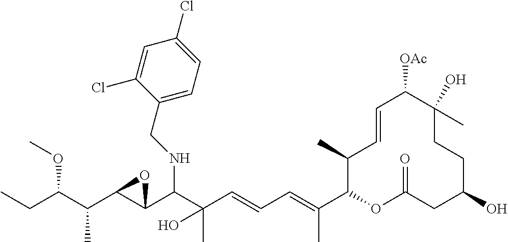 |
| MC-011 | 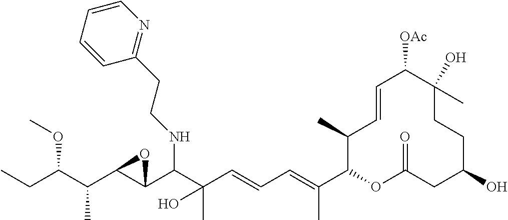 |
| MC-012 | 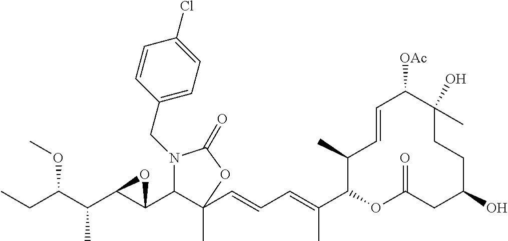 |
| MC-013 | 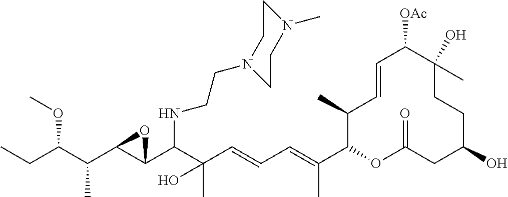 |
| MC-014 | 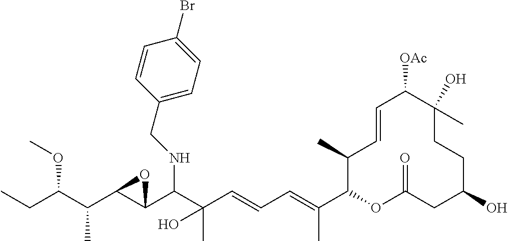 |
| MC-015 | 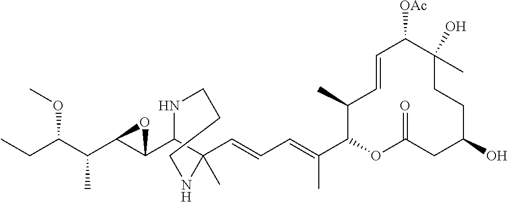 |
| MC-016 | 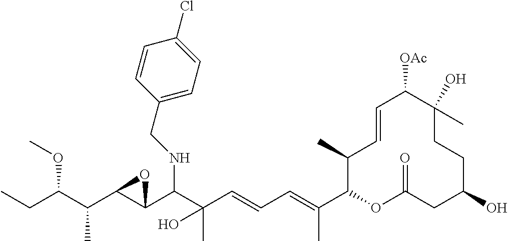 |
| MC-017 | 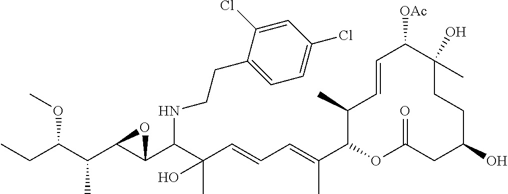 |
| MC-018 | 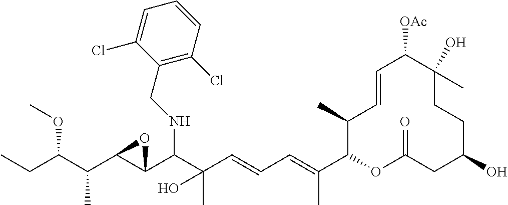 |
| MC-019 | 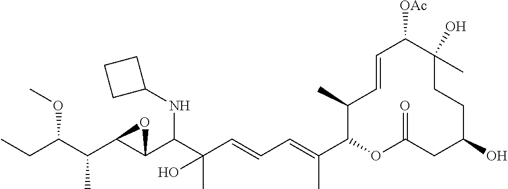 |
| MC-020 | 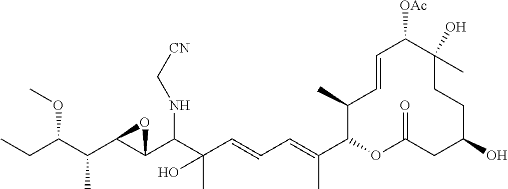 |
| MC-021 | 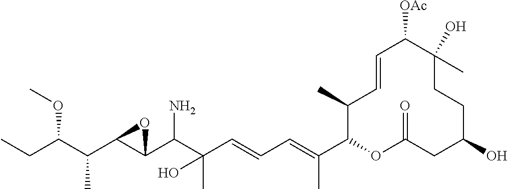 |
| MC-022 | 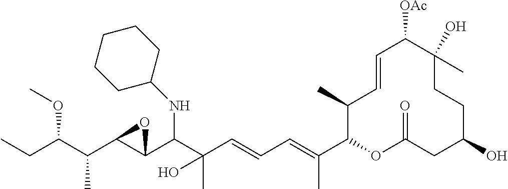 |
| MC-023 | 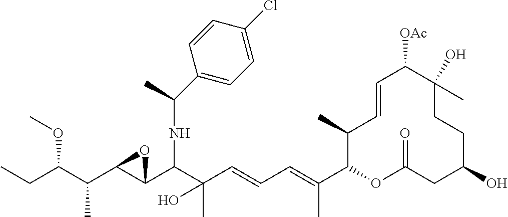 |
| MC-024 | 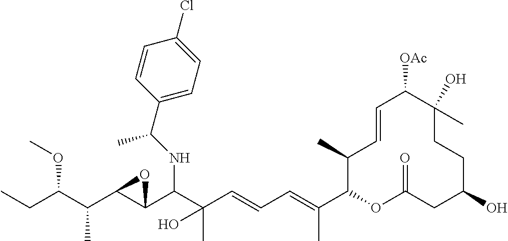 |
| MC-025 | 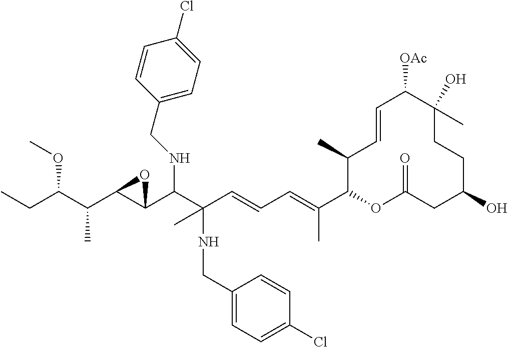 |
| MC-026 | 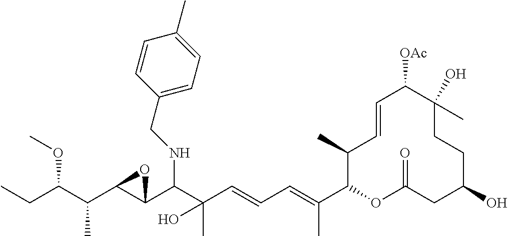 |
| MC-027 | 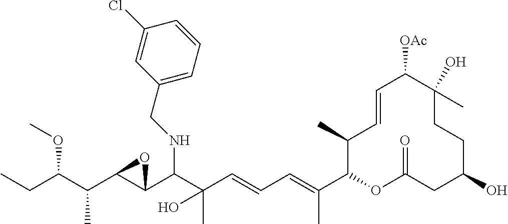 |
| MC-028 | 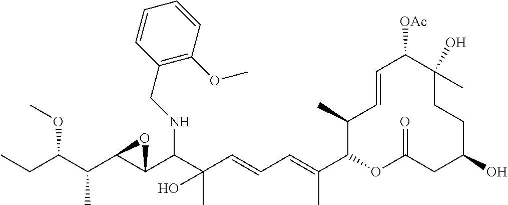 |
| MC-029 | 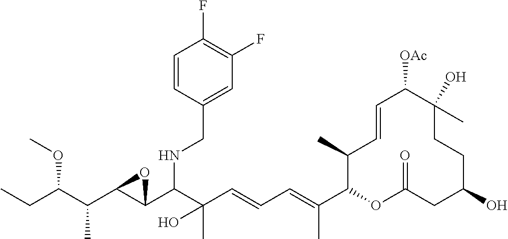 |
| MC-030 | 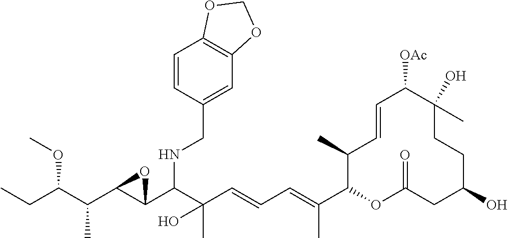 |
| MC-031 | 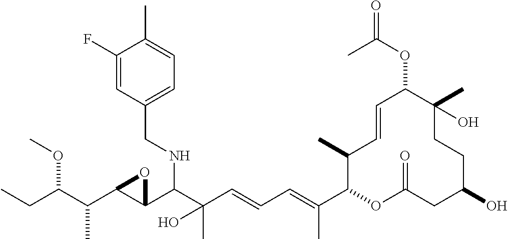 |
| MC-032 | 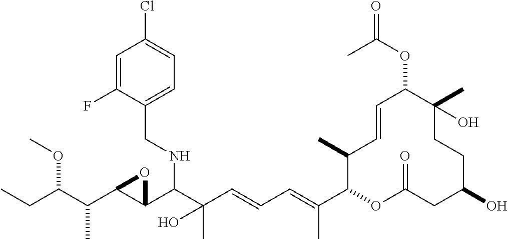 |
| MC-033 | 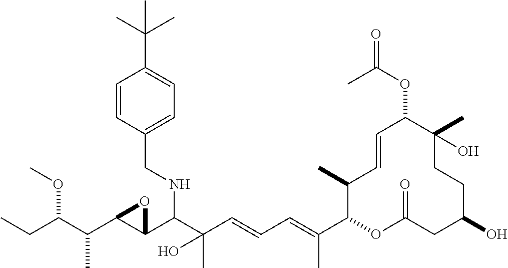 |
| MC-034 | 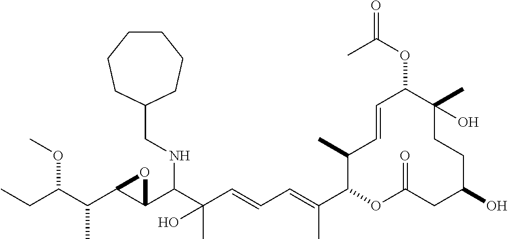 |
| MC-035 | 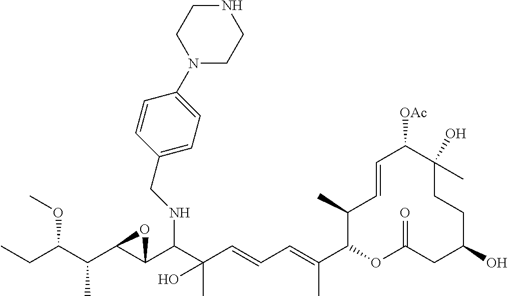 |
| MC-036 | 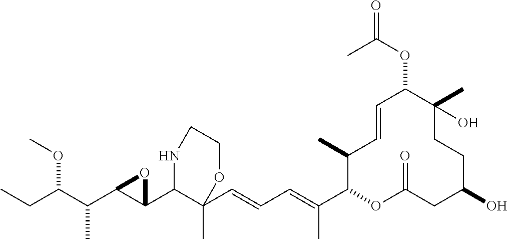 |
| MC-037 | 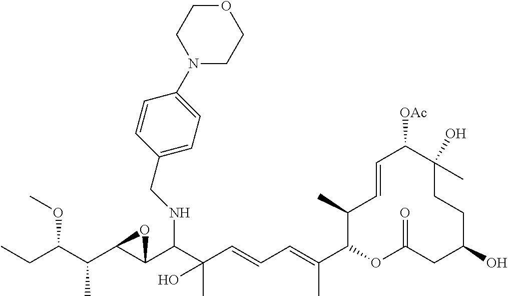 |
| MC-038 | 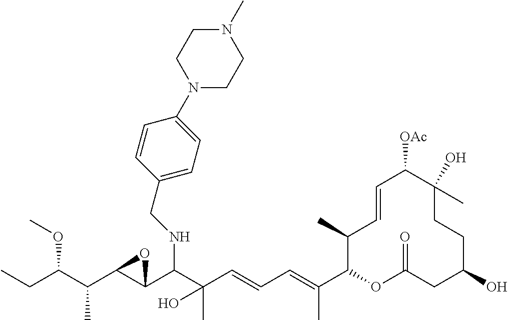 |
| MC-039 | 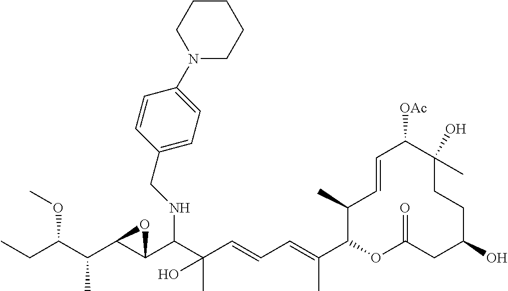 |
| MC-040 | 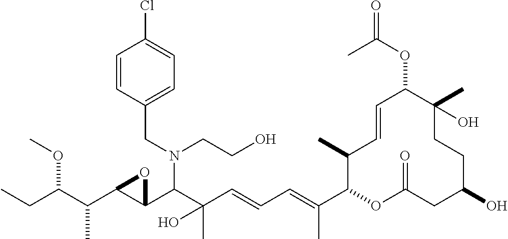 |
| MC-041 | 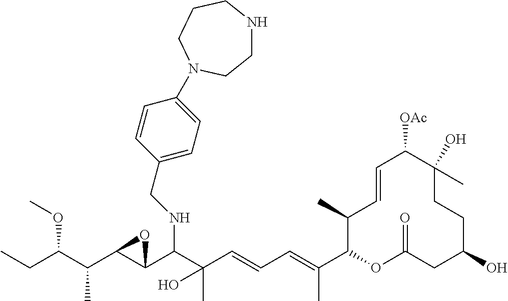 |
| MC-042 | 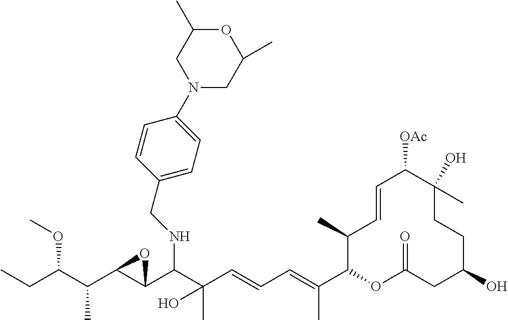 |
| MC-043 | 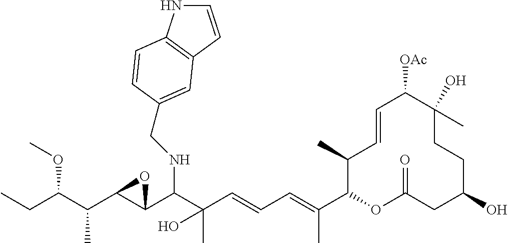 |
| MC-044 | 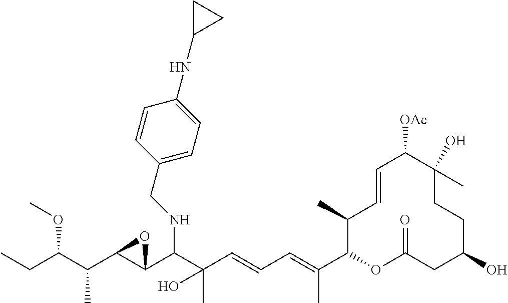 |
| MC-045 | 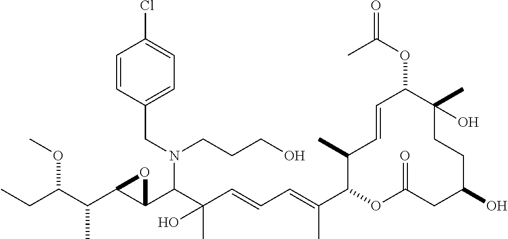 |
| MC-046 | 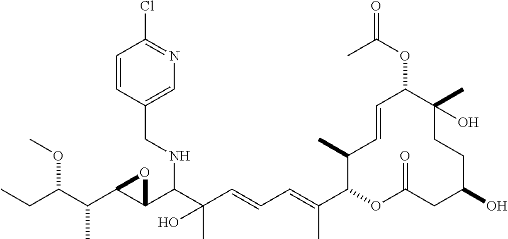 |
| MC-047 | 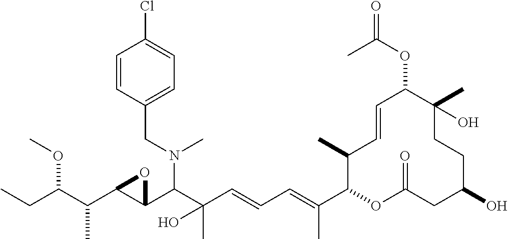 |
| MC-048 | 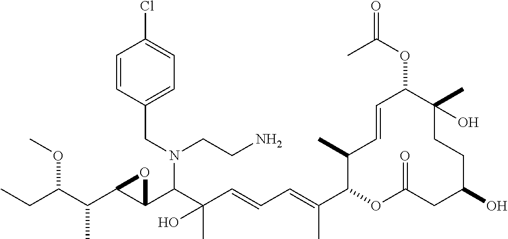 |
| MC-049 | 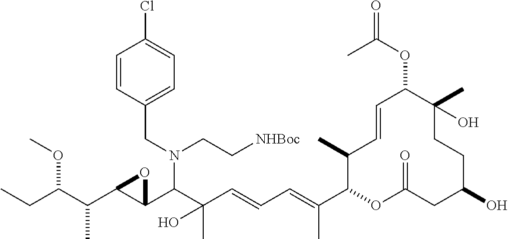 |
| MC-050 | 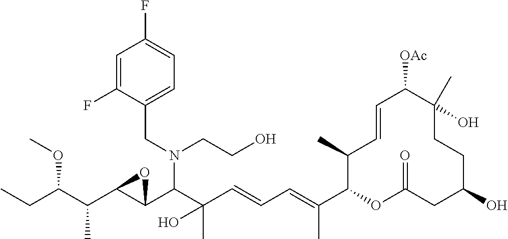 |
| MC-051 | 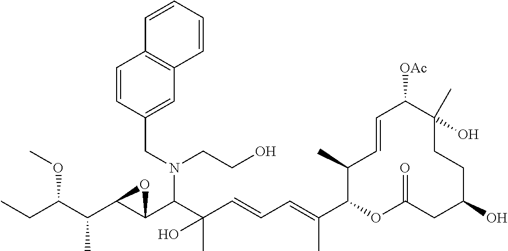 |
| MC-052 | 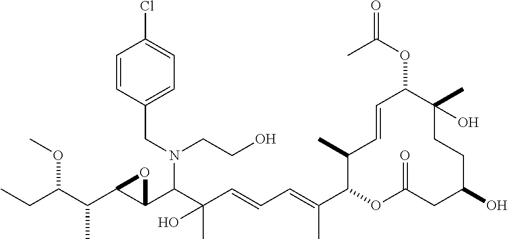 |
| MC-053 | 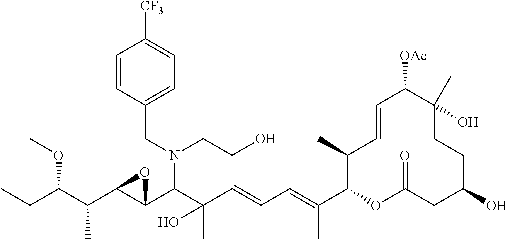 |
| MC-054 | 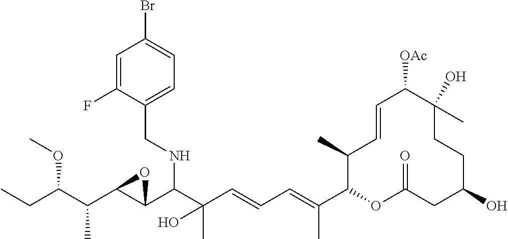 |
| MC-055 | 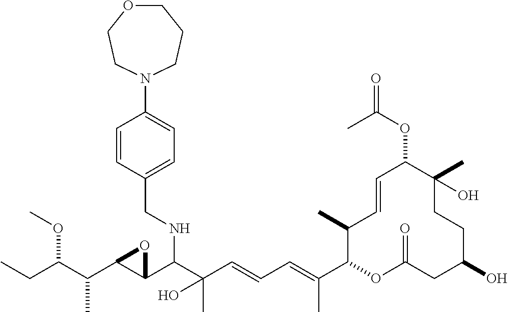 |
| MC-056 | 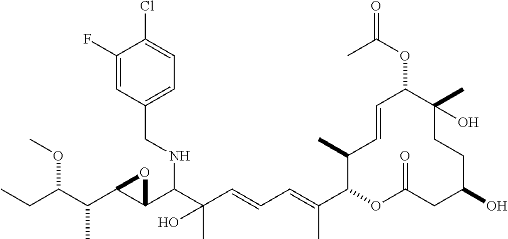 |
| MC-057 | 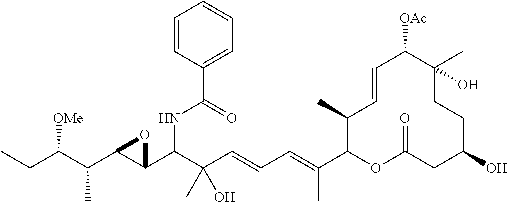 |
| MC-058 | 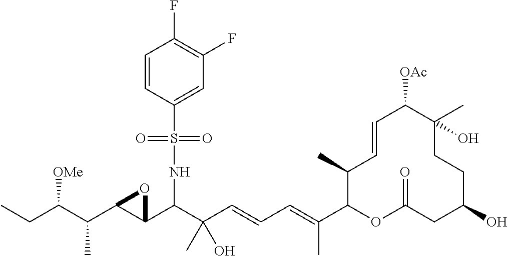 |
| MC-059 | 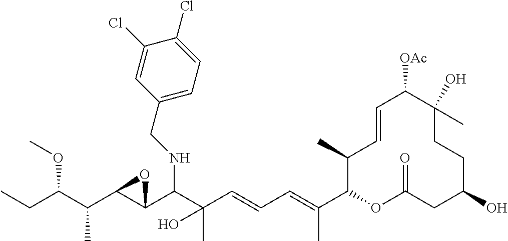 |

In one embodiment, the compounds are selected from the group consisting of: MC-016, MC-004, MC-005, MC-006, MC-059, MC-007, MC-046, MC-014, MC-018, MC-027, MC-028, MC-026, MC-030, MC-029, MC-031, MC-032, MC-056, MC-033, MC-037, MC-055, MC-039, MC-041, MC-042, MC-043, MC-044, MC-012, MC-001, MC-047, MC-052, MC-045, MC-053, MC-051, MC-050, MC-017, MC-034, and MC-058.

In one embodiment, the compounds are selected from the group consisting of: MC-016, MC-004, MC-005, MC-006, MC-059, MC-046, MC-014, MC-028, MC-026, MC-030, MC-029, MC-031, MC-032, MC-056, MC-033, MC-037, MC-055, MC-039, MC-042, MC-043, MC-047, and MC-052.

In embodiments, the MC compounds are diastereomers. As used herein, the suffix “a”, “b”, etc, when applied to the compound name “MC-XXX” indicates a diastereomer. Certain diastereomers are discussed below.

In one embodiment, the compounds are selected from the group consisting of: MC-016, MC-016a, MC-016b, MC-004, MC-004a, MC-004b, MC-005, MC-006, MC-006c, MC-006a, MC-059, MC-007, MC-007b, MC-007c, MC-046, MC-046a, MC-046b, MC-014, MC-014a, MC-014b, MC-018, MC-018a, MC-027, MC-027a, MC-027b, MC-028, MC-028a, MC-28b, MC-026, MC-026a, MC-026b, MC-026c, MC-026d, MC-030, MC-030a, MC-030b, MC-029, MC-031, MC-032, MC-056, MC-056a, MC-056b, MC-056c, MC-033, MC-037, MC-037d, MC-055, MC-055a, MC-055b, MC-039, MC-041, MC-041a, MC-042, MC-042a, MC-042b, MC-042c, MC-043, MC-043a, MC-043b, MC-043c, MC-044, MC-044a, MC-044b, MC-012, MC-012a, MC-001, MC-047, MC-047a, MC-047b, MC-052, MC-045, MC-053, MC-051, MC-050, MC-050a, MC-050b, MC-017, MC-017b, MC-017a, MC-034, MC-058 and MC-58b.

In one embodiment, the compounds are selected from the group consisting of: MC-016, MC-016a, MC-016b, MC-004, MC-004a, MC-004b, MC-005, MC-006, MC-006c, MC-006a, MC-059, MC-046, MC-046a, MC-046b, MC-014, MC-014a, MC-014b, MC-028, MC-28b, MC-026, MC-026a, MC-026b, MC-026c, MC-026d, MC-030, MC-030a, MC-030b, MC-029, MC-031, MC-032, MC-056, MC-056a, MC-056b, MC-056c, MC-033, MC-037, MC-037d, MC-05, MC-055a, MC-039, MC-042, MC-042a, MC-043, MC-043a, MC-047, MC-047a, MC-047b and MC-052.

MC compounds, as described herein, exhibit anticancer activity, for example against the test strains described herein. Suitably the MC compounds, as described herein, exhibit good levels of stability, particularly solution stability, for example as demonstrated in the assays reported herein.

MC compounds, as described herein, preferably exhibit improved performance as compared to known macrolide compounds in terms of one or more of pharmacokinetics (including one or more of adsorption, distribution, metabolism and excretion), pharmacodynamics, bioavailability, toxicity, solubility and pharmacological activity.

Substantially Purified Forms

One aspect of the present invention pertains to MC compounds, as described herein, in substantially purified form and/or in a form substantially free from contaminants.

In one embodiment, the substantially purified form is at least 50% by weight, e.g., at least 60% by weight, e.g., at least 70% by weight, e.g., at least 80% by weight, e.g., at least 90% by weight, e.g., at least 95% by weight, e.g., at least 97% by weight, e.g., at least 98% by weight, e.g., at least 99% by weight.

Unless specified, the substantially purified form refers to the compound in any stereoisomeric or enantiomeric form. For example, in one embodiment, the substantially purified form refers to a mixture of stereoisomers, i.e., purified with respect to other compounds. In one embodiment, the substantially purified form refers to one stereoisomer, e.g., optically pure stereoisomer. In one embodiment, the substantially purified form refers to a mixture of enantiomers. In one embodiment, the substantially purified form refers to a equimolar mixture of enantiomers (i.e., a racemic mixture, a racemate). In one embodiment, the substantially purified form refers to one enantiomer, e.g., optically pure enantiomer.

In one embodiment, the contaminants represent no more than 50% by weight, e.g., no more than 40% by weight, e.g., no more than 30% by weight, e.g., no more than 20% by weight, e.g., no more than 10% by weight, e.g., no more than 5% by weight, e.g., no more than 3% by weight, e.g., no more than 2% by weight, e.g., no more than 1% by weight.

Unless specified, the contaminants refer to other compounds, that is, other than stereoisomers or enantiomers. In one embodiment, the contaminants refer to other compounds and other stereoisomers. In one embodiment, the contaminants refer to other compounds and the other enantiomer.

In one embodiment, the substantially purified form is at least 60% optically pure (i.e., 60% of the compound, on a molar basis, is the desired stereoisomer or enantiomer, and 40% is the undesired stereoisomer or enantiomer), e.g., at least 70% optically pure, e.g., at least 80% optically pure, e.g., at least 90% optically pure, e.g., at least 95% optically pure, e.g., at least 97% optically pure, e.g., at least 98% optically pure, e.g., at least 99% optically pure.

Chirality

In some embodiments, the compound may have one or more chiral centres.

The chiral centre, or each chiral centre, if more than one is present, is independently in the R-configuration or the S-configuration.

If no configuration is indicated, then both configurations are encompassed.

Isomers

Certain compounds may exist in one or more particular geometric, optical, enantiomeric, diasteriomeric, epimeric, atropic, stereoisomeric, tautomeric, conformational, or anomeric forms, including but not limited to, cis- and trans-forms; E- and Z-forms; c-, t-, and r-forms; endo- and exo-forms; R—, S—, and meso-forms; D- and L-forms; d- and I-forms; (+) and (−) forms; keto-, enol-, and enolate-forms; syn- and anti-forms; synclinal- and anticlinal-forms; α- and β-forms; axial and equatorial forms; boat-, chair-, twist-, envelope-, and halfchair-forms; and combinations thereof, hereinafter collectively referred to as “isomers” (or “isomeric forms”).

Note that, except as discussed below for tautomeric forms, specifically excluded from the term “isomers,” as used herein, are structural (or constitutional) isomers (i.e., isomers which differ in the connections between atoms rather than merely by the position of atoms in space). For example, a reference to a methoxy group, —OCH₃, is not to be construed as a reference to its structural isomer, a hydroxymethyl group, —CH₂OH. Similarly, a reference to ortho-chlorophenyl is not to be construed as a reference to its structural isomer, meta-chlorophenyl. However, a reference to a class of structures may well include structurally isomeric forms falling within that class (e.g., C₁₋₇alkyl includes n-propyl and iso-propyl; butyl includes n-, iso-, sec-, and tert-butyl; methoxyphenyl includes ortho-, meta-, and para-methoxyphenyl).

The above exclusion does not pertain to tautomeric forms, for example, keto-, enol-, and enolate-forms, as in, for example, the following tautomeric pairs: keto/enol (illustrated below), imine/enamine, amide/imino alcohol, amidine/amidine, nitroso/oxime, thioketone/enethiol, N-nitroso/hydroxyazo, and nitro/aci-nitro.

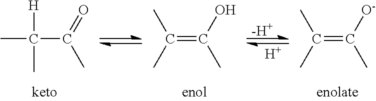

Note that specifically included in the term “isomer” are compounds with one or more isotopic substitutions. For example, H may be in any isotopic form, including ¹H, ²H(D), and ³H(T); C may be in any isotopic form, including ¹²C, ¹³C, and ¹⁴C; O may be in any isotopic form, including ¹⁶O and ¹⁸O; and the like.

Unless otherwise specified, a reference to a particular compound includes all such isomeric forms, including mixtures (e.g., racemic mixtures) thereof. Methods for the preparation (e.g., asymmetric synthesis) and separation (e.g., fractional crystallisation and chromatographic means) of such isomeric forms are either known in the art or are readily obtained by adapting the methods taught herein, or known methods, in a known manner.

Salts

It may be convenient or desirable to prepare, purify, and/or handle a corresponding salt of the compound, for example, a pharmaceutically-acceptable salt. Examples of pharmaceutically acceptable salts are discussed in Berge et al., 1977, “Pharmaceutically Acceptable Salts,” J. Pharm. Sci., Vol. 66, pp. 1-19.

For example, if the compound is anionic, or has a functional group which may be anionic (e.g., —COOH may be —COO⁻), then a salt may be formed with a suitable cation. Examples of suitable inorganic cations include, but are not limited to, alkali metal ions such as Na⁺ and K⁺, alkaline earth cations such as Ca²⁺ and Mg²⁺, and other cations such as Al⁺³. Examples of suitable organic cations include, but are not limited to, ammonium ion (i.e., NH₄⁺) and substituted ammonium ions (e.g., NH₃R⁺, NH₂R₂⁺, NHR₃⁺, NR₄⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

If the compound is cationic, or has a functional group which may be cationic (e.g., —NH₂ may be —NH₃⁺), then a salt may be formed with a suitable anion. Examples of suitable inorganic anions include, but are not limited to, those derived from the following inorganic acids: hydrochloric, hydrobromic, hydroiodic, sulfuric, sulfurous, nitric, nitrous, phosphoric, and phosphorous.

Examples of suitable organic anions include, but are not limited to, those derived from the following organic acids: 2-acetyoxybenzoic, acetic, ascorbic, aspartic, benzoic, camphorsulfonic, cinnamic, citric, edetic, ethanedisulfonic, ethanesulfonic, fumaric, glucheptonic, gluconic, glutamic, glycolic, hydroxymaleic, hydroxynaphthalene carboxylic, isethionic, lactic, lactobionic, lauric, maleic, malic, methanesulfonic, mucic, oleic, oxalic, palmitic, pamoic, pantothenic, phenylacetic, phenylsulfonic, propionic, pyruvic, salicylic, stearic, succinic, sulfanilic, tartaric, toluenesulfonic, and valeric. Examples of suitable polymeric organic anions include, but are not limited to, those derived from the following polymeric acids: tannic acid, carboxymethyl cellulose.

Unless otherwise specified, a reference to a particular compound also includes salt forms thereof.

Solvates and Hydrates

It may be convenient or desirable to prepare, purify, and/or handle a corresponding solvate of the compound. The term “solvate” is used herein in the conventional sense to refer to a complex of solute (e.g., compound, salt of compound) and solvent. If the solvent is water, the solvate may be conveniently referred to as a hydrate, for example, a mono-hydrate, a di-hydrate, a tri-hydrate, etc.

Unless otherwise specified, a reference to a particular compound also includes solvate and hydrate forms thereof.

Chemically Protected Forms

It may be convenient or desirable to prepare, purify, and/or handle the compound in a chemically protected form. The term “chemically protected form” is used herein in the conventional chemical sense and pertains to a compound in which one or more reactive functional groups are protected from undesirable chemical reactions under specified conditions (e.g., pH, temperature, radiation, solvent, and the like). In practice, well known chemical methods are employed to reversibly render unreactive a functional group, which otherwise would be reactive, under specified conditions. In a chemically protected form, one or more reactive functional groups are in the form of a protected or protecting group (also known as a masked or masking group or a blocked or blocking group). By protecting a reactive functional group, reactions involving other unprotected reactive functional groups can be performed, without affecting the protected group; the protecting group may be removed, usually in a subsequent step, without substantially affecting the remainder of the molecule. See, for example, Protective Groups in Organic Synthesis (T. Green and P. Wuts; 4th Edition; John Wiley and Sons, 2006).

A wide variety of such “protecting,” “blocking,” or “masking” methods are widely used and well known in organic synthesis. For example, a compound which has two nonequivalent reactive functional groups, both of which would be reactive under specified conditions, may be derivatized to render one of the functional groups “protected,” and therefore unreactive, under the specified conditions; so protected, the compound may be used as a reactant which has effectively only one reactive functional group. After the desired reaction (involving the other functional group) is complete, the protected group may be “deprotected” to return it to its original functionality.

For example, a hydroxy group may be protected as an ether (—OR) or an ester (—OC(═O)R), for example, as: a t-butyl ether; a benzyl, benzhydryl (diphenylmethyl), or trityl (triphenylmethyl)ether; a trimethylsilyl or t-butyldimethylsilyl ether; or an acetyl ester (—OC(═O)CH₃, —OAc).

For example, an aldehyde or ketone group may be protected as an acetal (R—CH(OR)₂) or ketal (R₂C(OR)₂), respectively, in which the carbonyl group (>C═O) is converted to a diether (>C(OR)₂), by reaction with, for example, a primary alcohol. The aldehyde or ketone group is readily regenerated by hydrolysis using a large excess of water in the presence of acid.

For example, an amine group may be protected, for example, as an amide (—NRCO—R) or a urethane (—NRCO—OR), for example, as: a methyl amide (—NHCO—CH₃); a benzyloxy amide (—NHCO—OCH₂C₆H₅, —NH-Cbz); as a t-butoxy amide (—NHCO—OC(CH₃)₃, —NH-Boc); a 2-biphenyl-2-propoxy amide (—NHCO—OC(CH₃)₂C₆H₄C₆H₅, —NH-Bpoc), as a 9-fluorenylmethoxy amide (—NH-Fmoc), as a 6-nitroveratryloxy amide (—NH-Nvoc), as a 2-trimethylsilylethyloxy amide (—NH-Teoc), as a 2,2,2-trichlororthyloxy amide (—NH-Troc), as an allyloxy amide (—NH-Alloc), as a 2(-phenylsulfonyl)ethyloxy amide (—NH-Psec); or, in suitable cases (e.g., cyclic amines), as a nitroxide radical (>N—O).

For example, a carboxylic acid group may be protected as an ester for example, as: an C₁₋₇alkyl ester (e.g., a methyl ester; a t-butyl ester); a C₁₋₇haloalkyl ester (e.g., a C₁₋₇trihaloalkyl ester); a triC₁₋₇alkylsilyl-C₁₋₇alkyl ester; or a C₅₋₂₀aryl-C₁₋₇alkyl ester (e.g., a benzyl ester; a nitrobenzyl ester); or as an amide, for example, as a methyl amide.

For example, a thiol group may be protected as a thiorther (—SR), for example, as: a benzyl thiorther; an acetamidomethyl ether (—S—CH₂NHC(═O)CH₃).

Prodrugs

It may be convenient or desirable to prepare, purify, and/or handle the compound in the form of a prodrug. The term “prodrug,” as used herein, pertains to a compound which, when metabolised (e.g., in vivo), yields the desired active compound. Typically, the prodrug is inactive, or less active than the desired active compound, but may provide advantageous handling, administration, or metabolic properties.

For example, some prodrugs are esters of the active compound (e.g., a physiologically acceptable metabolically labile ester). During metabolism, the ester group (—C(═O)OR) is cleaved to yield the active drug. Such esters may be formed by esterification, for example, of any of the carboxylic acid groups (—C(═O)OH) in the parent compound, with, where appropriate, prior protection of any other reactive groups present in the parent compound, followed by deprotection if required.

Also, some prodrugs are activated enzymatically to yield the active compound, or a compound which, upon further chemical reaction, yields the active compound (for example, as in ADEPT, GDEPT, LIDEPT, etc.). For example, the prodrug may be a sugar derivative or other glycoside conjugate, or may be an amino acid ester derivative.

Chemical Synthesis

Several methods for the chemical synthesis of macrolide (MC) compounds of the present invention are described herein. These and/or other well known methods may be modified and/or adapted in known ways in order to facilitate the synthesis of additional compounds within the scope of the present invention.

In one approach, compounds of type (7) are prepared as shown in the following scheme.

Compound (A) is isolated from the actinomycete mutant strain T658 as described in WO07110704 (wherein it is referred to as compound N831).

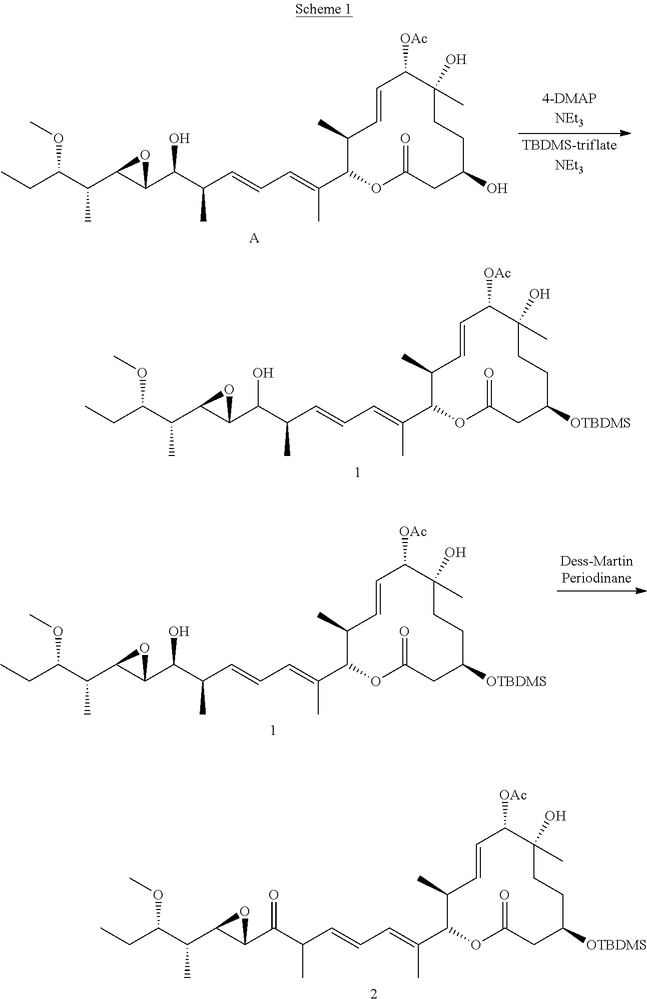

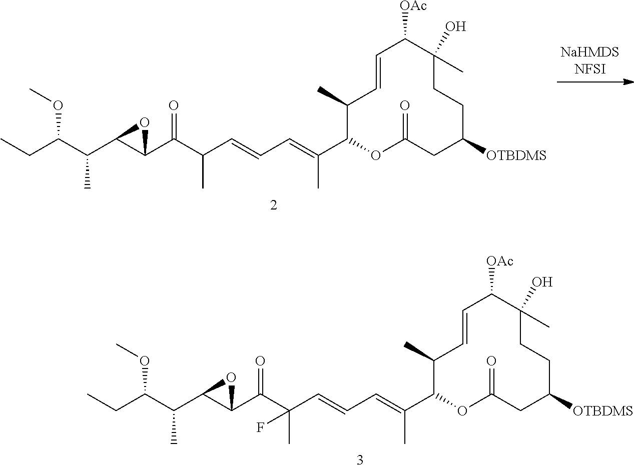

C3-TBDMS protected compounds (1) are prepared by treatment of precursor C17-OH macrolide (N831) with tert-butyldimethylsilyl trifluoromethanesulfonate in the presence of 4-Dimethylaminopyridine. C17-oxo compounds (2) are prepared by reaction of C3-protected C17-OH compound (1) with 1,1,1-Triacetoxy-1,1-dihydro-1,2-benziodoxol-3(1H)-one to oxidize the C17-OH, C16-F compounds (3) are prepared by reaction of (2) with N-Fluorodibenzenesulfonimide following addition of NaHDMS.

Reductive amination of C16-F/C17-oxo compounds (3) with either primary amine and Ti(OⁱPr)₄/NaBH₄ (Method A) or secondary amine (5) and sodium triacetoxyborohydride (Method B) to give C17-amine/C16-OH compounds (4) and (6) respectively.

Method A:

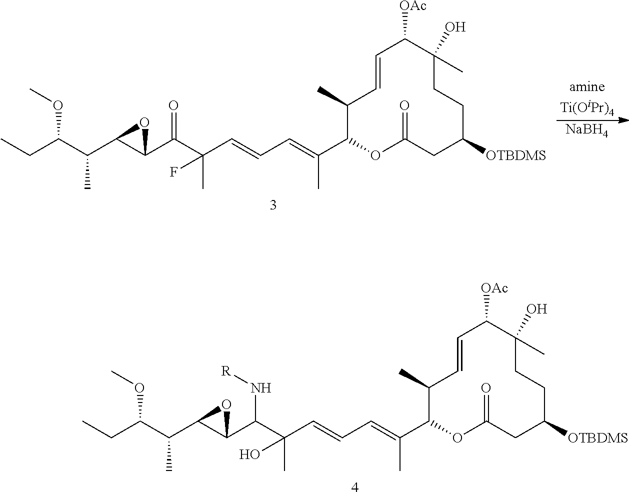

Method B:

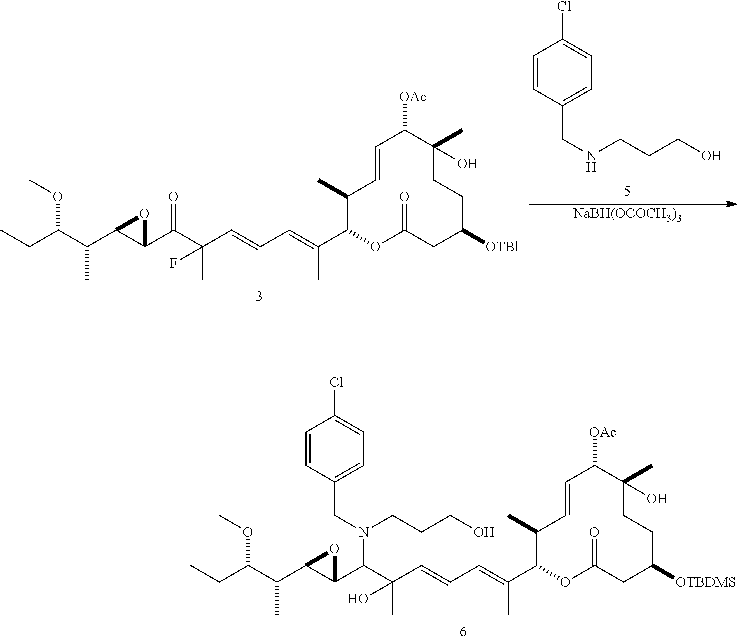

Deprotection of the C3 position is via TBAF to give target compounds (7).

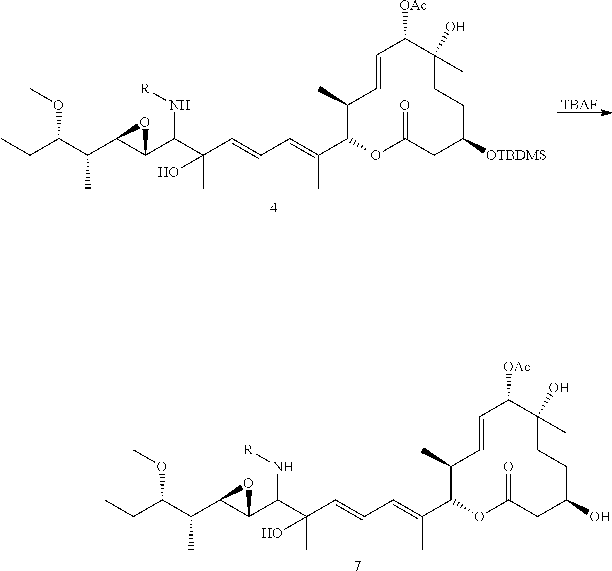

Oxazolidinone compounds are prepared from C-17-amino C-16 alcohol C-3TBDMS protected compounds (6) by reaction with triethylamine and triphosgene in dichloromethane at 0° C. Following reaction deprotection and work-up, the target oxazolidinone is isolated.

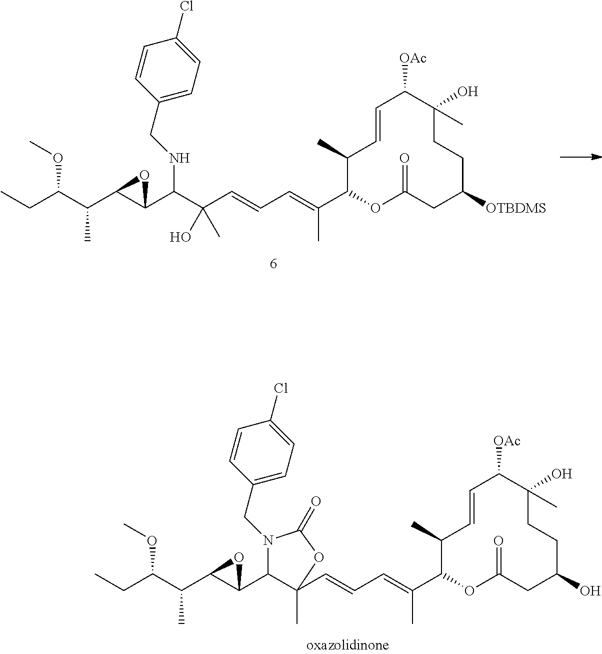

Compositions

One aspect of the present invention pertains to a composition (e.g., a pharmaceutical composition) comprising a MC compound, as described herein, and a pharmaceutically acceptable carrier, diluent, or excipient.

Another aspect of the present invention pertains to a method of preparing a composition (e.g., a pharmaceutical composition) comprising admixing a MC compound, as described herein, and a pharmaceutically acceptable carrier, diluent, or excipient.

Uses

The compounds described herein are useful, for example, in the treatment of proliferative conditions such as cancer, and diseases or conditions that are ameliorated by regulating (e.g., inhibiting) cell proliferation (e.g., proliferation of a cell) and/or promoting apoptosis.

Use in Methods of Inhibiting Cell Proliferation, Etc.

The MC compounds described herein, e.g., (a) regulate (e.g., inhibit) cell proliferation; (b) promote apoptosis; or (c) a combination of these.

One aspect of the present invention pertains to a method of regulating (e.g., inhibiting) cell proliferation (e.g., proliferation of a cell), promoting apoptosis, or a combination of one or more these, in vitro or in vivo, comprising contacting a cell with an effective amount of a MC compound, as described herein.

In one embodiment, the method is a method of regulating (e.g., inhibiting) cell proliferation (e.g., proliferation of a cell), in vitro or in vivo, comprising contacting a cell with an effective amount of a MC compound, as described herein.

In one embodiment, the method further comprises contacting the cell with one or more other agents selected from: (a) a DNA topoisomerase I or II inhibitor; (b) a DNA damaging agent; (c) an antimetabolite or TS inhibitor; (d) a microtubule targeted agent; and (e) ionising radiation.

In one embodiment, the method is performed in vitro.

In one embodiment, the method is performed in vivo.

In one embodiment, the MC compound is provided in the form of a pharmaceutically acceptable composition.

Any type of cell may be treated, including but not limited to, lung, gastrointestinal (including, e.g., bowel, colon), breast (mammary), ovarian, prostate, liver (hepatic), kidney (renal), bladder, pancreas, brain, and skin.

One of ordinary skill in the art is readily able to determine whether or not a candidate compound regulates (e.g., inhibits) cell proliferation, etc. For example, assays which may conveniently be used to assess the activity offered by a particular compound are described herein.

For example, a sample of cells (e.g., from a tumour) may be grown in vitro and a compound brought into contact with said cells, and the effect of the compound on those cells observed. As an example of “effect,” the morphological status of the cells (e.g., alive or dead, etc.) may be determined. Where the compound is found to exert an influence on the cells, this may be used as a prognostic or diagnostic marker of the efficacy of the compound in methods of treating a patient carrying cells of the same cellular type.

Use in Methods of Therapy

Another aspect of the present invention pertains to a MC compound, as described herein, for use in a method of treatment of the human or animal body by therapy.

In one embodiment, the method of treatment comprises treatment with both (i) a MC compound, as described herein, and (ii) one or more other agents selected from: (a) a DNA topoisomerase I or II inhibitor; (b) a DNA damaging agent; (c) an antimetabolite or TS inhibitor; (d) a microtubule targeted agent; and (e) ionising radiation.

Another aspect of the present invention pertains to (a) a DNA topoisomerase I or II inhibitor, (b) a DNA damaging agent, (c) an antimetabolite or TS inhibitor, or (d) a microtubule targeted agent, as described herein, for use in a method of treatment of the human or animal body by therapy, wherein the method of treatment comprises treatment with both (i) a MC compound, as described herein, and (a) the DNA topoisomerase I or II inhibitor, (b) the DNA damaging agent, (c) the antimetabolite or TS inhibitor, or (d) the microtubule targeted agent.

Use in the Manufacture of Medicaments

Another aspect of the present invention pertains to use of a MC compound, as described herein, in the manufacture of a medicament for use in treatment.

In one embodiment, the medicament comprises the MC compound.

In one embodiment, the treatment comprises treatment with both (i) a medicament comprising a MC compound, as described herein, and (ii) one or more other agents selected from: (a) a DNA topoisomerase I or II inhibitor; (b) a DNA damaging agent; (c) an antimetabolite or TS inhibitor; (d) a microtubule targeted agent; and (e) ionising radiation.

Another aspect of the present invention pertains to use of (a) a DNA topoisomerase I or II inhibitor, (b) a DNA damaging agent, (c) an antimetabolite or TS inhibitor, or (d) a microtubule targeted agent, as described herein, in the manufacture of a medicament for use in a treatment, wherein the treatment comprises treatment with both (i) a MC compound, as described herein, and (a) the DNA topoisomerase I or II inhibitor, (b) the DNA damaging agent, (c) the antimetabolite or TS inhibitor, or (d) the microtubule targeted agent.

Methods of Treatment

Another aspect of the present invention pertains to a method of treatment comprising administering to a patient in need of treatment a therapeutically effective amount of a MC compound, as described herein, preferably in the form of a pharmaceutical composition.

In one embodiment, the method further comprises administering to the subject one or more other agents selected from: (a) a DNA topoisomerase I or II inhibitor; (b) a DNA damaging agent; (c) an antimetabolite or TS inhibitor; (d) a microtubule targeted agent; and (e) ionising radiation.

Conditions Treated—Conditions Ameliorated by the Regulation of Cell Proliferation and/or Promoting Apoptosis

In one embodiment (e.g., of use in methods of therapy, of use in the manufacture of medicaments, of methods of treatment), the treatment is treatment of: a disease or condition that is ameliorated by regulating (e.g., inhibiting) cell proliferation (e.g., proliferation of a cell) and/or promoting apoptosis.

Conditions Treated—Proliferative Conditions and Cancer

In one embodiment (e.g., of use in methods of therapy, of use in the manufacture of medicaments, of methods of treatment), the treatment is treatment of: a proliferative condition.

The term “proliferative condition,” as used herein, pertains to an unwanted or uncontrolled cellular proliferation of excessive or abnormal cells which is undesired, such as, neoplastic or hyperplastic growth.

In one embodiment, the treatment is treatment of: a proliferative condition characterised by benign, pre-malignant, or malignant cellular proliferation, including but not limited to, neoplasms, hyperplasias, and tumours (e.g., histocytoma, glioma, astrocyoma, osteoma), cancers (see below), psoriasis, bone diseases, fibroproliferative disorders (e.g., of connective tissues), pulmonary fibrosis, atherosclerosis, smooth muscle cell proliferation in the blood vessels, such as stenosis or restenosis following angioplasty.

In one embodiment, the treatment is treatment of: cancer.

In one embodiment, the treatment is treatment of: lung cancer, small cell lung cancer, non-small cell lung cancer, gastrointestinal cancer, stomach cancer, bowel cancer, colon cancer, rectal cancer, colorectal cancer, thyroid cancer, breast cancer, ovarian cancer, endometrial cancer, prostate cancer, testicular cancer, liver cancer, kidney cancer, renal cell carcinoma, bladder cancer, pancreatic cancer, brain cancer, glioma, sarcoma, osteosarcoma, bone cancer, nasopharyngeal cancer (e.g., head cancer, neck cancer), skin cancer, squamous cancer, Kaposi's sarcoma, melanoma, malignant melanoma, lymphoma, or leukemia.

In one embodiment, the treatment is treatment of:
a carcinoma, for example a carcinoma of the bladder, breast, colon (e.g., colorectal carcinomas such as colon adenocarcinoma and colon adenoma), kidney, epidermal, liver, lung (e.g., adenocarcinoma, small cell lung cancer, and non-small cell lung carcinomas), oesophagus, gall bladder, ovary, pancreas (e.g., exocrine pancreatic carcinoma), stomach, cervix, thyroid, prostate, skin (e.g., squamous cell carcinoma);
a hematopoietic tumour of lymphoid lineage, for example leukemia, acute lymphocytic leukemia, B-cell lymphoma, T-cell lymphoma, Hodgkin's lymphoma, non-Hodgkin's lymphoma, hairy cell lymphoma, or Burkett's lymphoma;
a hematopoietic tumor of myeloid lineage, for example acute and chronic myelogenous leukemias, myelodysplastic syndrome, or promyelocytic leukemia;
a tumour of mesenchymal origin, for example fibrosarcoma or habdomyosarcoma;
a tumor of the central or peripheral nervous system, for example astrocytoma, neuroblastoma, glioma or schwannoma;
melanoma; seminoma; teratocarcinoma; osteosarcoma; xenoderoma pigmentoum; keratoctanthoma; thyroid follicular cancer; or Kaposi's sarcoma.

In one embodiment, the treatment is treatment of solid tumour cancer.

In one embodiment, the treatment is treatment of: lung cancer, breast cancer, ovarian cancer, colorectal cancer, melanoma, or glioma.

The anti-cancer effect may arise through one or more mechanisms, including but not limited to, the regulation of cell proliferation, the inhibition of cell cycle progression, the inhibition of angiogenesis (the formation of new blood vessels), the inhibition of metastasis (the spread of a tumour from its origin), the inhibition of invasion (the spread of tumour cells into neighbouring normal structures), or the promotion of apoptosis (programmed cell death). The compounds of the present invention may be used in the treatment of the cancers described herein, independent of the mechanisms discussed herein.

Treatment

The term “treatment,” as used herein in the context of treating a condition, pertains generally to treatment and therapy, whether of a human or an animal (e.g., in veterinary applications), in which some desired therapeutic effect is achieved, for example, the inhibition of the progress of the condition, and includes a reduction in the rate of progress, a halt in the rate of progress, alleviatiation of symptoms of the condition, amelioration of the condition, and cure of the condition. Treatment as a prophylactic measure (i.e., prophylaxis) is also included. For example, use with patients who have not yet developed the condition, but who are at risk of developing the condition, is encompassed by the term “treatment.”

For example, treatment includes the prophylaxis of cancer, reducing the incidence of cancer, alleviating the symptoms of cancer, etc.

The term “therapeutically-effective amount,” as used herein, pertains to that amount of a compound, or a material, composition or dosage form comprising a compound, which is effective for producing some desired therapeutic effect, commensurate with a reasonable benefit/risk ratio, when administered in accordance with a desired treatment regimen.

Combination Therapies

The term “treatment” includes combination treatments and therapies, in which two or more treatments or therapies are combined, for example, sequentially or simultaneously. For example, the compounds described herein may also be used in combination therapies, e.g., in conjunction with other agents, for example, cytotoxic agents, anticancer agents, etc. Examples of treatments and therapies include, but are not limited to, chemotherapy (the administration of active agents, including, e.g., drugs, antibodies (e.g., as in immunotherapy), prodrugs (e.g., as in photodynamic therapy, GDEPT, ADEPT, etc.); surgery; radiation therapy; photodynamic therapy; gene therapy; and controlled diets.

For example, it may be beneficial to combine treatment with a compound as described herein with one or more other (e.g., 1, 2, 3, 4) agents or therapies that regulates cell growth or survival or differentiation via a different mechanism, thus treating several characteristic features of cancer development.

One aspect of the present invention pertains to a MC compound as described herein, in combination with one or more additional therapeutic agents, as described below.

The particular combination would be at the discretion of the physician who would select dosages using his common general knowledge and dosing regimens known to a skilled practitioner.

The agents (i.e., the compound described herein, plus one or more other agents) may be administered simultaneously or sequentially, and may be administered in individually varying dose schedules and via different routes. For example, when administered sequentially, the agents can be administered at closely spaced intervals (e.g., over a period of 5-10 minutes) or at longer intervals (e.g., 1, 2, 3, 4 or more hours apart, or even longer periods apart where required), the precise dosage regimen being commensurate with the properties of the therapeutic agent(s).

The agents (i.e., the compound described here, plus one or more other agents) may be formulated together in a single dosage form, or alternatively, the individual agents may be formulated separately and presented together in the form of a kit, optionally with instructions for their use.

Combination Therapies Employing DNA Damaging Agents

As discussed herein, in some embodiments, the MC compound is employed in combination with (e.g., in conjunction with) one or more other agents selected from: (a) a DNA topoisomerase I or II inhibitor; (b) a DNA damaging agent; (c) an antimetabolite or TS inhibitor; (d) a microtubule targeted agent; and (e) ionising radiation.

When both a MC compound and one or more other agents are employed, they may be used (e.g., contacted, administered, etc.) in any order. Furthermore, they may be used (e.g., contacted, administered, etc.) together, as part of a single formulation, or separately, as separate formulations.

For example, in regard to methods of treatment employing both a MC compound and one or more other agents, treatment with (e.g., administration of) the MC compound may be prior to, concurrent with, or may follow, treatment with (e.g., administration of) the one or more other agents, or a combination thereof.

In one embodiment, treatment with (e.g., administration of) a MC compound is concurrent with, or follows, treatment with (e.g., administration of) the one or more other agents.

In one embodiment, the one or more other agents is a DNA topoisomerase I or II inhibitor; for example, Etoposide, Toptecan, Camptothecin, Irinotecan, SN-38, Doxorubicin, Daunorubicin.

In one embodiment, the one or more other agents is a DNA damaging agent; for example, alkylating agents, platinating agents, or compounds that generate free radicals; for example, Temozolomide, Cisplatin, Carboplatin, Mitomycin C, Cyclophosphamide, BCNU, CCNU, Bleomycin.

In one embodiment, the one or more other agents is an antimetabolite or TS inhibitor; for example, 5-fluorouracil, hydroxyurea, Gemcitabine, Arabinosylcytosine, Fludarabine, Tomudex, ZD9331.

In one embodiment, the one or more other agents is a microtubule targeted agent; for example, Paclitaxel, Docetaxel, Vincristine, Vinblastine.

In one embodiment, the one or more other agents is ionising radiation (e.g., as part of radiotherapy).

Other Uses

The MC compounds described herein may also be used as cell culture additives to regulate (e.g. inhibit) cell proliferation, etc.

The MC compounds described herein may also be used as part of an in vitro assay, for example, in order to determine whether a candidate host is likely to benefit from treatment with the compound in question.

The MC compounds described herein may also be used as a standard, for example, in an assay, in order to identify other compounds, other anti-proliferative agents, other anti-cancer agents, etc.

Kits

One aspect of the invention pertains to a kit comprising (a) a MC compound as described herein, or a composition comprising a MC compound as described herein, e.g., preferably provided in a suitable container and/or with suitable packaging; and (b) instructions for use, e.g., written instructions on how to administer the compound or composition.

In one embodiment, the kit further comprises one or more other agents selected from: (a) a DNA topoisomerase I or II inhibitor; (b) a DNA damaging agent; (c) an antimetabolite or TS inhibitor; and (d) a microtubule targeted agent.

The written instructions may also include a list of indications for which the active ingredient is a suitable treatment.

Routes of Administration

The MC compound or pharmaceutical composition comprising the MC compound may be administered to a subject by any convenient route of administration, whether systemically/peripherally or topically (i.e., at the site of desired action).

Routes of administration include, but are not limited to, oral (e.g., by ingestion); buccal; sublingual; transdermal (including, e.g., by a patch, plaster, etc.); transmucosal (including, e.g., by a patch, plaster, etc.); intranasal (e.g., by nasal spray); ocular (e.g., by eyedrops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir, for example, subcutaneously or intramuscularly.

The Subject/Patient

The subject/patient may be a chordate, a vertebrate, a mammal, a placental mammal, a marsupial (e.g., kangaroo, wombat), a rodent (e.g., a guinea pig, a hamster, a rat, a mouse), murine (e.g., a mouse), a lagomorph (e.g., a rabbit), avian (e.g., a bird), canine (e.g., a dog), feline (e.g., a cat), equine (e.g., a horse), porcine (e.g., a pig), ovine (e.g., a sheep), bovine (e.g., a cow), a primate, simian (e.g., a monkey or ape), a monkey (e.g., marmoset, baboon), an ape (e.g., gorilla, chimpanzee, orangutang, gibbon), or a human.

Furthermore, the subject/patient may be any of its forms of development, for example, a foetus.

In one preferred embodiment, the subject/patient is a human.

In one preferred embodiment, the subject/patient is not a human.

Formulations

While it is possible for the MC compound to be administered alone, it is preferable to present it as a pharmaceutical formulation (e.g., composition, preparation, medicament) comprising at least one MC compound, as described herein, together with one or more other pharmaceutically acceptable ingredients well known to those skilled in the art, including, but not limited to, pharmaceutically acceptable carriers, diluents, excipients, adjuvants, fillers, buffers, preservatives, anti-oxidants, lubricants, stabilisers, solubilisers, surfactants (e.g., wetting agents), masking agents, colouring agents, flavouring agents, and sweetening agents. The formulation may further comprise other active agents, for example, other therapeutic or prophylactic agents.

Thus, the present invention further provides pharmaceutical compositions, as defined above, and methods of making a pharmaceutical composition comprising admixing at least one MC compound, as described herein, together with one or more other pharmaceutically acceptable ingredients well known to those skilled in the art, e.g., carriers, diluents, excipients, etc. If formulated as discrete units (e.g., tablets, etc.), each unit contains a predetermined amount (dosage) of the compound.

The term “pharmaceutically acceptable,” as used herein, pertains to compounds, ingredients, materials, compositions, dosage forms, etc., which are, within the scope of sound medical judgment, suitable for use in contact with the tissues of the subject in question (e.g., human) without excessive toxicity, irritation, allergic response, or other problem or complication, commensurate with a reasonable benefit/risk ratio. Each carrier, diluent, excipient, etc. must also be “acceptable” in the sense of being compatible with the other ingredients of the formulation.

Suitable carriers, diluents, excipients, etc. can be found in standard pharmaceutical texts, for example, Remington's Pharmaceutical Sciences, 18th edition, Mack Publishing Company, Easton, Pa., 1990; and Handbook of Pharmaceutical Excipients, 5th edition, 2005.

The formulations may be prepared by any methods well known in the art of pharmacy. Such methods include the step of bringing into association the compound with a carrier which constitutes one or more accessory ingredients. In general, the formulations are prepared by uniformly and intimately bringing into association the compound with carriers (e.g., liquid carriers, finely divided solid carrier, etc.), and then shaping the product, if necessary.

The formulation may be prepared to provide for rapid or slow release; immediate, delayed, timed, or sustained release; or a combination thereof.

Formulations may suitably be in the form of liquids, solutions (e.g., aqueous, non-aqueous), suspensions (e.g., aqueous, non-aqueous), emulsions (e.g., oil-in-water, water-in-oil), elixirs, syrups, electuaries, mouthwashes, drops, tablets (including, e.g., coated tablets), granules, powders, losenges, pastilles, capsules (including, e.g., hard and soft gelatin capsules), cachets, pills, ampoules, boluses, suppositories, pessaries, tinctures, gels, pastes, ointments, creams, lotions, oils, foams, sprays, mists, or aerosols.

Formulations may suitably be provided as a patch, adhesive plaster, bandage, dressing, or the like which is impregnated with one or more compounds and optionally one or more other pharmaceutically acceptable ingredients, including, for example, penetration, permeation, and absorption enhancers. Formulations may also suitably be provided in the form of a depot or reservoir.

The compound may be dissolved in, suspended in, or admixed with one or more other pharmaceutically acceptable ingredients. The compound may be presented in a liposome or other microparticulate which is designed to target the compound, for example, to blood components or one or more organs.

Dosage

It will be appreciated by one of skill in the art that appropriate dosages of the MC compounds, and compositions comprising the MC compounds, can vary from patient to patient. Determining the optimal dosage will generally involve the balancing of the level of therapeutic benefit against any risk or deleterious side effects. The selected dosage level will depend on a variety of factors including, but not limited to, the activity of the particular MC compound, the route of administration, the time of administration, the rate of excretion of the MC compound, the duration of the treatment, other drugs, compounds, and/or materials used in combination, the severity of the condition, and the species, sex, age, weight, condition, general health, and prior medical history of the patient. The amount of MC compound and route of administration will ultimately be at the discretion of the physician, veterinarian, or clinician, although generally the dosage will be selected to achieve local concentrations at the site of action which achieve the desired effect without causing substantial harmful or deleterious side-effects.

Administration can be effected in one dose, continuously or intermittently (e.g., in divided doses at appropriate intervals) throughout the course of treatment. Methods of determining the most effective means and dosage of administration are well known to those of skill in the art and will vary with the formulation used for therapy, the purpose of the therapy, the target cell(s) being treated, and the subject being treated. Single or multiple administrations can be carried out with the dose level and pattern being selected by the treating physician, veterinarian, or clinician.

### Examples

The following examples are provided solely to illustrate the present invention and are not intended to limit the scope of the invention, as described herein.

Chemical Synthesis

General Methods and Materials

All reagents used in synthesis were commercial products and were used without further purification. Normal-phase flash chromatography was performed on a Teledyne Isco CombiFlash® Companion™ system with prepacked columns. Column chromatography was carried out on Merck silica gel 60 (230-400 mesh). Preparative thin-layer chromatography was performed using Merck silica gel 60 F254 20 cm×20 cm×0.25-1.0 mm precoated plates. Reverse-phase chromatography was carried out on a Varian ProStar HPLC system. Samples were purified on a XTerra® Prep RP18 OBD™ column (5 μm particle size, 19 mm×50 mm) using a gradient of 5% to 100% acetonitrile/water with 0.1% formic acid modifier at a flow rate of 15-18 ml/min. Analytical HPLC was performed on an Agilent 1100 Series HPLC system, peaks were acquired at UV 254 nm. The methods used a XTerra® MS C18 column (2.5 μm particle size, 4.6 mm×20 mm) using a linear gradient of 25-100% acetonitrile/water with 0.1% formic acid modifier at a flow rate of 1.5 mL/min over 7.5 minutes. High resolution mass spectra were generated on a Bruker microOTOF-Q instrument.

### Step 1: Synthesis of C3 O-TBDMS Compound (1)

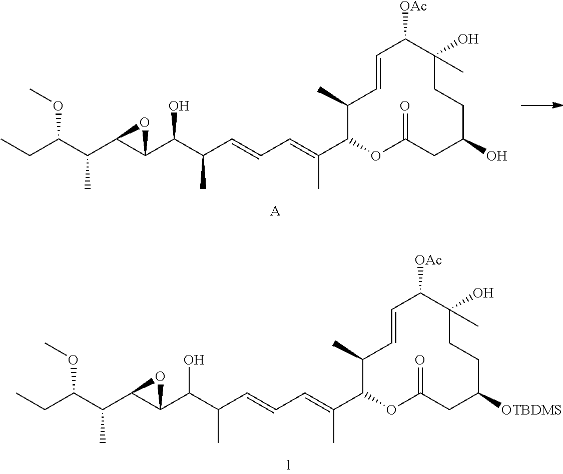

To a stirred solution of compound A (3.100 g, 5.477 mmol), 4-DMAP (670 mg, 5.477 mmol) and triethylamine (1.50 mL, 10.954 mmol) in dry dichloromethane (115 mL) at −78° C. was added drop-wise tert-butyldimethylsilyl trifluoromethanesulfonate (1.90 mL, 8.216 mmol) under an atmosphere of nitrogen gas. The resulting mixture was stirred at −78° C. for 1 hour. To the mixture was added a second portion of triethylamine (0.75 mL, 4.108 mmol) and tert-butyldimethylsilyl trifluoromethanesulfonate (0.95 mL, 4.108 mmol). The resulting mixture was stirred at −78° C. for 1 hour and then warmed to ambient temperature. To the mixture was added saturated aqueous sodium hydrogen carbonate (50 mL) and extracted into dichloromethane (50 mL×2). The combined organic extracts were washed with brine (50 mL), dried over sodium sulfate and concentrated under reduced pressure. The resulting residue was purified by silica gel column chromatography (hexane-ethyl acetate, 2:1) to give the desired compound C3-O-TBDMS compound (1) (1.33 g, 36%).

### Step 2: Synthesis of C-3 O-TBDMS C-17 oxo Compound (2)

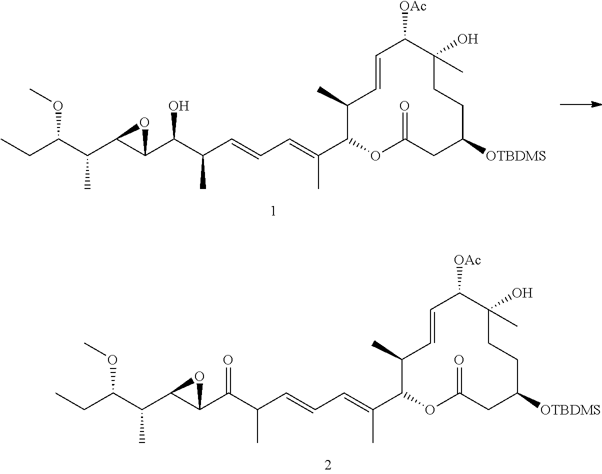

C3-O-TBDMS compound (1) (254 mg, 0.373 mmol) was dissolved in dry dichloromethane (20 mL) then cooled to 0° C. Dess-Martin Periodinane (396 mg, 0.932 mmol) was added portion-wise, and the reaction mixture was stirred at 0° C. overnight. After all starting material had reacted as indicated by TLC (silica gel: EtOAc/hexanes, 2:1); the reaction was worked-up by washing with saturated aqueous NaHCO₃. The aqueous layer was removed and extracted with dichloromethane. The combined organic layers were washed with brine, dried (Na₂SO₄), filtered and concentrated under reduced pressure. The crude product was purified by silica gel chromatography using CombiFlash (EtOAc/hexanes) to give the desired compound C3-O-TBDMS C-17oxo compound (2) (195 mg, 77%).

### Step 3: Synthesis of C-16F C-17oxo Compound (3)

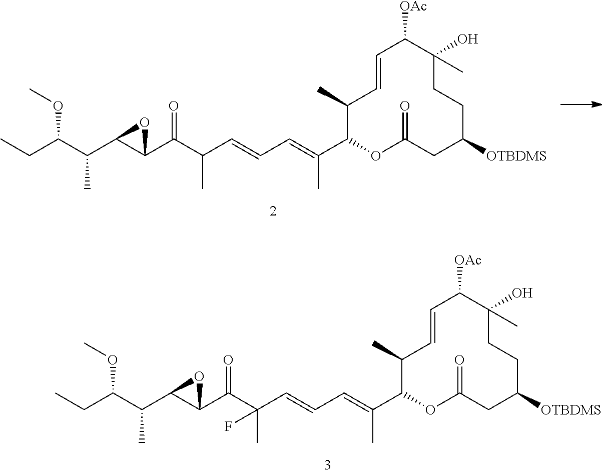

C3-O-TBDMS C-17oxo compound (2) (131 mg, 0.193 mmol) was dissolved in anhydrous THF (1.2 mL). It was cooled to −78° C. prior to the dropwise addition of NaHMDS (1.0 M in THF; 390 μL). N-Fluorodibenzenesulfonimide (122 mg, 0.387 mmol), dissolved in anhydrous THF, was then added. After all starting material had reacted, the reaction mixture was diluted with EtOAc and pH 7 phosphate buffer was added. The organic layer was separated, dried (Na₂SO₄), filtered and concentrated under reduced pressure. The crude product was purified by silica gel chromatography using CombiFlash (EtOAc/hexanes) to give the fluorinated product C-16F C-17oxo compound (3) (84 mg, 63%).

### Step 4: Reductive Amination of C-16F C-17oxo Compound (3)

Method a (General Procedure): with Ti(OiPr)₄

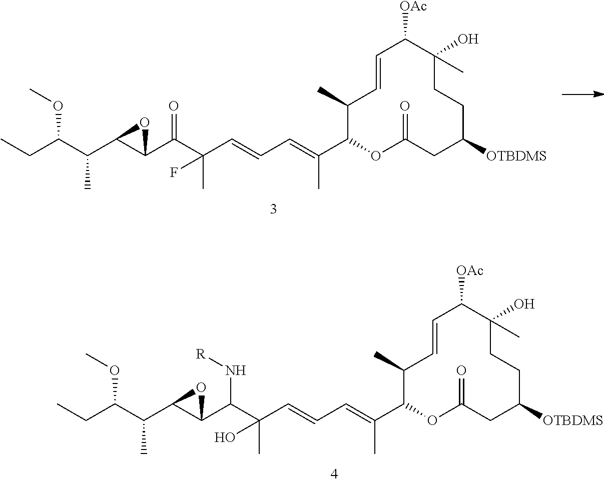

To C-16F C-17oxo compound (3) (0.03 mmol scale) was added 0.9 eq. of a primary amine dissolved in dry dichloromethane (0.10 mL). To this was added 4 eq. Ti(OiPr)₄ and the solution was stirred at 0° C. for 3 hours. 300 μL MeOH was then added slowly and spatula tip (˜2 eq.) sodium borohydride and stirred at 0° C. for 20 minutes. It was then quenched with a few drops distilled water and to the white precipitate is added ethyl acetate and stirring continued for 20 minutes at 0° C. The precipitate formed was filtered and washed several times with fresh ethyl acetate and a final wash of dichloromethane and the organic layers are then combined and purified by reverse-phase HPLC eluting with a gradient of acetonitrile in water (0.1% formic acid modifier) to yield 4 in approx. 25% yield as a mixture of diastereomers.

Method B: with Sodium Triacetoxyborohydride

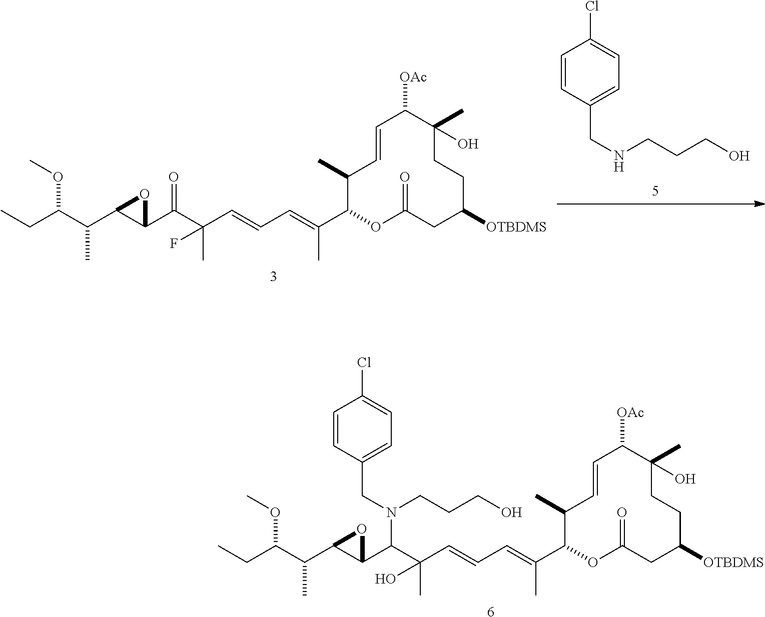

A solution of compound 3 (82.9 mg, 0.119 mmol), 5 (238 mg, 1.19 mmol), and acetic acid (0.070 mL, 1.22 mmol) in dry 1,2-dichlororthane (3.0 mL) was stirred at ambient temperature for 3 hours under an atmosphere of nitrogen. To the mixture was added sodium triacetoxyborohydride (255 mg, 1.20 mmol). The reaction mixture was stirred at ambient temperature for 19 hours. To the mixture was added saturated aqueous sodium hydrogen carbonate (30 mL). The mixture was extracted with dichloromethane (30 mL, 20 mL×2). The combined organic extracts were washed with brine (20 mL), dried over sodium sulfate, and concentrated under reduced pressure. The resulting residue was purified by preparative thin layer chromatography (silica gel, 200 mm×200 mm×1.0 mm×4 plates, hexane-ethyl acetate-methanol (10:10:1) to give 6 (8.8 mg, 8.4%).

### Step 5: Deprotection of Product Amino Alcohols

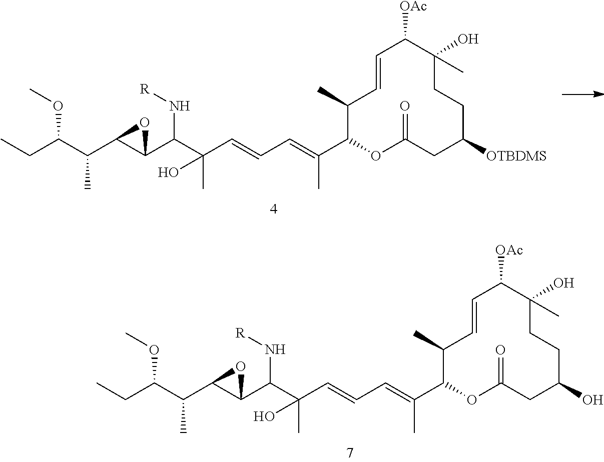

To the purified amino alcohols 4 obtained above were dissolved in THF and 10 eq. tetra-n-butylammonium fluoride (TBAF) (1.0 M in THF) was added and stirred at room temperature for 2-8 hours or till deprotection was complete (vide HPLC). The deprotected products were then purified by reverse-phase HPLC.

See Table below for analytical data on synthesized compounds.

Alternately, the fractions of 4 above were allowed to stand at room temperature in the HPLC solvent and the TBDMS deprotection takes place due to the acidity of the HPLC solvents, i.e. 0.1% formic acid. On complete deprotection, the solvents are removed in vacuo and the resulting residue is lyophilized for 12 hours from a solution in 1:1 acetonitrile-water.

Synthesis of C17-N oxazolidinone MC-012a, MC-012b:

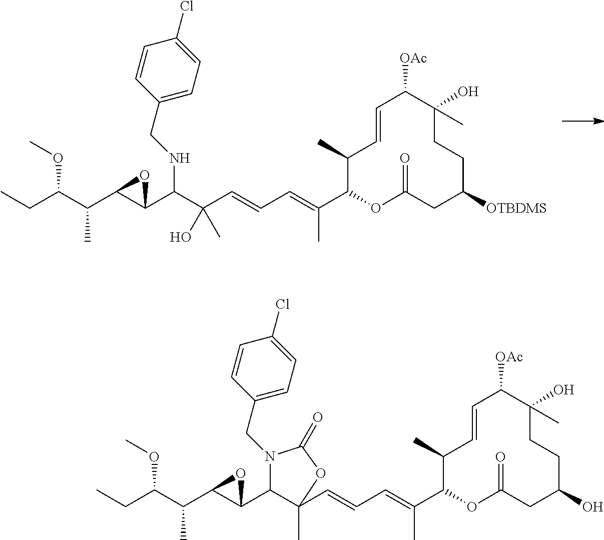

To 9.6 mg (0.012 mm) of amino alcohol dissolved in dichloromethane was cooled and to 0° C. and to it was added triethylamine 65 μL (0.45 mm, 45 eq.) and 17 mg (0.05 mm, 5 eq.) of triphosgene and the resulting mixture stirred for 15 minutes. The reaction was worked up by the slow addition of water and EtOAc at 0° C. and the organic layer separated and purified by reverse phase HPLC to yield 2 fractions that were deprotected as usual to yield MC-012a (1 mg) and MC-012b (0.8 mg) as the 2 diastereomers of the oxazolidinone.

Synthesis of C16-C17-diamino Compounds

This was done as in Step 4 Method A above but using >1 eq of amine in the reductive amination step.

The piperazine analog MC-003 was made by using a bis amine (ethylene diamine) in the reductive amination step. The homopiperazine analogue was made using 1,3-diaminopropane in the reductive amination step.

Synthesis of Building Block Amines

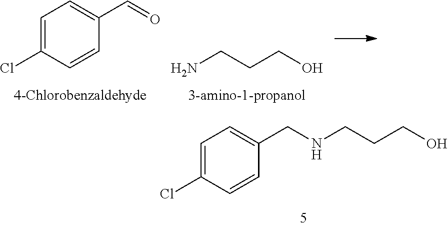

To a stirred solution of 4-chlorobenzaldehyde (422 mg, 3.00 mmol) in methanol (30.0 mL) at ambient temperature was added 3-amino-1-propanol (230 μL, 3.00 mmol) under an atmosphere of nitrogen gas. The resulting mixture was stirred at ambient temperature for 3 hours. To the mixture was added sodium borohydride (182 mg, 4.80 mmol) and stirred at ambient temperature for 1 hour. The mixture was concentrated under reduced pressure. To the mixture was added saturated sodium hydrogen carbonate (30 mL) and extracted with dichloromethane (30 mL, 20 mL×2). The combined organic extracts were washed with brine (20 mL), dried over sodium sulfate and concentrated under reduced pressure. The resulting residue was purified by silica gel column chromatography (dichloromethane-methanol (6:1)) to give 5 (381 mg, 64%).
Reference: J. Org. Chem. 1996, 61, 3849-3862.

In a similar manner, the following amines were synthesized starting from the appropriate aldehyde and primary amines, with the product structure being confirmed by mass spec and NMR data:

| | Mass |
|---|---|
| | Observed |
| Amine Structure | (M + 1) |
| 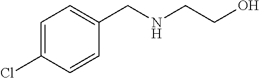 | 186.1 |
| 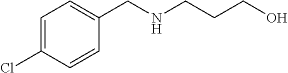 | 200.1 |
| 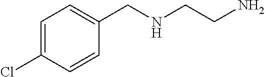 | 185.1 |
| 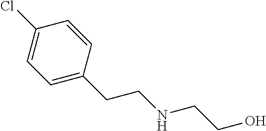 | 200.1 |
| 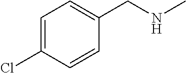 | 156.1 |
| 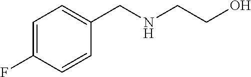 | 170.1 |
| 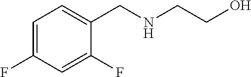 | 188.1 |
| 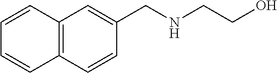 | 202.1 |
| 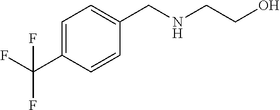 | 220.1 |
| 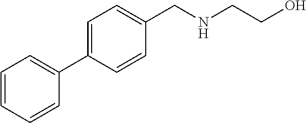 | 228.1 |
| 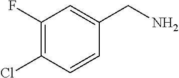 | 160.0 |
| 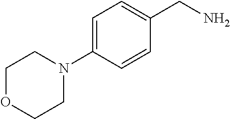 | 193.1 |
| 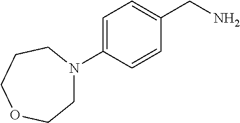 | 207.2 |
| 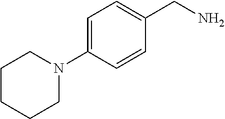 | 191.2 |
| 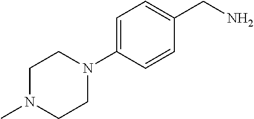 | |
| 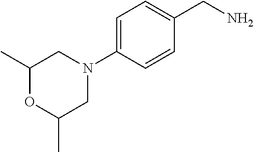 | 221.2 |
| 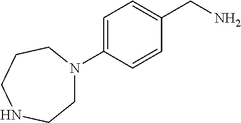 | 206.2 |
| 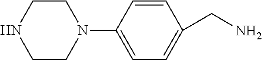 | 192.1 |
| 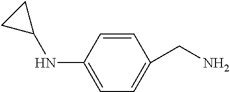 | 146.1 |
| 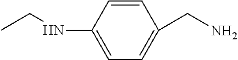 | 134.1 |

### Representative Example

### Synthesis of (4-(1,4-oxazepan-4-yl)phenyl)methanamine

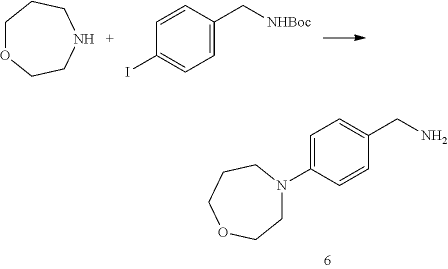 [1613] Reference: JACS 2006, 128, 8742-8743 A. Shafir, S. L. Buchwald and references cited therein.

### Step 1: Boc Protection of 4-iodobenzylamine

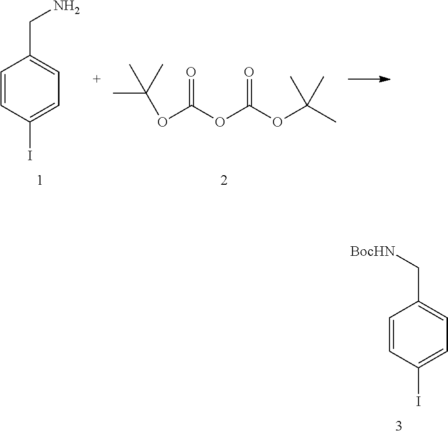

To a stirred solution of 4-Iodobenzylamine 1 (0.4 g, 1.716 mmol), 4-DMAP (catalytic amount) and triethylamine (241 μL, 1.716 mmol) in THF (8 mL), Boc anhydride (455 mg, 2.059 mmol) was added at ambient temperature. The resulting mixture was stirred at 40-50° C. for 2 hours or till protection was complete. The mixture was concentrated under reduced pressure. To the mixture was added water (20 mL) and extracted with ethyl acetate (20 mL, 10 mL×2). The combined organic extracts were dried over sodium sulfate and concentrate under reduced pressure to give the desired product 3 (685 mg).

### Step 2: Buchwald Chemistry

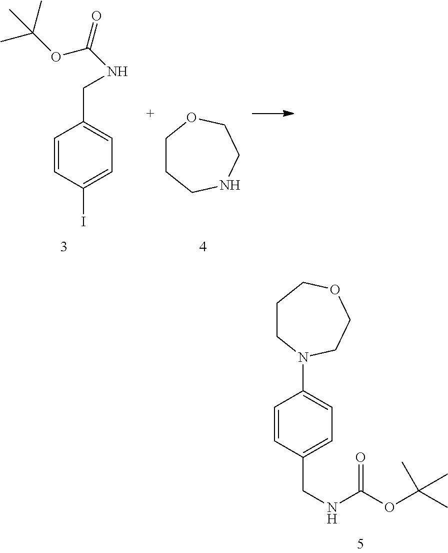

To the Boc-protected amine 3 (138.9 mg, 0.4169 mmol) obtained above, Copper iodide (7.94 mg, 0.04169 mmol) and cesium carbonate (271.8 mg, 0.8338 mmol) were added under an atmosphere of nitrogen. Resulting mixture was dissolved in DMF (0.5 mL) and homomorpholine 4 (86 mg, 0.625 mmol) and 2-acetylcyclohexanone (22 μL, 0.167 mmol) were added at ambient temperature. The resulting mixture was stirred at 80° C. overnight or till protection was complete. To the mixture was added water (20 mL) and extracted with ethyl acetate (20 mL, 10 mL×2). The combined organic extracts were dried over sodium sulfate and concentrate under reduced pressure. The resulting residue was purified by reverse-phase HPLC eluting a gradient of acetonitrile in water (0.1% formic acid modifier) to yield 5.

### Step 3: Deprotection of benzylamines

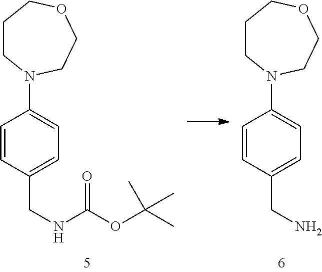

To the purified Boc amines 5 obtained above was dissolved in 1:1 TFA/DCM (Trifluoroacetic acid/dichloromethane) and stirred at ambient temperature for 30 mins. The mixture was concentrated under reduced pressure and purified by reverse-phase HPLC eluting a gradient of acetonitrile in water (0.1% formic acid modifier) to yield 6.

### Representative Examples

### Synthesis of MC-057a, MC-057b, MC-058a and MC-058b

(i) Synthesis of C-17-Amino-C-16-Fluoro Compound

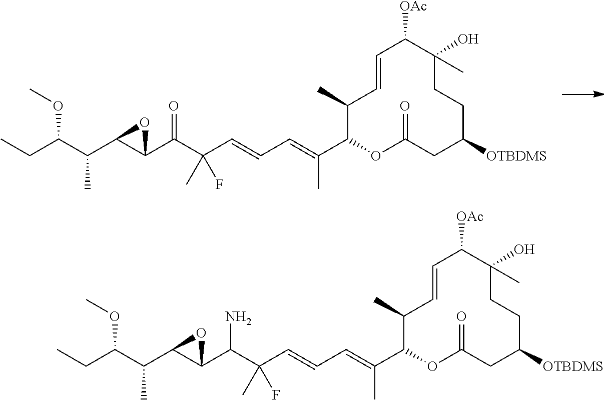

The C-16-fluoro-C-17-oxo compound (30.5 mg, 0.0438 mmol) and NH₄Cl (14.0 mg, 0.262 mmol) were dissolved in absolute EtOH (0.30 mL). Et₃N (22 μL, 0.218 mmol) was added and after stirring at room temperature for 5 min, Ti(i-OPr)₄ (50 μL, 0.176 mmol) was added dropwise. After stirring at room temperature for 16 hr, NaBH₄ (7.2 mg, 0.189 mmol) was added. When the reaction was complete, the reaction mixture was quenched with a few drops of deionised H₂O. It was then stirred for 1-2 hr, extracted with EtOAc, dried (Na₂SO₄), filtered and concentrated. The crude product (40.5 mg) was used for the next step without further purification or characterization. m/z 698.4464 [M+H]⁺.

(ii) Synthesis of C-17-Amino-C-16-Hydroxy Compound

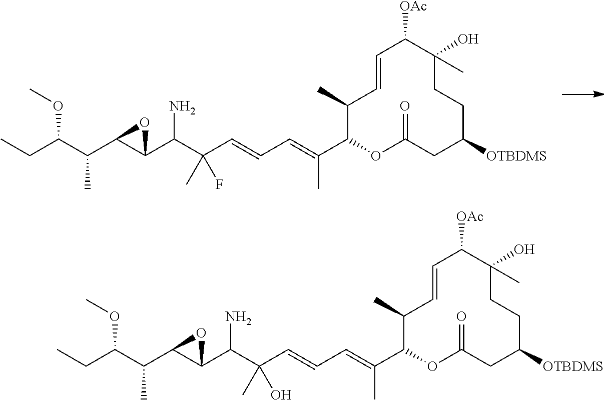

The crude C-17-amino-C-16-fluoro compounds (40.3 mg) obtained from the previous step was dissolved in MeCN/H₂O (0.50 mL, 3:2). The reaction mixture was stirred at room temperature for 24 hr. After all starting material had reacted, solvents were removed under reduced pressure. The crude product (40.0 mg) was used as such for the next step. m/z 696.4465 [M+H]⁺.

(iii) Synthesis of C-17-Sulfonamide-C-16-Hydroxy Compound

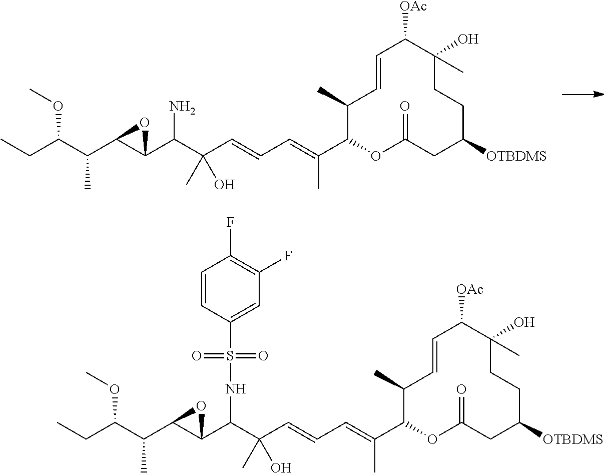

The C-17-amino-C-16-hydroxy compound (11.8 mg, 0.017 mmol) was dissolved in anhydrous CH₂Cl₂ (0.50 mL). Et₃N (20 μL, 11.7 mmol) was added followed by 3,4-di-fluoro-benzenesulfonyl chloride (10 μl, 0.0743 mmol). The reaction mixture was stirred at room temperature and after all starting material had reacted, it was quenched by adding deionised H₂O (2 mL) and diluting with EtOAc (5 mL). The aqueous layer was separated and extracted with EtOAc. Combined organic layers was successively washed with sat. aq. NaHCO₃ and brine; dried (Na₂SO₄), filtered and concentrated. Crude material was purified by silica gel chromatography using CombiFlash (EtOAc/hexanes) to give the desired product (2.7 mg, 18%).

(iv) C-3-TBDMS Deprotection

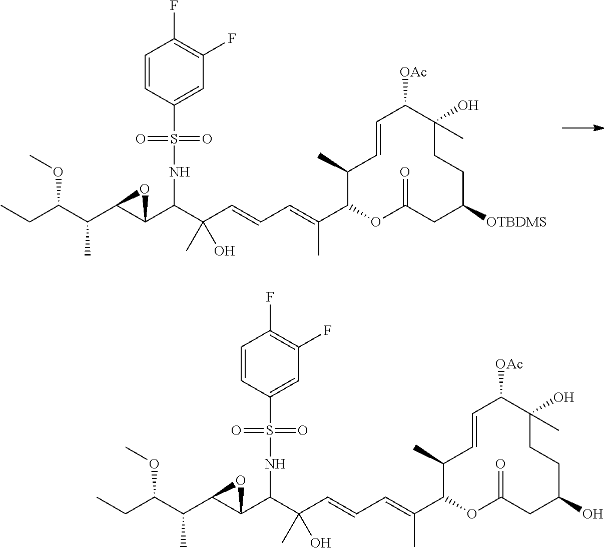

The C-17-sulfonamide-C-16-hydroxy compound (5.4 mg, 0.0062 mmol) was dissolved in anhydrous THF (1 mL). TBAF (50 μL, 0.05 mmol) was added and the reaction mixture stirred at room temperature. After all starting material had reacted, the solvent was removed under reduced pressure and the crude product was purified by preparative RP-HPLC to give MC-058a (2.0 mg, 41%) and MC-058b (1.9 mg, 39%). m/z 780.3236 [M+H]⁺; 2.998 min (25-100% ACN/H₂O, +0.1% FA) and m/z 780.3245 [M+H]⁺; 2.990 min (25-100% ACN/H₂O, +0.1% FA), respectively.

(v) Synthesis of C-17-Benzamide-C16-Fluoro Compound

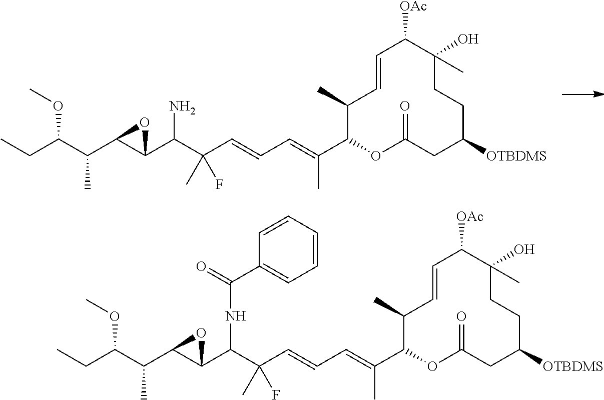

Crude C17-amino-C₁₋₆-fluoro compound (25.0 mg) was dissolved in anhydrous CH₂Cl₂ (1 mL). Pyridine (50 μL, 0.633 mmol) was added followed by benzoyl chloride (40 μl, 0.285 mmol). The reaction mixture was stirred at room temperature. After all starting material had reacted, it was quenched by adding deionised H₂O (2 mL) and diluting with EtOAc (5 mL). The aqueous layer was separated and extracted with EtOAc. Combined organic layers was successively washed with sat. aq. NaHCO₃ and brine; dried (Na₂SO₄), filtered and concentrated to give the crude product (31.4 mg).

(vi) C-3-TBDMS Deprotection

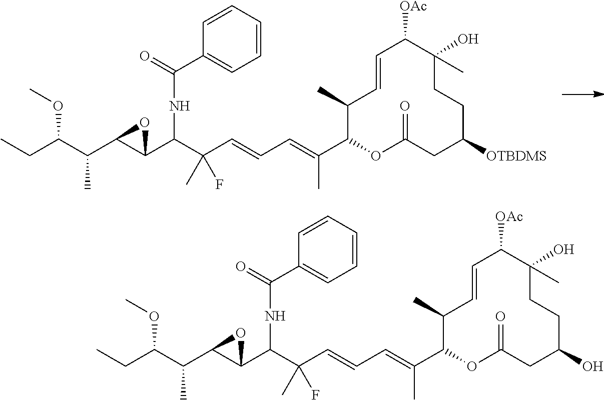

The crude C-17-benzamide-C-16-fluoro compound (31.4 mg) obtained from the previous step was dissolved in anhydrous THF (1 mL). TBAF (0.40 mL, 0.40 mmol) was added and the reaction mixture stirred at room temperature. After all starting material had reacted, the solvent was removed under reduced pressure and the crude product was purified by preparative RP-HPLC to give the desired product (5.5 mg, 22% over 2 steps). m/z 688.3928 [M+H]⁺.

(vii) Synthesis of C-17-Benzamide-C16-Hydroxy Compound

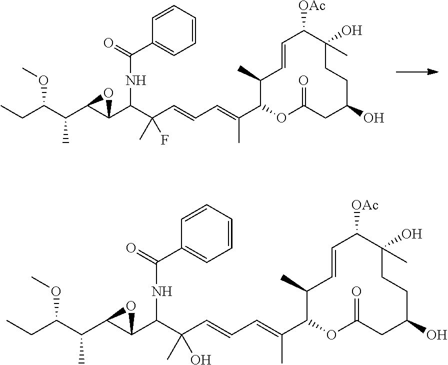

The C-17-benzamide-C₁₋₆-fluoro compound (5.5 mg, 0.0080 mmol) was dissolved in MeCN/H₂O (0.40 mL, 1:1). Formic acid (50 μL) was added and the reaction mixture stirred at room temperature. After all starting material had reacted, solvents were removed under reduced pressure. Crude material was purified by preparative RP-HPLC to give MC-057a (0.9 mg, 16%) and MC-057b (1.0 mg, 18%). m/z 686.3800 [M+H]⁺; 1.677 min (25-100% ACN/H₂O, +0.1% FA) and m/z 686.3778 [M+H]⁺; 1.834 min (25-100% ACN/H₂O, +0.1% FA), respectively.

Analytical Data

The names of the compounds in the table below reflect whether or not more than one diastereomer of the compound was isolated; for those compounds where more than one diastereomer was isolated, a suffix “a”, “b”, etc is provided for each diastereomer. For those compounds where more than one diastereomer has been isolated, reference herein to such compounds without the suffix is a reference to the compound in any one of its diastereomeric forms and/or a reference to a mixture of such diastereomers.

Note that some of the compounds are shown with an associated solvent molecule. Naturally, reference herein to a compound, e.g. MC-001, is intended to be a reference to that compound independently of whether or not there is an associated solvent molecule (further, the reference herein to solvates of such compounds includes the compounds with such solvent molecules).

| | | Mass | HPLC | |
|---|---|---|---|---|
| | | Observed | retention | HPLC |
| Compound | Product Structure | (M + 1) | time | method |
| MC-001 | 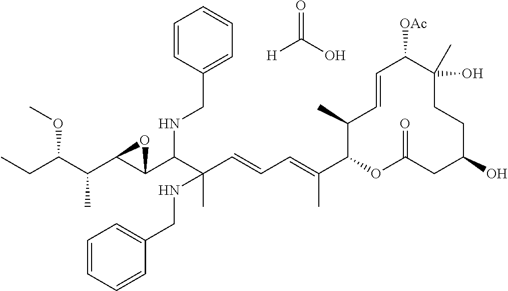 | 761.6 | 2.344 | 25- 100B2AC 110 min |
| MC-003 | 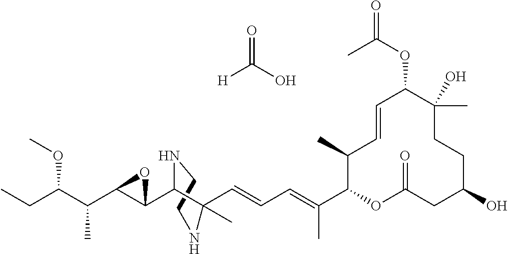 | 607.4 | 3.041 | 25- 100B2AC 110 min |
| MC-004a MC-004b | 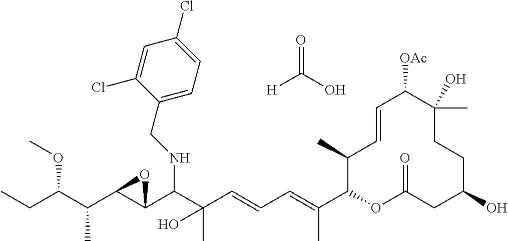 | 740.3 740.3 | 1.806 1.591 | 25- 100B2AC 110 min |
| MC-005 | 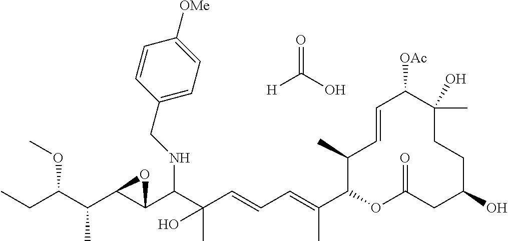 | 702.4 | 1.254-70.9%, 1.435-24.9% | 25- 100B2AC 110 min |
| MC-006a MC-006b MC-006c | 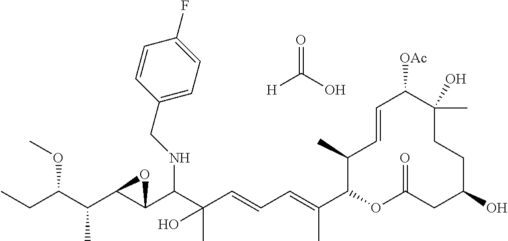 | 690.4 690.4 690.4 | 1.399 1.414 1.268 | 25- 100B2AC 110 min |
| MC-007a MC-007b MC-007c MC-007d | 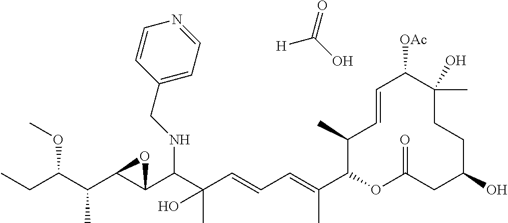 | 673.4 673.4 673.4 | 1.922-51.4%, 1.803-43% 2.012-36.5%, 2.118-31.7% 2.06-75.6%, 2.001-24.3% 1.990 | 10- 100B2AC 110 min |
| MC-008a MC-008b MC-008c | 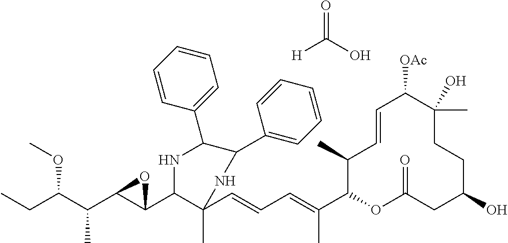 | 759.5 759.5 759.5 | 2.600 2.214 2.568 | 25- 100B2AC 110 min |
| MC-009a MC-009b MC-009c MC-009d MC-009e | 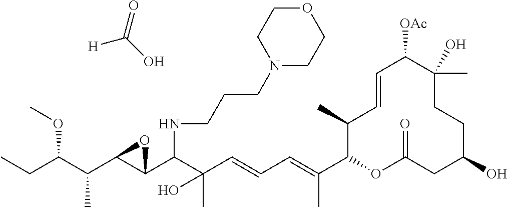 | 709.5 709.5 709.5 709.5 709.5 | 1.811 1.875 1.920 1.943 1.759 | 10- 100B2AC 110 min |
| MC-010a MC-010b | 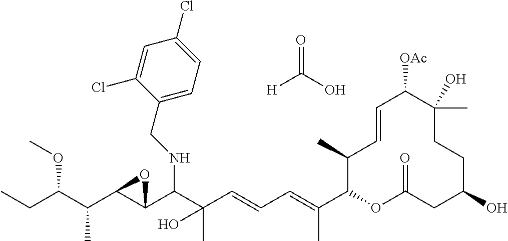 | 740.3 740.3 | 1.622 1.811 | 25- 100B2AC 110 min |
| MC-011 | 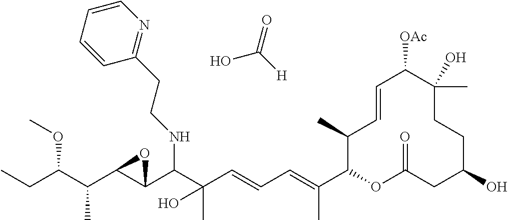 | | 2.369 | 10- 100B2AC 110 min |
| MC-012a MC-012b | 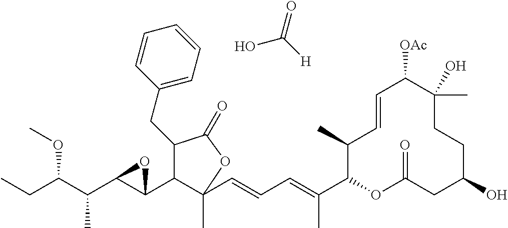 | | 3.568-78% 3.662-22% 3.655 | |
| MC-013 | 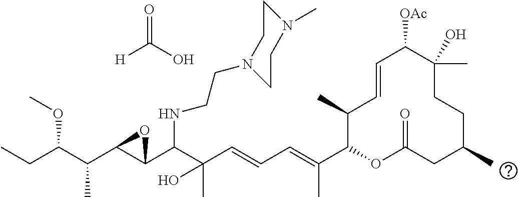 | 708.5 | 1.848 | 10- 100B2AC 110 min |
| MC-014a MC-014b MC-014c | 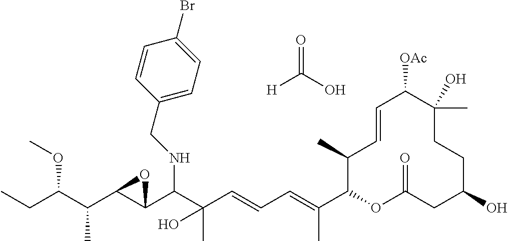 | 750.3 750.3 750.3 | 1.630 1.571 1.433 | 25- 100B2AC 110 min |
| MC-015a MC-015b | 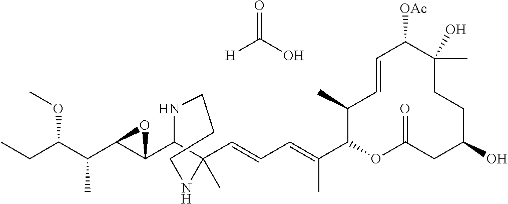 | 665.4 665.4 | 2.228 1.964 | 10- 100B2AC 110 min |
| MC-016a MC-016b | 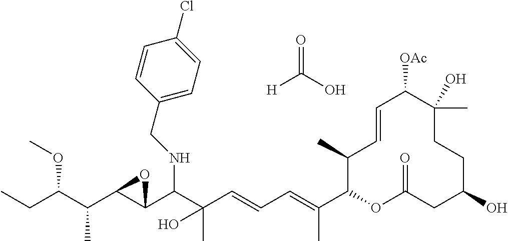 | 706.4 706.4 | 1.342 1.363 | 25- 100B2AC 110 min |
| MC-017a MC-017b | 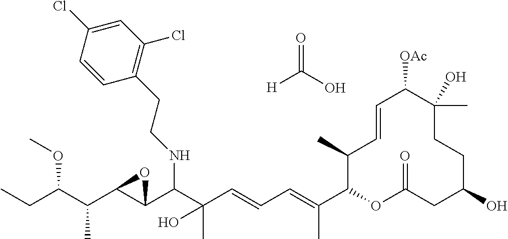 | 754.3 754.3 | 2.020 1.885 | 25- 100B2AC 110 min |
| MC-018a MC-018b MC-018c | 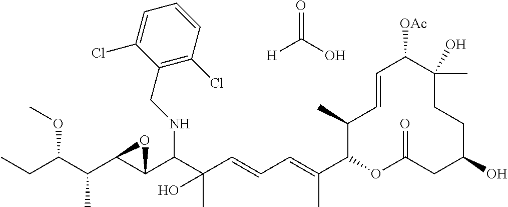 | 740.3 740.3 740.3 | 1.624 1.380 1.509 | 25- 100B2AC 110 min |
| MC-019a MC-019b MC-019c | 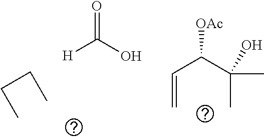 | 636.4 636.4 636.4 | 2.363 2.469 2.394 | 10- 100B2AC 110 min |
| MC-020a MC-020b | 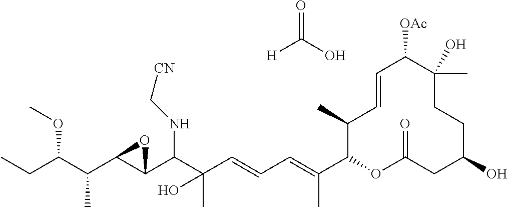 | 643.4 643.4 | 1.280 1.242 | 25- 100B2AC 110 min |
| MC-021 | 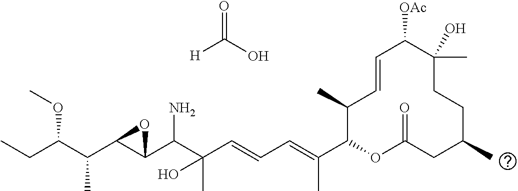 | 582 | 3.284-51.2% 3.421-27% | 0-50- 100B2AC 110 min |
| MC-022 | 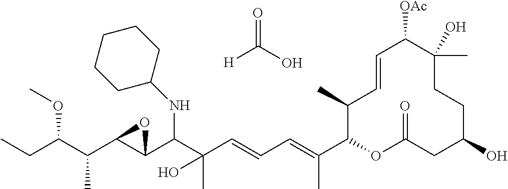 | 664.4 | 2.651 | 10- 100B2AC 110 min |
| MC-023a MC-023b | 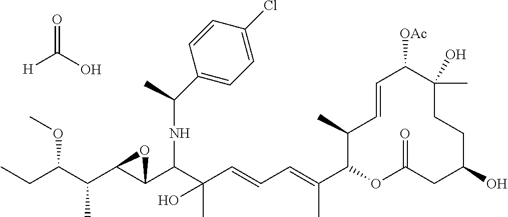 | 720.4 720.4 | 1.857 1.650 | 25- 100B2AC 110 min |
| MC-024a MC-024b MC-024c | 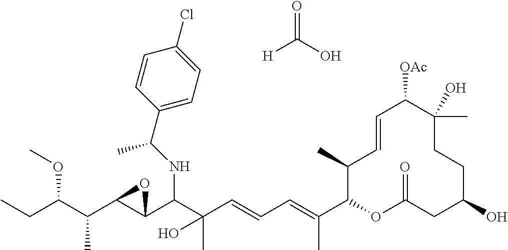 | 720.4 720.4 720.4 | 1.797 1.632-49.6% 1.822-49% 1.822 | 25- 100B2AC 110 min |
| MC-025a MC-025b | 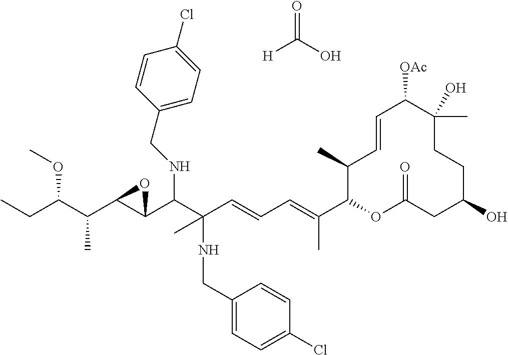 | 829.4 829.4 | 2.807 3.015 | 25- 100B2AC 110 min |
| MC-026a MC-026b MC-026c MC-026d | 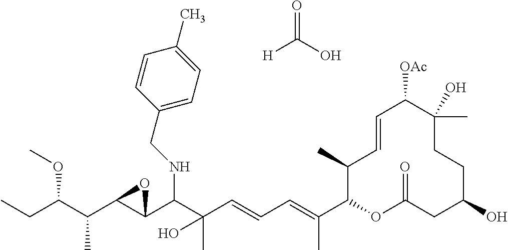 | 686.4 686.4 686.4 686.4 | 1.449 1.494 1.371-77.6% 1.551-19.5% 1.543 | 25- 100B2AC 110 min |
| MC-027a MC-027b | 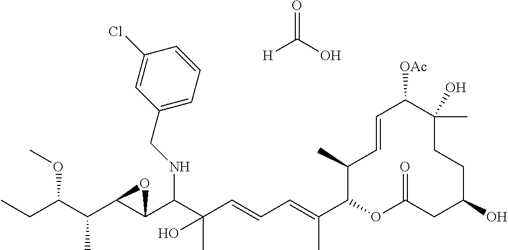 | 706.4 706.4 | 1.505 1.396-60% 1.567-40% | 25- 100B2AC 110 min |
| MC-028a MC-028b | 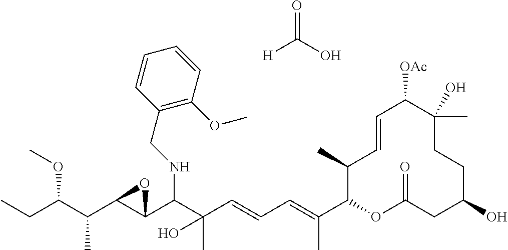 | 702.4 702.4 | 1.469 1.530 | 25- 100B2AC 110 min |
| MC-029 | 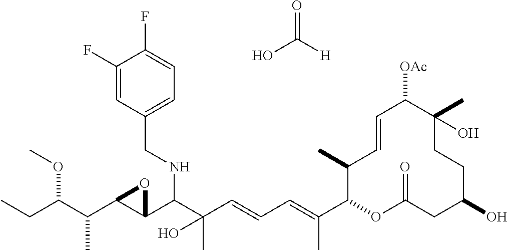 | 708.4 | 1.26 | 25- 100B2AC 110 min |
| MC-030a MC-030b | 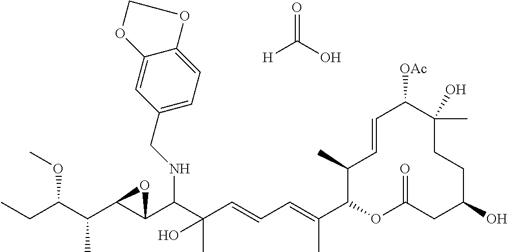 | 716.4 716.4 | 1.197-53.2% 1.345-46.7% 1.311 | 25- 100B2AC 110 min |
| MC-031 | 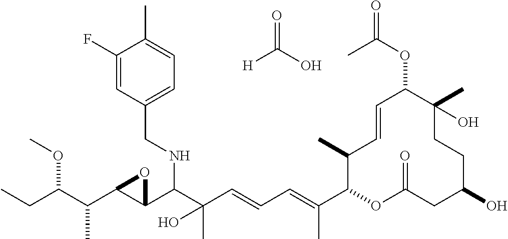 | 704.4 | 1.32 | 25- 100B2AC 110 min |
| MC-032 | 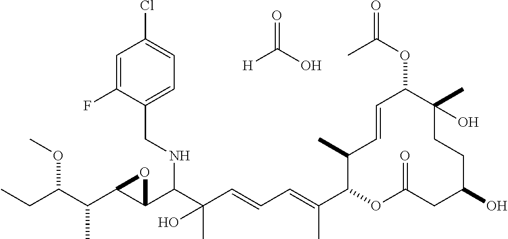 | 724.4 | 1.37 | 25- 100B2AC 110 min |
| MC-033 | 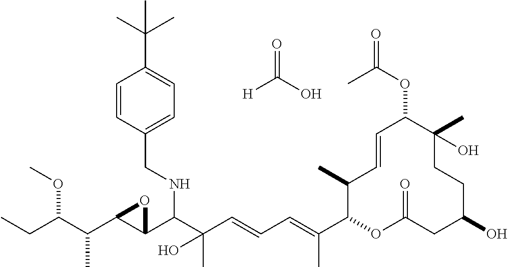 | 728.5 | 2.07 | 25- 100B2AC 110 min |
| MC-034 | 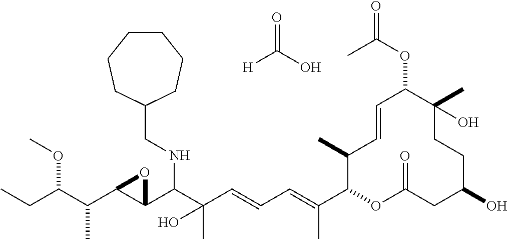 | 692.5 | 1.75 | 25- 100B2AC 110 min |
| MC-035 | 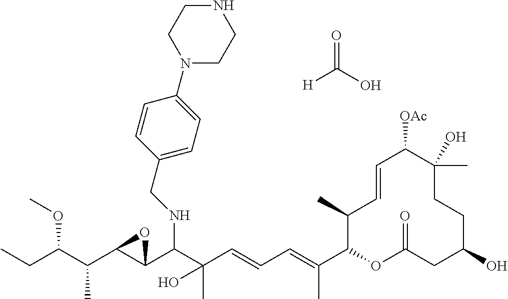 | 756.5 | 3.178-62.4% 3.276-34% | 0-50- 100B2AC 110 min |
| MC-036 | 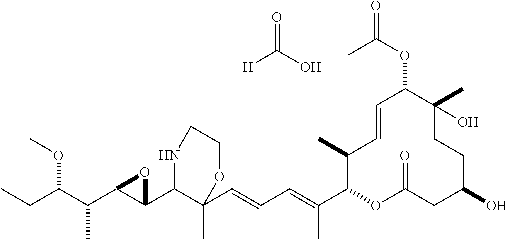 | 608.4 | 1.26 | 25- 100B2AC 110 min |
| MC-037a MC-037b MC-037c MC-037d | 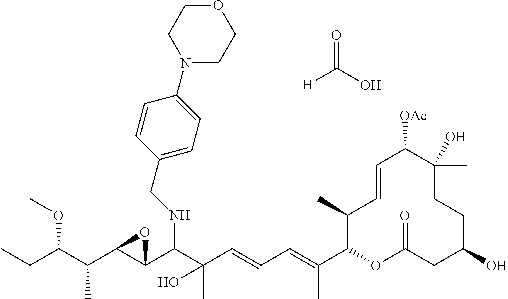 | 756.5 756.5 756.5 756.5 | 2.048 2.063 3.718-34.7% 3.820-63.8% 3.825 | 0-50- 100B2AC 110 min |
| MC-038 | 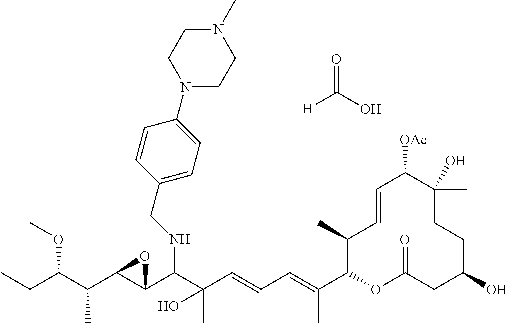 | 770.5 | 3.164 | 0-50- 100B2AC 110 min |
| MC-039 | 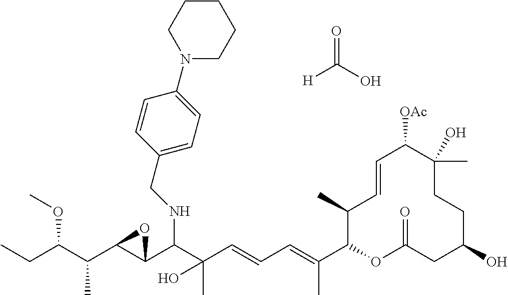 | | 0.820 | 25- 100B2AC 110 min |
| MC-040 | 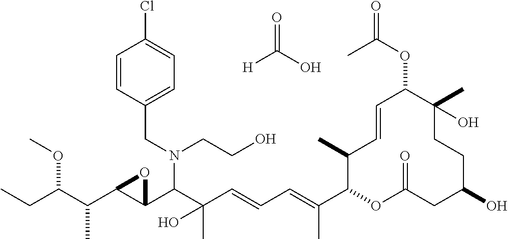 | 750.4 | 1.67 | 25- 100B2AC 110 min |
| MC-041a MC-041b | 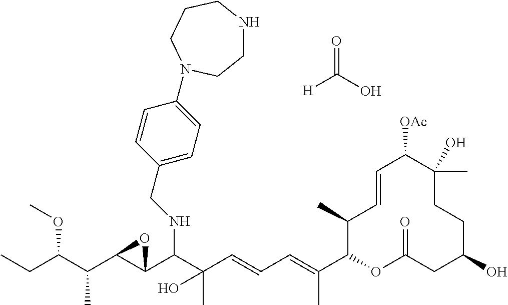 | 770.5 770.5 | 3.225 3.172 | 0-50- 100B2AC 110 min |
| MC-042a MC-042b MC-042c | 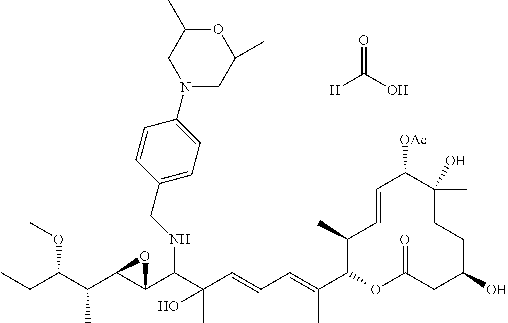 | 785.5 785.5 785.5 | 1.552-91% 1.416-9% 1.418 1.535 | 25- 100B2AC 110 min |
| MC-043a MC-043b MC-043c | 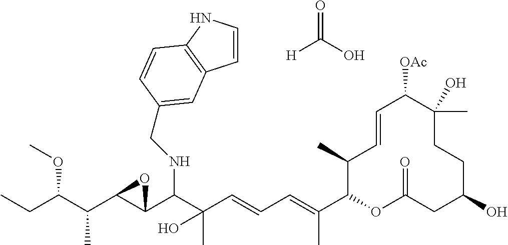 | 711.4 711.4 711.4 | 3.924 3.882 3.900 | 0-50- 100B2AC 110 min |
| MC-044a MC-044b | 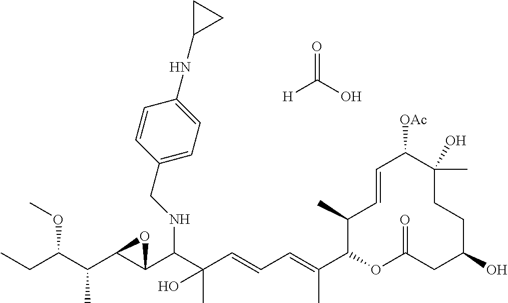 | 727.5 727.5 | 1.567 1.591 | 25- 100B2AC 110 min |
| MC-045 | 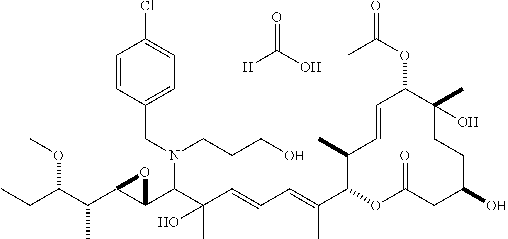 | 764.4 | 1.75 | 25- 100B2AC 110 min |
| MC-046a MC-046b | 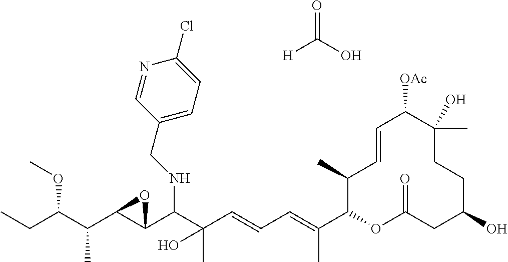 | | 3.506 3.623 3.549 | 0-50- 100B2AC 110 min |
| MC-047a MC-047b | 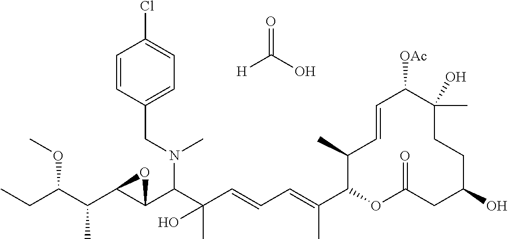 | | 3.989 4.031 | 0-50- 100B2AC 110 min |
| MC-048 | 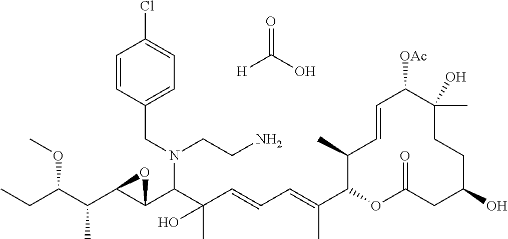 | 749.4 | 5.017 | 0-40- 100B2AC 110 min |
| MC-049 | 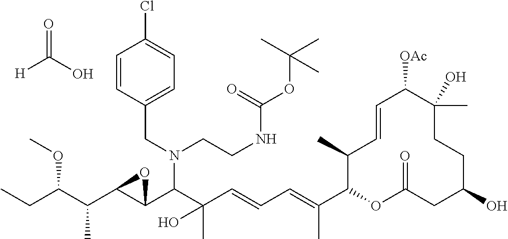 | 849.5 | 2.268 | 25- 100B2AC 110 min |
| MC-050a MC-050b | 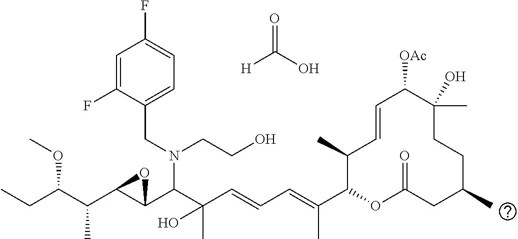 | 774.4 774.4 | 1.497 1.591 | 25- 100B2AC 110 min |
| MC-051 | 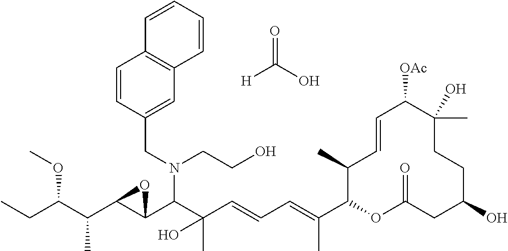 | 766.3 | 1.830 | 25- 100B2AC 110 min |
| MC-052 | 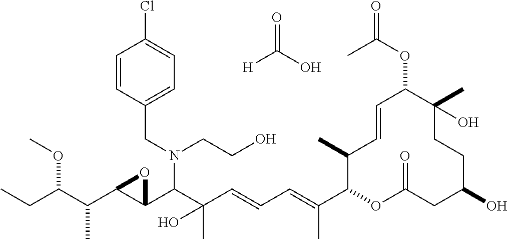 | 750.4 | 1.66 | 25- 100B2AC 110 min |
| MC-053 | 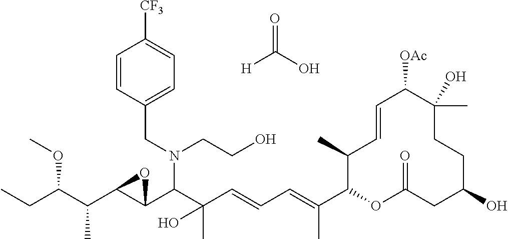 | 784.4 | 1.662 | 25- 100B2AC 110 min |
| MC-054a MC-054b | 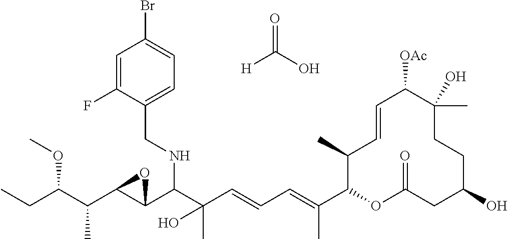 | 770.3 770.3 | 1.474-83.6% 1.366-16.4% 1.450 | 25- 100B2AC 110 min |
| MC-055a MC-055b | 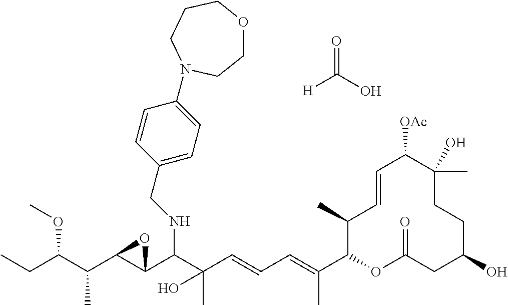 | 771.5 771.5 | 2.343 2.483 | 25- 100B2AC 110 min |
| MC-056a MC-056b MC-056c | 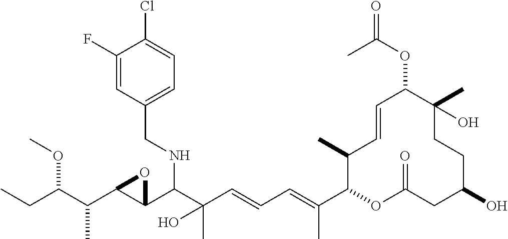 | | | |
| MC-057a MC-057b | 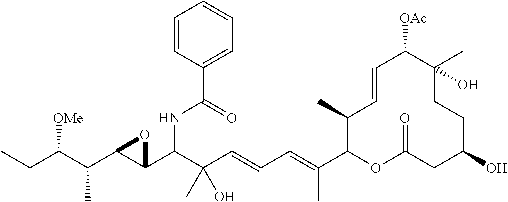 | 686.3800 686.3778 | 1.677 1.834 | 25-100% AcCN/H₂O 25-100% AcCN/H₂O |
| MC-058a MC-058b | 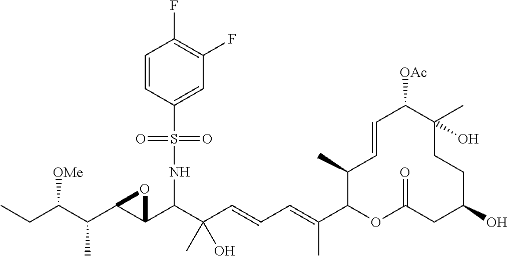 | 780.3236 780.3245 | 2.998 2.990 | 25-100% AcCN/H₂O 25-100% AcCN/H₂O |
| MC-059 | 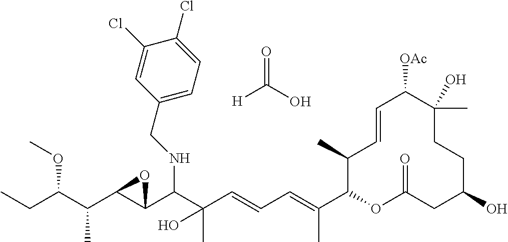 | 740.3 | 1.814 | 25- 100B2AC 110 min |

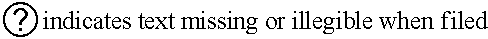

Biological Methods

Growth Inhibition of Various Human Cancer Cell Lines by MC Compounds

The cytotoxicity of each of the MC compounds as described herein was evaluated in the following cell types: A549 human alveolar epithelial cell, PC3 human prostate carcinoma cell, COLO205 human colon adenocarcinoma cell, HEPG2 human hepatoma, OVCAR3 human ovary adenocarcinoma cell, and HL60 human acute myelocytic leukemia (AML) cell.

A549, PC3, COLO205 and HEPG2 cells were cultured in DMEM (Invitrogen Corporation) with 10% fetal bovine serum, whereas OVCAR3 and HL60 were cultured in the same medium but with 20% fetal bovine serum. For cytotoxocoty measurements the cells were seeded in 96 or 384 well tissue culture treated microtitre plates overnight. Compounds were diluted in appropriate buffers and added to the cells the next day. Following a further 72 hour incubation, cell viability was determined using Cell Titer Glo (Promega Corporation) according to the manufacturers' instructions, and the luminescence measured in Luminoskan Ascent (Thermo Fisher Scientific Inc.).

Microsomal Stability Assays

The samples were prepared in duplicates in 96-well plate format as follows with final volume of 0.5 mL and test sample concentration of 5 μM. 50 μL test sample (50 mM), 25 μL human liver microsomes (20 mg/ml, BD Gentest, catalog no. 452161), 281 μL potassium phosphate buffer (100 mM, pH 7.4), 5 μL NaDPH regenerating system, solution B (BD Biosciences, catalog no. 451200), and 114 μL of water were pre-incubated for 5 min at 37° C. The reaction was initiated by the addition of 25 μL NaDPH regenerating system, solution A (BD Biosciences, catalog no. 451220). After an incubation period of 0, 15, 30, and 60 min at 37° C., 100 μL of the reaction mixture was transferred to an eppendorf tube, followed by addition of 100 μL chilled acetonitrile to quench the reaction. The sample was then centrifuged at 14,000 rpm for 5 min. The supernatant was submitted for LCMS analysis.

Stability Determination at PBS pH7.2

Around 0.1 mg of the dried sample was placed in a 2-mL vial, added with 1 mL of PBS pH7.2 and then mixed using a Vortex mixer. The mixture was filtered through a 4 mm Nylon filter with 0.2 μm pore size and the filtrate collected in an hplc vial. The vial was placed in an hplc autosampler maintained at 37° C. just before injection (time=0 h). The stability of the sample was calculated by determining the amount left in solution at specific time intervals. This study was carried out in three trials with the buffer solution as a blank.

Biological Data

Biological data were obtained using the A549 assay described above for the following compounds: MC-016a, MC-016b, MC-004a, MC-004b, MC-005, MC-006c, MC-006a, MC-059, MC-007b, MC-007c, MC-007d, MC-046a, MC-046b, MC-014a, MC-014b, MC-018a, MC-027a, MC-027b, MC-028a, MC-28b, MC-026a, MC-026b, MC-026c, MC-026d, MC-030a, MC-030b, MC-029, MC-031, MC-032, MC-056a, MC-056b, MC-056c, MC-033, MC-035, MC-037d, MC-055a, MC-055b, MC-038, MC-039, MC-041a, MC-041b, MC-041c, MC-042a, MC-042b, MC-042c, MC-043a, MC-043b, MC-043c, MC-044a, MC-044b, MC-012a, MC-012b, MC-001, MC-008c, MC-036, MC-003, MC-047a, MC-047b, MC-052, MC-045, MC-053, MC-051, MC-050a, MC-050b, MC-049, MC-048, MC-017b, MC-017a, MC-009b, MC-011, MC-013, MC-019b, MC-019c, MC-022, MC-034, MC-58b, MC-057a and MC-057b.

For the A549 assay, all of the compounds had IC50 values of less than 40 μM.

For the A549 assay, the following compounds had IC50 values of 1 μM or less: MC-016a, MC-016b, MC-004a, MC-004b, MC-005, MC-006c, MC-006a, MC-059, MC-007b, MC-007c, MC-046a, MC-046b, MC-014a, MC-014b, MC-018a, MC-027a, MC-027b, MC-028a, MC-28b, MC-026a, MC-026b, MC-026c, MC-026d, MC-030a, MC-030b, MC-029, MC-031, MC-032, MC-056a, MC-056b, MC-056c, MC-033, MC-037d, MC-055a, MC-055b, MC-039, MC-041a, MC-042a, MC-042b, MC-042c, MC-043a, MC-043b, MC-043c, MC-044a, MC-044b, MC-012a, MC-001, MC-047a, MC-047b, MC-052, MC-045, MC-053, MC-051, MC-050a, MC-050b, MC-017b, MC-017a, MC-034 and MC-58b.

For the A549 assay, the following compounds had IC50 values of 100 nM or less: MC-016a, MC-016b, MC-004a, MC-004b, MC-005, MC-006c, MC-006a, MC-059, MC-046a, MC-046b, MC-014a, MC-014b, MC-28b, MC-026a, MC-026b, MC-026c, MC-026d, MC-030a, MC-030b, MC-029, MC-031, MC-032, MC-056a, MC-056b, MC-056c, MC-033, MC-037d, MC-055a, MC-039, MC-042a, MC-043a, MC-047a, MC-047b and MC-052.

One compound, compound MC-005, had an IC50 value of 53 nM.

Biological data were obtained using the COLO205 assay described above for the following compounds: MC-016a, MC-016b, MC-005, MC-006c, MC-006a, MC-059, MC-007b and MC-007c.

For the COLO205 assay, all of the compounds had IC50 values of less than 1 μM.

For the COLO205 assay, the following compounds had IC50 values of 0.1 μM or less: MC-016a, MC-005, MC-006c, MC-059 and MC-007c.

One compound, compound MC-005, had an IC50 value of 34 nM.

Biological data were obtained using the HEPG2A assay described above for the following compounds: MC-016a, MC-016b, MC-005, MC-006c, MC-006a, MC-059, MC-007b and MC-007c.

For the HEPG2A assay, all of the compounds had IC50 values of less than 1 μM.

For the HEPG2A assay, the following compounds had IC50 values of 0.1 OA or less: MC-016a, MC-016b, MC-005 and MC-059.

One compound, compound MC-005, had an IC50 value of 57 nM.

Biological data were obtained using the HL60A assay described above for the following compounds: MC-016a, MC-016b, MC-005, MC-006c, MC-006a, MC-059, MC-007b and MC-007c.

For the HL60A assay, all of the compounds had IC50 values of less than 1 μM.

For the HL60A assay, the following compounds had IC50 values of 0.1 μM or less: MC-016a, MC-016b, MC-005, MC-006c, MC-006a, MC-007b and MC-007c.

One compound, compound MC-005, had an IC50 value of 48 nM.

Biological data were obtained using the OVCAR3 assay described above for the following compounds: MC-016a, MC-016b, MC-005, MC-006c, MC-006a, MC-059, MC-007b, MC-007c, MC-030a, MC-029, MC-031 and MC-032.

For the OVCAR3 assay, all of the compounds had IC50 values of less than 1 μM.

For the OVCAR3 assay, the following compounds had IC50 values of 0.1 μM or less: MC-016a, MC-016b, MC-005, MC-006c, MC-059, MC-030a, MC-029, MC-031 and MC-032.

One compound, compound MC-005, had an IC50 value of 61 nM.

Biological data were obtained using the PC3A assay described above for the following compounds: MC-016a, MC-016b, MC-005, MC-006c, MC-006a, MC-059, MC-007b, MC-007c, MC-030a, MC-029, MC-031 and MC-032.

For the PC3A assay, all of the compounds had IC50 values of less than 1 μM.

For the PC3A assay, the following compounds had IC50 values of 0.1 μM or less: MC-016a, MC-016b, MC-005, MC-006c, MC-006a, MC-059, MC-030a, MC-029, MC-031 and MC-032.

One compound, compound MC-005, had an IC50 value of 32 nM.

Stability data was obtained using the 5 μm microsomal stability assay described above for MC-034. For the 5 μm microsomal assay, MC-034 had a T_{1/2} value of 6.1 minutes.

Stability data were obtained using the 50 μm microsomal stability assay described above for the following compounds: MC-007d, MC-005, MC-006c, MC-014b, MC-016a, MC-029, MC-030a, MC-031, MC-032, MC-056a, MC-043a, MC-052, MC-047a, MC-046a, MC-037d, MC-017b and MC-009b.

For the 50 μm microsomal assay, all of the compounds tested had a T_{1/2} value of more than 10 minutes.

For the 50 μm microsomal assay, the following compounds had a value of more than 30 minutes: MC-006c, MC-014b, MC029, MC-030a, MC-031, MC-032, MC043a, MC-046a, MC-037d and MC-009b.

For the 50 μm microsomal assay, the following compounds had a T_{1/2} value of more than 60 minutes: MC-030a, MC-031, MC-032 and MC043a.

One compound, compound MC-005, had a T₁₁₂ value of 20 minutes.

Stability data were obtained using the PBS buffer stability assay described above for the following compounds: MC-034, MC-016, MC-029, MC-030, MC-031, MC-032, MC-056, MC-043, MC-052, MC-050, MC-047, MC-046 and MC-037.

For the PBS buffer stability assay, all of the compounds tested had a T_{1/2} value of more than 15 minutes.

For the PBS buffer stability assay, the following compounds had a T_{1/2} value of more than 30 minutes: MC-034, MC-030, MC-031, MC-043, MC-052, MC-050, MC-047, MC-046 and MC-037.

For the PBS buffer stability assay, the following compounds had a T₁₁₂ value of more than 100 minutes: MC-034, MC-052 and MC-050.

One compound, compound MC-034, had a T_{1/2} value of 164 hours.

The foregoing has described the principles, preferred embodiments, and modes of operation of the present invention. However, the invention should not be construed as limited to the particular embodiments discussed. Instead, the above-described embodiments should be regarded as illustrative rather than restrictive, and it should be appreciated that variations may be made in those embodiments by workers skilled in the art without departing from the scope of the present invention.

### REFERENCES

A number of patents and publications are cited above in order to more fully describe and disclose the invention and the state of the art to which the invention pertains. Full citations for these references are provided below. Each of these references is incorporated herein by reference in its entirety into the present disclosure, to the same extent as if each individual reference was specifically and individually indicated to be incorporated by reference.
WO07110704, “Macrolide Compounds As Therapeutic Agents”
WO07110705, “Macrolide Compounds As Therapeutic Agents”
EP1380579A, “Novel Physiologically Active Substances”
Abdel-Magid, A. F., et al, J. Org. Chem. 1996, 61, 3849-3862
Shafir, A., et al, JACS 2006, 128, 8742-8743

1. A compound selected from compounds of the following formula, and pharmaceutically acceptable salts, hydrates, and solvates thereof: 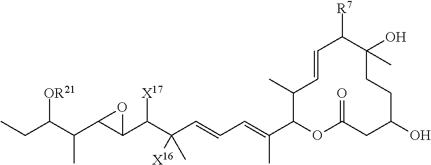
wherein:
—R⁷ is independently —OH, —OR^{7A}, or —O—C(O)R^{7A}
wherein:
—R^{7A} is independently —R^{7A1}, —R^{7A2} or —R^{7A3}
wherein:
—R^{7A1} is independently saturated or unsaturated aliphatic C₁₋₆alkyl, and is optionally substituted
and wherein:
—R^{7A2} is independently saturated or unsaturated alicyclic or aromatic C₃₋₂₀carbocyclyl, and is optionally substituted
and wherein:
—R^{7A3} is independently —NH₂, —NHR^{7NA}, —N(R^{7NA})₂, or —NR^{7NB}R^{7NC}
wherein:
each —R^{7NA} is independently —R^{7NA1} or —R^{7NA2}
wherein:
—R^{7NA1} is independently saturated or unsaturated aliphatic C₁₋₆alkyl, and is optionally substituted
and wherein:
—R^{7NA2} is independently saturated or unsaturated alicyclic or aromatic, carbo or hetero, C₃₋₂₀cyclyl, and is optionally substituted
and wherein:
—NR^{7NB}R^{7NC} is independently saturated or unsaturated alicyclic or aromatic C₃₋₂₀heterocyclyl, and is optionally substituted
and wherein:
—X¹⁶ is independently —OR^{16A} or —NR^{16A}R^{16B}
wherein:
R^{16A} is independently —H, —Z^{16A}, —Y^{16A}Z^{16A} or together with —R^{17A} is -L-
wherein:
—Y^{16A}— is independently saturated or unsaturated aliphatic C₁₋₄ alkylene, and is optionally substituted
and wherein:
-L- is independently saturated or unsaturated aliphatic C₁₋₄ alkylene, —S(O)(O)— or —C(O)—, and is optionally substituted
and wherein:
—Z^{16A} is independently —Z^{16A1} or —Z^{16A2}
wherein:
—Z^{16A1} is independently saturated or unsaturated aliphatic C₁₋₆alkyl, and is optionally substituted
and wherein:
—Z^{16A2} is independently saturated or unsaturated alicyclic or aromatic C₃₋₂₀cyclyl, and is optionally substituted
and wherein:
R^{16B} is independently —H, —Z^{16B} or —Y^{16B}Z^{16B}
wherein:
—Y^{16B}— is independently saturated or unsaturated aliphatic C₁₋₄ alkylene, and is optionally substituted
and wherein:
—Z^{16B} is independently —Z^{16B1} or —Z^{16B2}
wherein:
—Z^{16B1} is independently saturated or unsaturated aliphatic C₁₋₆alkyl, and is optionally substituted
and wherein:
—Z^{16B2} is independently saturated or unsaturated alicyclic or aromatic C₃₋₂₀cyclyl, and is optionally substituted
and wherein:
—X¹⁷ is independently —NR^{17A}R^{17B}
wherein:
—R^{17A} is independently —H, —Z^{17A}, —Y^{17A}Z^{17A} or together with —R^{16A} is -L-
wherein:
—Y^{17A}— is independently saturated or unsaturated aliphatic C₁₋₄ alkylene, —C(O)—, or —S(O)(O)—, and is optionally substituted
and wherein:
-L- is independently as defined above
and wherein:
—Z^{17A} is independently —Z^{17A1} or —Z^{17A2}
wherein:
—Z^{17A1} is independently saturated or unsaturated aliphatic C₁₋₆alkyl, and is optionally substituted
and wherein:
—Z^{17A2}, if present, is independently —Z^{17AH} or —Z^{17AC}
wherein:
—Z^{17AH} is saturated or unsaturated alicyclic or aromatic C₃₋₂₀heterocyclyl, and is optionally substituted
and wherein:
—Z^{17AC} is saturated or unsaturated alicyclic or aromatic C₃₋₂₀carbocyclyl, and is optionally substituted
and wherein:
—R^{17B} is independently —H, —Z^{17B} or —Y^{17B}Z^{17B},
wherein:
—Y^{17B}— is independently saturated or unsaturated aliphatic C₁₋₄ alkylene, —C(O)—, or —S(O)(O)—, and is optionally substituted
and wherein:
—Z^{17B} is independently —Z^{17B1}, —Z^{17B2} or —Z^{17B3}.
wherein:
—Z^{17B1} is independently saturated or unsaturated aliphatic C₁₋₆alkyl, and is optionally substituted
and wherein:
—Z^{17B2} is independently saturated or unsaturated alicyclic or aromatic C₃₋₂₀carbocyclyl, and is optionally substituted
and wherein:
—Z^{17B3} is independently saturated or unsaturated alicyclic or aromatic C₃₋₂₀heterocyclyl, and is optionally substituted
and wherein:
—R²¹ is independently —H or -Me.

2. A compound according to claim 1, wherein —X¹⁶ is independently —OH, —NZ^{16A}H, N—Y^{16A}Z^{16A2}H or together with —R^{17A} is -L-.
wherein:
—Y^{16A}—, if present, is independently saturated or unsaturated C₁₋₂alkylene, and is optionally substituted.
and wherein:
—Z^{16A2}, if present, is independently saturated or unsaturated alicyclic or aromatic C₅₋₇carbocyclyl, and is optionally substituted.
and wherein:
-L-, if present, is independendently —CH₂—CH₂—, —CH₂—CH₂—CH₂—, —CH(Ph)—CH(Ph)— or —C(O)—.

3. A compound according to claim 2, wherein —X¹⁶ is independently —OH, N—Y^{16A}Z^{16A2}H or together with —R^{17A} is -L-.

4. A compound according to claim 3, wherein —X¹⁶ is independently —OH.

5. A compound according to any one of claims 1 to 4, wherein R^{17B} is independently —H or —Z^{17B1}.

6. A compound according to any one of claims 1 to 5, wherein R^{17A} is independently —H, —Y^{17A}Z^{17A2} or together with —R^{16A} is -L-,
wherein:
—Z^{17A2}, if present, is independently —Z^{17AH} or —Z^{17AC}
wherein:
—Z^{17AH} is saturated or unsaturated alicyclic or aromatic C₅₋₇heterocyclyl, and is optionally substituted
and wherein:
—Z^{17AC} is saturated or unsaturated alicyclic or aromatic C₅₋₇carbocyclyl, and is optionally substituted.

7. A compound according to claim 6, wherein R^{17A} is independently —H, —Y^{17A}Z^{17A2} or together with —R^{16A} is -L-,
wherein:
—Y^{17A}—, if present, is independently —CH₂—, —C(CH₃)H—, —CH₂—CH₂—, —CH₂—CH₂—CH₂—, —C(O)— or —S(O)(O)—.
and wherein:
—Z^{17A2} if present, is independently —Z^{17AH} or —Z^{17AC}
wherein:
—Z^{17AH} is saturated or unsaturated alicyclic or aromatic C₆heterocyclyl, and is optionally substituted
and wherein:
—Z^{17AC} is saturated or unsaturated alicyclic or aromatic C₆carbocyclyl, and is optionally substituted.
and wherein:
-L-, if present, is independendently —CH₂—CH₂—, —CH₂—CH₂—CH₂—, —CH(Ph)—CH(Ph)— or —C(O)—.

8. A compound according to any one of claims 1 to 7, wherein —R⁷ is —O—C(O)R^{7A1}, wherein —R^{7A1} is independently saturated or unsaturated aliphatic C₁₋₃alkyl, and is optionally substituted.

9. A compound according to any one of claims 1 to 8, wherein —R²¹ is independently -Me.

10. A compound according to any one of claims 1 to 9, wherein:
each of —R^{7A1}, if present, —R^{7NA1}, if present, —Z^{16A1}, if present, —Z^{16B1}, if present, —Z^{17A1}, if present, and —Z^{17B1}, if present, is optionally substituted with one or more substituents, —R^{S1}, wherein each —R^{S1} is independently selected from:
—R^{JA1},
—F, —Cl, —Br, —I—
—CF₃,
—OH, -L^{JA}-OH, —O-L^{JA}-OH,
—OR^{JA1}, -L^{JA}-OR^{JA1},
—CN,
—NH₂, —NHR^{JA1}, —NR^{JA1}₂, and
—NHC(═O)OR^{JA1}, —NR^{JA1}C(═O)OR^{JA1};
each of —R^{7A2}, if present, —R^{7NA2}, if present, —Z^{16A2}, if present, and —Z^{16B2}, if present, is optionally substituted with one or more substituents, —R^{S2}, wherein each —R^{S2} is independently selected from:
—R^{JA1},
—F, —Cl, —Br, —I—
—CF₃,
—OH, -L^{JA}-OH,
—OR^{JA1}, -L^{JA}-OR^{JA1},
—CN,
—NH₂, —NHR^{JA1}, —NR^{JA1}₂, —NR^{JA2}R^{JA3},
-L^{JA}-NH₂, -L^{JA}-NHR^{JA1}, -L^{JA}-NR^{JA1}₂,
—C(═O)OH,
—C(═O)R^{JA1},
—C(═O)NH₂, —C(═O)NHR^{JA1}, and —C(═O)NR^{JA1}₂; and
two adjacent groups —R^{S2}, if present, together form —O—CH₂—O— or —O—CH₂CH₂—O—;
each of —NR^{7NB}R^{7NC}, if present, —Z^{16B3}, if present, and —Z^{17AH}, if present, is optionally substituted with one or more substituents, —R^{S3}, wherein each —R^{S3} is independently selected from:
—R^{JA1},
—F, —Cl, —Br, —I—
—CF₃,
—OH, -L^{JA}-OH,
—OR^{JA1}, -L^{JA}-OR^{JA1},
—CN,
—NH₂, —NHR^{JA1}, —NR^{JA1}₂, —NR^{JA2}R^{JA3},
-L^{JA}-NH₂, -L^{JA}-NHR^{JA1}, -L^{JA}-NR^{JA1}₂,
—C(═O)OH,
—C(═O)R^{JA1},
—C(═O)NH₂, —C(═O)NHR^{JA1}, and —C(═O)NR^{JA1}₂; and
two adjacent groups —R^{S3}, if present, together form —O—CH₂—O— or —O—CH₂CH₂—O—;
each of —Z^{17AC}, if present, —Z^{17B2}, if present, and —Z^{17B3}, if present, is optionally substituted with one or more substituents, —R^{S4}, wherein each —R^{S4} is independently selected from:
—R^{JA1},
—F, —Cl, —Br, —I—
—CF₃,
—OH, -L^{JA}-OH,
—OR^{JA1}, -L^{JA}-OR^{JA1},
—CN,
—NH₂, —NHR^{JA1}, —NR^{JA1}₂, —NR^{JA2}R^{JA3},
-L^{JA}-NH₂, -L^{JA}-NHR^{JA1}, -L^{JA}-NR^{JA1}₂,
—C(═O)OH,
—C(═O)R^{JA1},
—C(═O)NH₂, —C(═O)NHR^{JA1}, and —C(═O)NR^{JA1}₂; and
two adjacent groups —R^{S4}, if present, together form —O—CH₂—O— or —O—CH₂CH₂—O—;
each of —Y^{16A}—, if present, —Y^{16B}—, if present, —Y^{17A}—, if present, —Y^{17B}—, if present, and -L-, if present, is optionally substituted with one or more substituents, —R^{S5}, wherein each —R^{S5} is independently selected from:
—R^{JA1},
—F, —Cl, —Br, —I—
—CF₃, —OCF₃,
—OH, -L^{JA}-OH,
—OR^{JA1},
—CN, and
—NH₂, —NHR^{JA1}, —NR^{JA1}₂;
wherein:
each -L^{JA}-, if present, is independently saturated aliphatic C₁₋₅alkylene;
each —NR^{JA2}R^{JA3}, if present, is independently C₃₋₁₀heterocyclyl, for example independently piperidino, piperazino, morpholino, oxazepino (e.g. homomorpholino) or diazepino (e.g. homopiperazino), and is optionally substituted, for example, with one or more groups selected from —R^{JJ}, —CF₃, —F, —OH, —OR^{JJ}, —NH₂, —NHR^{JJ}, —NR^{JJ}₂, and ═O;
wherein each —R^{JJ} is independently saturated aliphatic C₁₋₄ alkyl;
each —R^{JA1}, if present, is independently: —R^{JB1}, —R^{JB2}, —R^{JB3}, —R^{JB4}, —R^{JB5}, —R^{JB6}, —R^{JB7}, —R^{JB8}, -L^{JB}-R^{JB4}, -L^{JB}-R^{JB5}, -L^{JB}-R^{JB6}, -L^{JB}-R^{JB7}, or -L^{JB}- R^{JB8};
wherein:
each —R^{JB1} is independently saturated aliphatic C₁₋₆alkyl;
each —R^{JB2} is independently aliphatic C₂₋₆alkenyl;
each —R^{JB3} is independently aliphatic C₂₋₆alkynyl;
each —R^{JB4} is independently saturated C₃₋₆cycloalkyl;
each —R^{JB5} is independently C₃₋₆cycloalkenyl;
each —R^{JB6} is independently alicyclic C₄₋₇heterocyclyl;
each —R^{JB7} is independently C₆₋₁₀carboaryl;
each —R^{JB8} is independently C₅₋₁₀heteroaryl;
and wherein:
each -L^{JB}- is independently saturated aliphatic C₁₋₃alkylene;
wherein:
each —R^{JB4}, —R^{JB5}, —R^{JB6}, —R^{JB7}, and —R^{JB8} is optionally substituted, for example, with one or more substituents —R^{JC1} and/or one or more substituents —R^{JC2},
and wherein:
each —R^{JB1}, —R^{JB2}, —R^{JB3}, and -L^{JB}- is optionally substituted, for example, with one or more substituents —R^{JC2}, and
wherein:
each —R^{JC1} is independently saturated aliphatic C₁₋₄ alkyl, phenyl, or benzyl;
each —R^{JC2} is independently:
—F, —Cl, —Br, —I,
—CF₃, —OCF₃, —SCF₃,
—OH, -L^{JD}-OH, —O-L^{JD}-OH,
—OR^{JD1}, -L^{JD}-OR^{JD1}, —O-L^{JD}-OR^{JD1},
—SH, —SR^{JD1},
—CN,
—NO₂,
—NH₂, —NHR^{JD1}, —NR^{JD1}₂,
-L^{JD}-NH₂, -L^{JD}-NHR^{JD1}, -L^{JD}-NR^{JD1}₂,
—C(═O)OH, —C(═O)OR^{JD1},
—C(═O)NH₂, —C(═O)NHR^{JD1}, or —C(═O)NR^{JD1}₂;
wherein:
each —R^{JD1} is independently saturated aliphatic C₁₋₄alkyl, phenyl, or benzyl; and
each -L^{JD}- is independently saturated aliphatic C₁₋₅alkylene.

11. A compound according to any one of claims 1 to 10, wherein —R^{17A} is independently selected from the group consisting of:

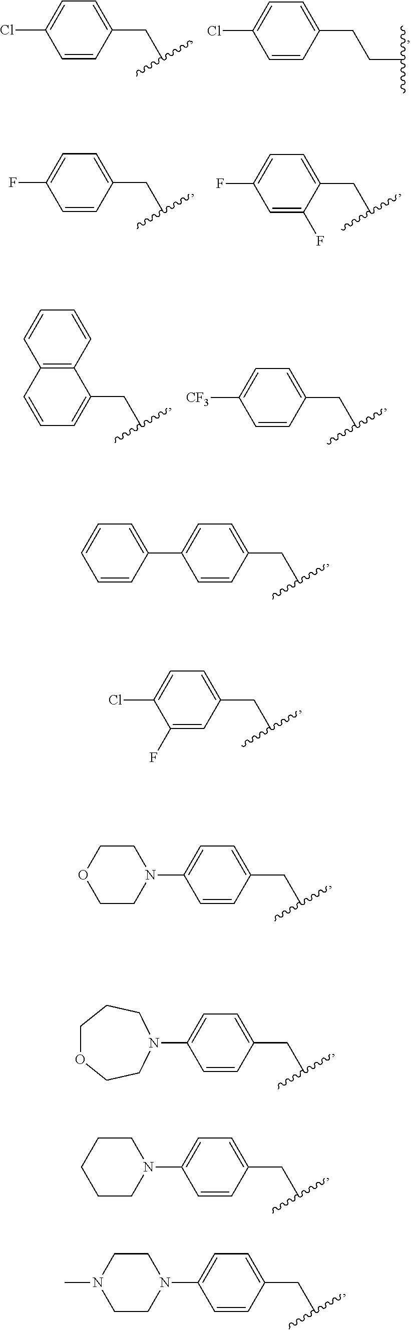

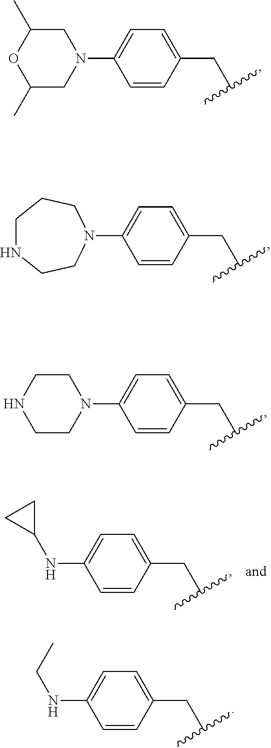

12. A compound according to claim 1, selected from the following compounds, and pharmaceutically acceptable salts, hydrates, and solvates thereof: Compound Nos. MC-001 to MC-059.

13. A pharmaceutical composition comprising a compound according to any one of claims 1 to 12, and a pharmaceutically acceptable carrier, diluent, or excipient.

14. A compound according to any one of claims 1 to 12, for use in a method of treatment of cancer.

15. A method of treatment of cancer comprising administering to a subject in need of treatment a therapeutically effective amount of a compound according to any one of claims 1 to 12.
